(19) United States
(12) Patent Application Publication
(10) Pub. No.: US 2026/0125634 A1
(43) Pub. Date: May 7, 2026

(54) Genetically Engineered Yeast Producing 3-Hydroxypropionic Acid at Low pH

(71) Applicant: ARCHER-DANIELS-MIDLAND COMPANY, Decatur, IL (US)

(72) Inventors: Owen Ryan, Decatur, IL (US); Alexander Chou, Decatur, IL (US); Jesús Seco Moral, Decatur, IL (US); Marina Canellas, Decatur, IL (US); Jamie Ryding, Decatur, IL (US)

(21) Appl. No.: 19/108,144

(22) PCT Filed: Aug. 29, 2023
(86) PCT No.: PCT/US2023/073109

§ 371 Date: Feb. 28, 2025

Related U.S. Application Data
(60) Provisional application No. 63/401,986, filed on Aug. 29, 2022.

(51) Int. Cl. C12N 1/18; C12N 9/00; C12N 9/04; C12N 9/88; C12P 7/42; C12Q 1/26; C12Q 1/527; C12R 1/645

(52) U.S. Cl. CPC C12N 1/18; C12N 9/0006; C12N 9/88; C12N 9/93; C12P 7/42; C12Q 1/26; C12Q 1/527; C12Y 101/0106; C12Y 401/01003; C12Y 401/01031; C12Y 401/01032; C12Y 604/01001; C12R 2001/645

(57) ABSTRACT

Described herein is a novel enzymatic pathway and acid tolerant Schizosaccharomyces pombe strain exhibiting the same, for producing 3-hydroxypropionic acid by fermentation wherein the S. pombe strain includes nucleotide sequences that encode, and which are operably linked to promoters to express, non-native enzymes that exhibit an oxaloacetate decarboxylase (ODC) activity that converts oxaloacetate to 3-oxopropionate and a 3-oxopropionate reductase (OPR) activity that converts the 3-opropionate to 3-hydroxypropianate. ODC and OPR enzymes are not known to exist in nature, nor is an enzymatic path for making 3-hydroxypropionic acid but particular enzymes exhibiting the requisite activities are herein identified by sequence. A further enhancement is to also overexpress an enzyme that has at least one of a pyruvate carboxylase activity, a phosphoenolpyruvate carboxy kinase activity, and a phosphoenolpyruvate carboxylase activity, which increases the level of oxaloacetate in the cell leading to greater 3-HP production.

### TECHNICAL FIELD

The invention relates to the production of 3-hydroxypropionic acid (3-HP) by fermentation of a microorganism genetically engineered to express an enzyme that exhibits a novel oxaloacetate decarboxylase (ODC) activity that converts oxaloacetate to 3-oxopropionate and a novel 3-oxopropionate reductase (OPR) activity that converts 3-oxopropionate to 3-HP. More particularly the microorganism is an acid tolerant yeast, and in exemplary embodiments the microorganism is a strain of Schizosaccharomyces pombe.

### SEQUENCE LISTING

The instant application contains a Sequence Listing which has been submitted electronically in XML format and is hereby incorporated by reference in its entirety. Said XML copy, created on Aug. 29, 2023, is named “Sequence Listing BIO0071US00_ST26” and is 193 KB in size.

### BACKGROUND OF THE INVENTION

3-hydroxypropionic acid (3-HP) is a biological molecule that is a promising starting material for the production of acrylic acid, acrolein and other three carbon monomers that can be used as renewable resources to produce polymers from biological material. There are several routes to produce 3-HP from glucose via fermentation described in the academic and patent literature. In one approach, glycerol is dehydrated and oxidized using an oxygen-sensitive and vitamin B12 dependent enzyme. In an alternative, 3-HP may be produced by reduction of the metabolite malonyl-CoA. Malonyl-CoA is produced from the ATP-dependent carboxylation of acetyl-CoA, which is highly compartmentalized and regulated in yeast. 3-HP has also been produced via aspartate and beta-alanine as intermediates, requiring superfluous amination and de-amination steps. S. cerevisiae and E. coli, have been engineered for 3-HP production according to the above routes but in each case the fermentation process using these organisms requires that the pH of the media be maintained in a suitable range over the course of fermentation. This leads to increased costs to produce and isolate the 3-HP product.

U.S. Pat. No. 9,365,875 discloses yeast cells engineered to produce 3-HP with enhanced production by the expression of a NADP-dependent glyceraldehyde-3-phosphate dehydrogenase.

U.S. Pat. No. 9,845,484 discloses yeast cells engineered to produce 3-HP having a 3-HP pathway and further expressing an aspartate 1-decarboxylase of the class Insecta, Bivalvia, Branchioporia, Gastropoda, or Leptocardii. This disclosure relies on use of an aspartate 1-decarboxylase.

U.S. Pat. No. 8,883,464 discloses recombinant microorganisms producing 3-HP from glucose via a pathway consisting of glycolysis, conversion of pyruvate to acetyl-CoA, carboxylation of acetyl-CoA to malonyl-CoA, reduction of malonyl-CoA to 3-oxopropionic acid, and reduction of 3-oxopropionic acid to 3-HP. Sequences of some enzymes catalyzing the reaction steps of the pathway are disclosed. This disclosure does not disclose a step of decarboxylation of oxaloacetate.

U.S. Pat. No. 9,447,438 discloses microorganisms producing 3-HP by expressing acetyl-CoA carboxylase, which converts acetyl-CoA to malonyl-CoA. Further, malonyl-CoA is reduced by malonyl-CoA reductase to malonate semialdehyde (3-oxopropionic acid) and malonate semialdehyde is converted to 3-HP by 3-HP dehydrogenase (ydfG, mmsB, NDSD, rutE, nemA or homolog). This disclosure does not disclose a step of decarboxylation of oxaloacetate.

T. Tong, et al., A biosynthesis pathway for 3-hydroxypropionic acid production in genetically engineered Saccharomyces cerevisiae, Green Chem., 2021, DOI: 10.1039/DOGC04431H reports the engineering of S. cerevisiae for the production of 3-HP from glucose via a pathway consisting of glycolysis, pyruvate carboxylation to oxaloacetate, oxaloacetate decarboxylation to 3-oxopropionic acid, and 3-oxopropionic acid reduction to 3-hydroxypropionic acid. The steps following glycolysis are reportedly catalyzed by pyruvate carboxylase (Pyc), benzoylformate decarboxylase (MdlC from Pseudomonas putida E23) or branched-chain alpha-ketoacid decarboxylase (KdcA from Lactococcus lactis), and 3-hydroxyisobutyrate dehydrogenase (MmsB from Pseudomonas putida E23), respectively. The strain of S. cerevisiae is further engineered to divert carbon flux to pyruvate (instead of glycerol or ethanol) and further optimizations are made to improve 3-HP production by control of intracellular ATP. A final titer of 18.3 g/L 3-HP is reported. S. cerevisiae is not an acid tolerant yeast so production of 3-HP in these organisms will be limited by its pH tolerance.

Akiko Suyama, et al, Production of 3-hydroxypropionic acid via the malonyl-CoA pathway using recombinant fission yeast strains, Journal of Bioscience and Bioengineering vol. 124 No. 4, 392-399 (2017) reports the engineering of S. pombe for the production of 3-HP from glucose via a pathway consisting of glycolysis, conversion of pyruvate to acetyl-CoA, carboxylation of acetyl-CoA to malonyl-CoA, reduction of malonyl-CoA to 3-oxopropionic acid, and reduction of 3-oxopropionic acid to 3-HP. The authors report the heterologous expression of the enzymes acetyl-CoA carboxylase (Cut6p from S. pombe) and malonyl-CoA reductase (MCR from C. aurantiacus) to catalyze the final three steps of the pathway. The authors also report that acetate supplementation improves 3-HP production, reaching a titer of 7.6 g/L. The enzymatic pathway reported therein does relies on carboxylation of aceytyle-CoA and does not involve decarboxylation of oxaloacetate.

Similarly, Seiya Takayama et al, Enhancing 3-hydroxypropionic acid production in combination with sugar supply engineering by cell surface-display and metabolic engineering of Schizosaccharomyces pombe, Microb Cell Fact (2018) 17:17; https://doi.org/10.1186/s12934-018-1025-5 reports the engineering of S. pombe for the production of 3-HP from glucose via the same pathway as above, consisting of glycolysis, conversion of pyruvate to acetyl-CoA, carboxylation of acetyl-CoA to malonyl-CoA, reduction of malonyl-CoA to 3-oxopropionic acid, and reduction of 3-oxopropionic acid to 3-HP. The authors report the heterologous expression of the enzymes acetyl-CoA carboxylase (Cut6p from S. pombe) and malonyl-CoA reductase (MCR from C. aurantiacus) to catalyze the final three steps of the pathway. The authors further describe methods to provide for additional intracellular acetyl-CoA and the use of cellobiose as the substrate. This is a first disclosure of a pathway that proceeds through malonyl CoA rather than oxaloacetate.

U.S. Pat. No. 8,048,624 discloses recombinant bacteria producing 3-HP by having identified enzymes with oxaloacetate alpha-decarboxylase (ODC) activity. The disclosure teaches the genetic engineering of a bacterium (namely E. coli) expressing ODC to convert oxaloacetate to 3-oxopropionate, and a dehydrogenase to convert 3-oxopropionate to 3-HP. Optionally, a pyruvate carboxykinase is used to improve 3-HP production via enhanced oxaloacetate production. The present disclosure describes the use of the enzymes pek (pyruvate carboxykinase), mmsA, mmsB (malonate semialdehyde dehydrogenases), oad-2 (alpha-oxo-decarboxylase) or homologues, as well as alpha-ketoglutarate decarboxylase (kgd from M. tuberculosis). The disclosure further teaches how kgd evolved into the oad-2 sequence by evolution. This disclosure relies on the bacterium E. coli as the production strain, which is not acid tolerant.

U.S. Pat. No. 8,809,027 discloses recombinant microorganisms producing 3-HP by having identified enzymes with improved oxaloacetate alpha-decarboxylase (ODC) activity. The disclosure names pyruvate decarboxylase from Zymomonas mobilis (pdc), 2-oxoglutarate decarboxylase from Leuconostoc mesenteroides (odc), and alpha-ketoglutarate decarboxylase from Mycobacterium tuberculosis (kgd). The invention discloses mutants of ODC from L. mesenteroides having up to 2.8 fold improvement in the conversion of oxaloacetate to 3-oxopropionate. Further described is the use of pyruvate carboxylase to supply oxaloacetate using pyc from Corynebacterium glutamicum or Rhizobium etli and the use of dehydrogenase to convert 3-oxopropionate to 3-HP (mmsB from Pseudomonas aeruginosa or mutated ydfG from E. coli). The disclosure also teaches the use of carbonic anhydrase to provide bicarbonate and the use of transhydrogenase to increase the pool of NADPH. The particular sequences for the ODC disclosed herein are not efficient for the decarboxylation of oxaloacetate and does not suggest use of an acid tolerant yeast.

U.S. Pat. No. 909,091 discloses genetically modified yeast cells having different routes to produce 3-HP. In the most relevant embodiment, glucose is converted to PEP or pyruvate by glycolysis. Pyruvate or PEP is carboxylated to oxaloacetate. Oxaloacetate is decarboxylated to 3-oxopropionate. 3-Oxopropionate is reduced to 3-HP. PEP or pyruvate is converted to oxaloacetate by phosphoenolpyruvate carboxylase or pyruvate carboxylase, respectively. Oxaloacetate is decarboxylated by a 2-keto acid decarboxylase, alpha-ketoglutarate decarboxylase, branched-chain alpha-keto acid decarboxylase, indolepyruvate decarboxylase, or benzoylformate decarboxylase. 3-oxopropionic acid is reduced by 3-HP dehydrogenase, 3-hydroxyisobutyrate dehydrogenase, or 4-hydroxyisobutyrate dehydrogenase. In some embodiments, the PYC is derived from I. orientalis, R. sphaeroides, R. etli, P. fluorescens, C. glutamicum, or S. meliloti and homologs. In some embodiments, the phosphoenolpyruvate carboxylase is derived from E. coli, M. thermoautotrophicum, or C. perfringens and homologs. In some embodiments, the 3-HP dehydrogenase is a homolog of I. orientalis YMR226C or S. cerevisiae YMR226C or E. coli ydfG. In some embodiments, the 3-hydroxyisobutyrate dehydrogenase is a homolog of A. faecalis M3A, P. putida mmsB, or P. aeruginosa mmsB. In some embodiments, 4-hydroxyisobutyrate dehydrogenase is a homolog of R. eutropha 4hbd or C. kluyveri hbd. Alpha-ketoglutarate decarboxylase is, in some embodiments, derived from a homolog of M. tuberculosis KGD, B. japnocum KGD, or M. loti KGD. A branched-chain alpha-keto acid decarboxylase is, in some embodiments, derived from a homolog of L. lactis kdcA. A benzoylformate decarboxylase is derived from P. putida mdlC. P. aeruginosa mdlC, P. stutzeri dpgB, or P. fluorescens ilvB-1 or homolog. The enzymes used for the decarboxylation of oxaloacetate and reduction of 3-oxopropionic acid are not efficient.

US2020/009562 discloses the production of 3-HP by a recombinant fungal host cell expressing an oxaloacetate decarboxylase and 3-hydroxypropionate dehydrogenase catalyzing the conversion of oxaloacetate to 3-oxopropionate and 3-oxopropionate to 3-HP, respectively. Oxaloacetate decarboxylases are selected from one of several sequences (Table 2 or Table 4). 3-hydroxypropionate dehydrogenase is selected from one of several sequences (SEQ ID NOS: 122-134). The enzyme for osxacolacetate decarboxylation and 3-oxopropionate reduction are not efficient in their respective activities.

There remains a need in the art for alternative enzymes for production of 3-HP production in appropriate acid tolerant microorganisms to enable economically viable fermentation processes that are readily scalable for commercial production. In particular, there is a need to discover better enzymes that exhibit oxaloacetate decarboxylase and 3-oxopropionate reductase activities that are operable in an acid tolerant yease such as Schizosaccharomyces pombe.

### SUMMARY OF THE INVENTION

The invention relates to genetically microorganisms that ferment dextrose to produce 3-hydroxypropionic acid (3-HP). The approach described herein involves a simplified biochemical pathway that is not known to exist in nature, which results in the conversion of the metabolite oxaloacetate to 3-HP in two enzyme catalyzed reaction steps. The enzymes with the desired function were identified based on computational prediction of the enzyme function and have not been described elsewhere. Optionally, the wild type enzymes are mutated to have improved activity for the desired reaction or reduced activity on undesired reactions.

The enzymes of the pathway are expressed in an exemplary acid tolerant yeast strain Schizosaccharomyces pombe, capable of growth and organic acid production at low pH. This is advantageous from an economic standpoint as it reduces the need for base addition to control pH during the fermentation and for acidification of the media after fermentation to isolate the 3-HP product.

With more particularity, in one aspect described herein is an acid tolerant yeast strain comprising nucleotide sequences that include a promoter sequence operable in the microorganism operably linked to coding sequences that encode non-native enzymes that exhibit a) an oxaloacetate decarboxylase (ODC) activity that converts oxaloacetate to 3-oxopropionate; and b) a 3-oxopropionate reductase (OPR) activity that converts the 3-opropionate to 3-hydroxypropianate wherein the enzyme that exhibits an OPR activity has a protein sequence selected from the group consisting of SEQ ID NOS: 1-58 or functional derivatives thereof, preferably SEQ ID NO: 26 or functional derivative thereof.

In particular embodiments of OPR sequences, the enzyme that exhibits an OPR activity has a protein sequence selected from the group consisting of SEQ ID NOS: 1-20 or functional derivative thereof. In still more particular embodiments, the enzyme that exhibits an OPR activity has a protein sequence selected from the group consisting of SEQ ID NOS: 1-5 or functional derivative thereof. In yet more particular embodiments, the enzyme that exhibits an OPR activity has a protein sequence selected from SEQ ID NOS: 1 or 3 or functional derivative thereof.

In certain particular embodiments of ODC sequences, the enzyme that exhibits an ODC activity has a protein sequence selected from the group consisting of SEQ ID NOS: 59-130 or functional derivatives thereof. In more particular embodiments the enzyme that exhibits an ODC activity has a protein sequence selected from the group consisting of SEQ ID NOS: 59-65 or functional derivatives thereof. In one most desirable embodiment, the enzyme that exhibits an ODC activity has a protein sequence according to SEQ ID NOS: 59 and 60 or functional derivatives thereof.

In further embodiments any of the foregoing acid tolerant yeast strains may further include a nucleotide sequence that encodes an enzyme that exhibits at least one of a pyruvate carboxylase activity, a phosphoenolpyruvate carboxy kinase activity, and a phosphoenolpyruvate carboxylase activity operably linked to a non-native promoter that expresses the enzyme in the acid tolerant yeast strain. In certain particular embodiments, the enzyme that exhibits at least one of a pyruvate carboxylase activity, a phosphoenolpyruvate carboxy kinase activity and a phosphoenolpyruvate carboxylase is at least one of a pyruvate carboxylase according to SEQ ID NO: 131 or a phosphoenolpyruvate carboxylase according to SEQ ID NO: 132, or functional derivative thereof.

In the most desirable embodiments of any of the above defined by OPR sequences, the acid tolerant yeast is most preferably a strain of Schizosaccharomyces pombe.

In another aspect, described herein are acid tolerant yeast strain that include a nucleotide sequences that include a promoter sequence operable in the microorganism operably linked to coding sequences that encode non-native enzymes that exhibit a) an oxaloacetate decarboxylase (ODC) activity that converts oxaloacetate to 3-oxopropionate, wherein the ODC activity is provided by an enzyme having a sequence selected from the group consisting of SEQ ID NOS: 59-130 or functional derivative thereof; and b) a 3-oxopropionate reductase (OPR) activity that converts the 3-opropionate to 3-hydroxypropianate.

In particular embodiments, the enzyme that exhibits an OPR activity has a protein sequence selected from the group consisting of SEQ ID NOS: 1-58 or functional derivative thereof, in more particular embodiments, the enzyme that exhibits an OPR activity has a protein sequence selected from the group consisting of SEQ ID NOS: 1-20 or functional derivative thereof. In still more particular embodiments, the enzyme that exhibits an OPR activity has a protein sequence selected from the group consisting of SEQ ID NOS: 1-4 or functional derivative thereof.

In certain particular embodiments the enzyme that exhibits an ODC activity has a protein sequence selected from the group consisting of SEQ ID NOS: 59-65 or functional derivative thereof. In most particular embodiments the enzyme that exhibits an ODC activity has a protein sequence according to SEQ ID NOS: 59 or functional derivative thereof.

In still further embodiments of the above, the acid tolerant yeast strain further includes a nucleotide sequence that encodes an enzyme that exhibits at least one of a pyruvate carboxylase activity, a phosphoenolpyruvate carboxy kinase activity, and a phosphoenolpyruvate carboxylase activity operably linked to a non-native promoter that expresses the enzyme in the acid tolerant yeast strain. In particular embodiments the enzyme that exhibits at least one of a pyruvate carboxylase activity, a phosphoenolpyruvate carboxy kinase activity and a phosphoenolpyruvate carboxylase is at least one of a pyruvate carboxylase according to SEQ ID NO: 131 or a according phosphoenolpyruvate carboxylase according to SEQ ID NO: 132, or functional derivative thereof.

In any of the forgoing embodiments defined by ODC sequence, the acid tolerant yeast is most preferably a strain of Schizosaccharomyces pombe.

In a totally different aspect, also provided herein is a method of determining whether a candidate enzyme increases oxaloacetate production in a microorganism comprising, expressing the candidate enzyme in the microorganism while simultaneously expressing an exogenous malate dehydrogenase gene in the microorganism and measuring the production of malic acid in the microorganism wherein the production of malic acid is determinative of whether the candidate enzyme increases oxaloacetate production in the microorganism.

### BRIEF DESCRIPTION OF THE DRAWINGS

FIG. 1 shows the enzymatic pathway of the present invention for production of 3-hydoxyproprionate from glucose by fermentation of a microorganism.

FIG. 2 shows exemplary purifications of 3-oxopropionate reductase (OPR) candidate enzymes analyzed by SDS-PAGE and Coomassie staining.

FIG. 3 shows absorption data from exemplary assays of OPR candidates on 3-oxopropionate with spectrophotometric monitoring of NADH consumption at 340 nm.

FIG. 4 shows the relative activities of candidate OPR variants (SEW ID NOS: 1-58 in descending order of activity) on the desired substrate: 3-oxopropionate (3OP), and undesired substrates: oxaloacetate (OAA), acetaldehyde (AALD), and pyruvate (PYR).

FIG. 5 shows exemplary 1H NMR detection of 3-HP formation by reduction of 3-oxopropionate with NADH as an electron donor using one candidate OPR enzyme (GAAR_ECOLI) of the present invention.

FIG. 6 shows exemplary assays of the activity of two candidate oxaloacetate decarboxylase (ODC) enzymes of the present invention (A0A3E2BIQ0_PRORE which is SEQ ID NO: 60 and A0A2K4C4J1_9STAP which is SEQ ID NO: 59) using oxaloacetate coupled with the OPR from GAAR_ECOLI with spectrophotometric monitoring of NADH consumption at 340 nm from the reductase reaction.

FIG. 7 shows the relative activities of candidate ODC variants (SEQ ID NOS: 59-130 in descending order of activity) on oxaloacetate (OAA) and pyruvate (PYR).

FIG. 8 shows exemplary 1H NMR detection of 3-HP formation from oxaloacetate to 3-HP in reactions containing both ODC and OPR enzymes of the present invention.

FIG. 9 shows a Western blot demonstrating expression of a histidine tagged OPR enzyme of the present invention in S. pombe.

FIG. 10 shows a Western blot demonstrating expression of a histidine tagged ODC enzyme of the present invention in S. pombe.

FIG. 11 shows an enzymatic pathway of the present invention for production of 3-hydoxyproprionate from glucose by fermentation of a microorganism coupled with a malate dehydrogenase enzyme useful for screening for oxaloacetate producing enzymes via malate production.

FIG. 12 is a chart showing increased production of oxaloacetate by S. pombe strains engineered to express an exogenous pyruvate carboxylase (PYC) or phosphoenolpyruvate carboxylase (PEPC) gene, where increased production of oxaloacetate is indirectly determined by measurement of increased production of malic acetate in the strains also engineered to overexpress malate dehydrogenase (MDH).

FIG. 13 shows Table 2 that lists useful candidate OPR enzymes of the present invention by SEQ ID NOS: 1-58.

FIG. 14 shows Table 3 that lists useful candidate OPR enzymes of the present invention by SEQ ID NOS: 59-130.

FIG. 15 shows Table 4 that lists useful candidate amino acid sequences of enzymes of the present inventions by SEQ ID NOS: 131-134 that are useful for increasing oxaloacetate production in a strains that would also carry the ODC and OPR enzymes of the present invention.

FIG. 16 shows Table 5 that lists the nucleotide sequences by SEQ ID NOS: 181-186 for various regulatory sequences used in making constructs for the expression of the various enzymes of the present invention in S. pombe.

### DETAILED DESCRIPTION OF THE INVENTION

The present disclosure arose from a project to genetically engineer a Schizosaccharomyces pombe yeast cell to ferment dextrose to 3-hydroxypropionic acid (3-HP). S. pombe is a yeast with high tolerance to low pH and to organic acids, making it an ideal host for engineering 3-HP production. 3-HP can therefore be produced in its protonated form with minimal pH adjustment and be isolated from the media thereafter. As used herein “high tolerance to low pH” or merely “acid tolerant” means an organism is capable of vegetative increase in biomass when grown in a media below pH 5.0 that is no less than 50% of the biomass accumulation the organism can obtain in a fermentation media having an optimal pH for vegetative growth of the organism.

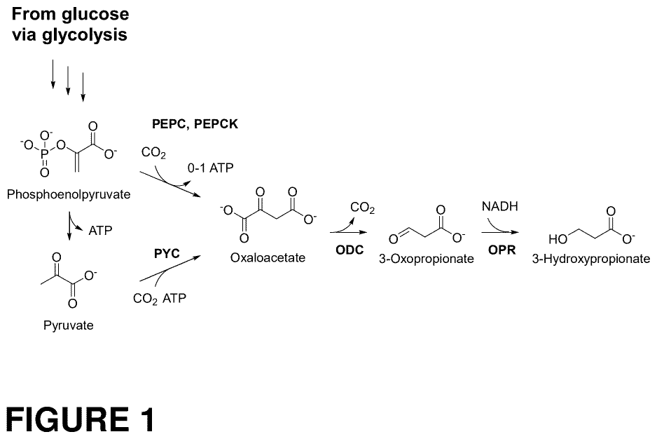

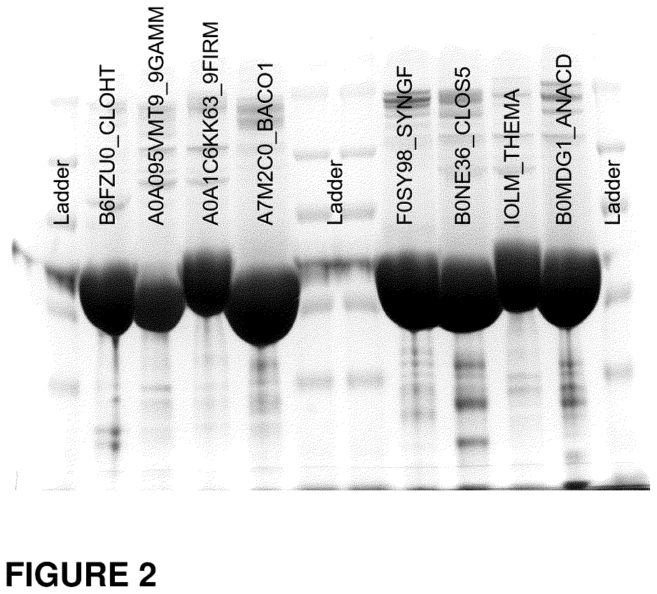

There is no known naturally occurring metabolic pathway for making 3-HP. The present invention recognizes that enzymes used for other naturally occurring metabolic pathways can exhibit promiscuity in substates use so that such enzymes may be recruited for use in a non-naturally occurring pathway for production of 3-HP in S, pombe or other acid tolerant yeast strains. FIG. 1 shows the non-natural enzymatic pathway for making 3-HP of the present invention that uses an enzyme exhibiting an oxaloacetate α-decarboxylase (ODC) activity, which is an activity that converts oxaloacetate to 3-oxopropionate (a.k.a malonyl semialdehyde) and uses an enzyme exhibiting a 3-oxopropionate reductase (OPR) activity, which is an activity that reduces 3-oxopropionate to give 3-HP. The production of 3-HP from glucose using this pathway is electron and ATP balanced, making it an attractive route from the perspective of theoretical molar yield. Neither ODC nor OPR activities are known to exist in nature, however, and must therefore be identified or engineered.

In addition to expression of ODC and OPR, additional modifications to S. pombe are desirable for the production of 3-HP at titers, rates, and yields necessary for industrial implementation. To provide oxaloacetate, one or more enzymes selected from the group of pyruvate carboxylase (PYC), a phosphoenolpyruvate carboxylase (PEPC), and/or phosphoenolpyruvate carboxykinase (PEPCK) can be engineered for expression or overexpression in S. pombe. In certain embodiments, a reduction of byproduct synthesis in the form of ethanol or glycerol may be achieved by knocking out ethanol biosynthesis pathway genes or by enhancing expression of naturally occurring alcohol dehydrogenase genes (e.g., ADH1, and/or ADH4) and or enhancing expression of naturally occurring pyruvate decarboxylase genes (PDC201) to reduce ethanol alone or in conjunction with overexpression of glycerol phosphate dehydrogenase genes (e.g., GPD1).

Using a bioinformatics computational program developed by Zymvol (Barcelona, Spain) that predicts structure function relationships of enzymes based on known activities, substrates, tertiary structure and amino acid sequences of over 200 million proteins available from UniProt. UniProt databases were mined to select candidate enzyme sequences predicted to exhibit OPR and ODC activity, as well as enzymes that would exhibit enhanced oxaloacetate production from phosphoenolpyruvate or pyruvate with a PEPC, PEPCK or PYC like activity. Enzyme sequences predicted to have such activity are provided herein by SEQ ID NOS: 1-186. The invention includes use of these sequences or functional derivative thereof. As used herein, a “functional derivative” is a protein sequence derived from the recited sequence that retains the enzymatic activity of the recited sequence but may contain silent mutations or mutations that alter other properties of the recited sequence such as thermal stability, pH tolerance or kinetic properties of the enzyme. In most cases, a functional derivative will be a sequence that minimally is at least 90%, or more typically at least 95% and still more typically at least 98% identical to the recited sequence.

### Screening Candidates for 3-Oxopropionate Reductases Activity

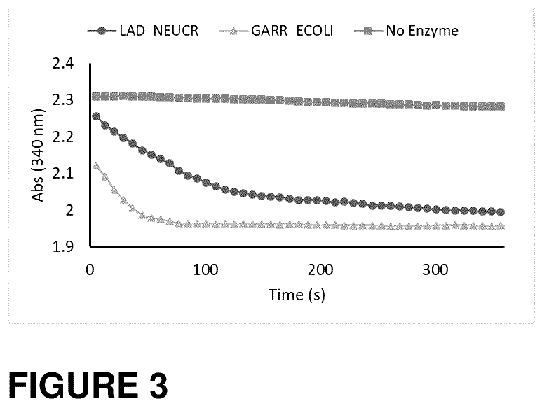

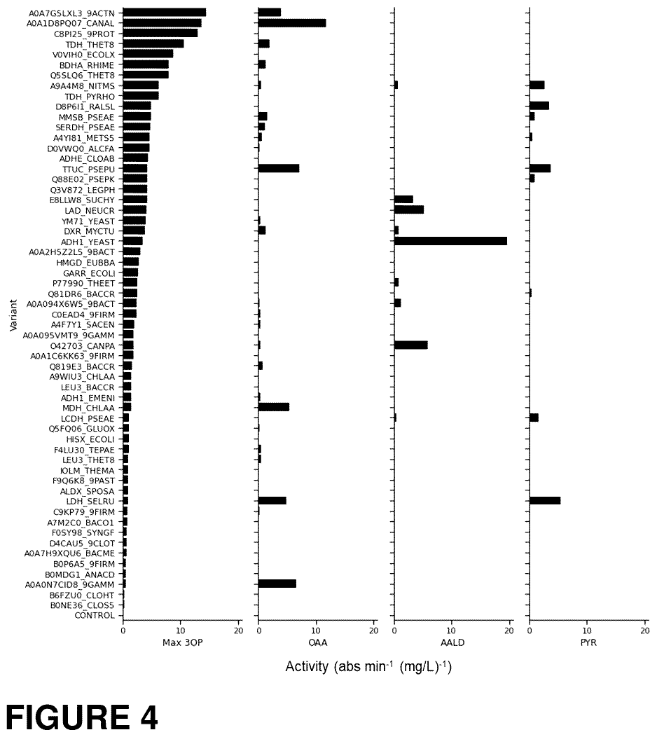

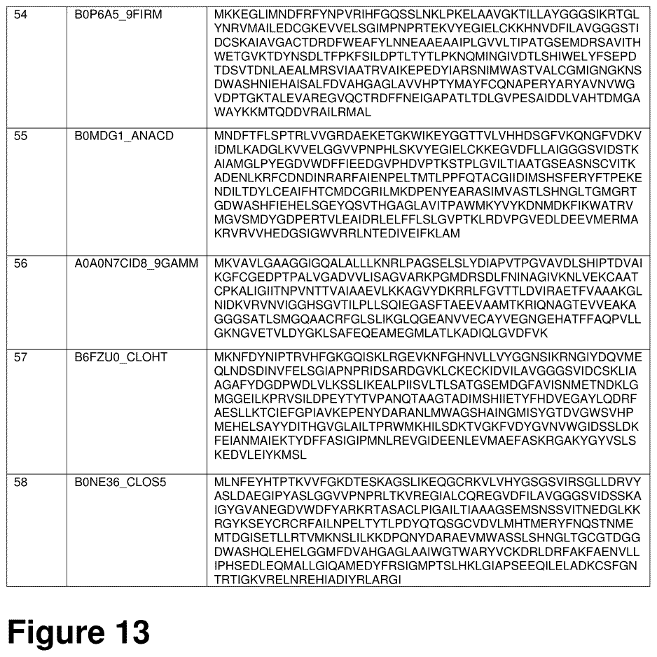

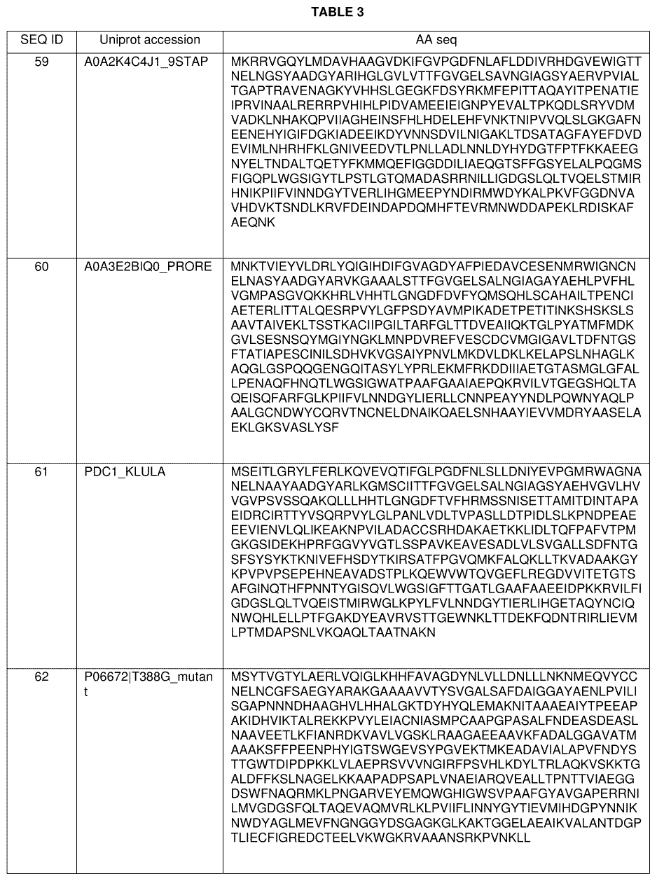

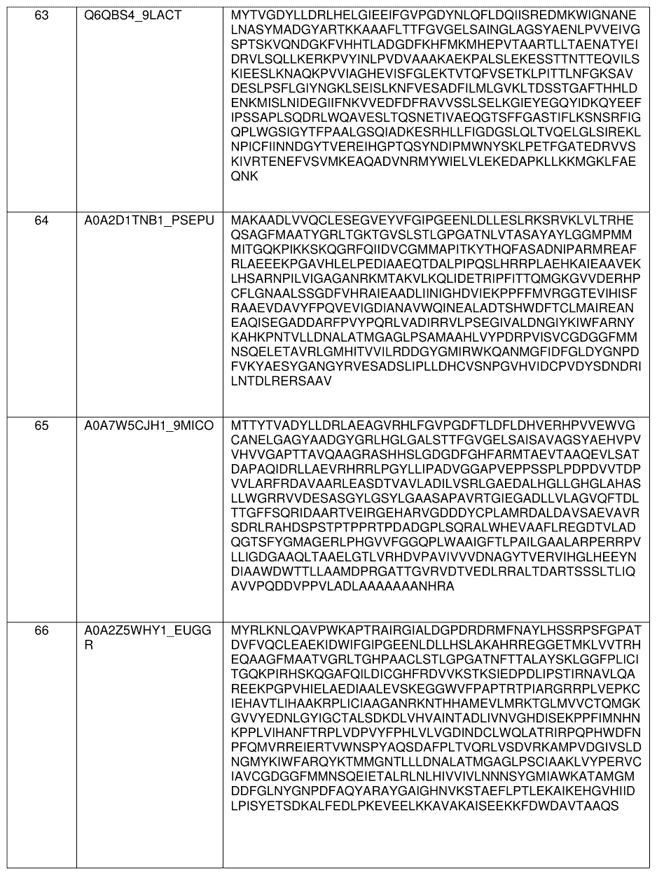

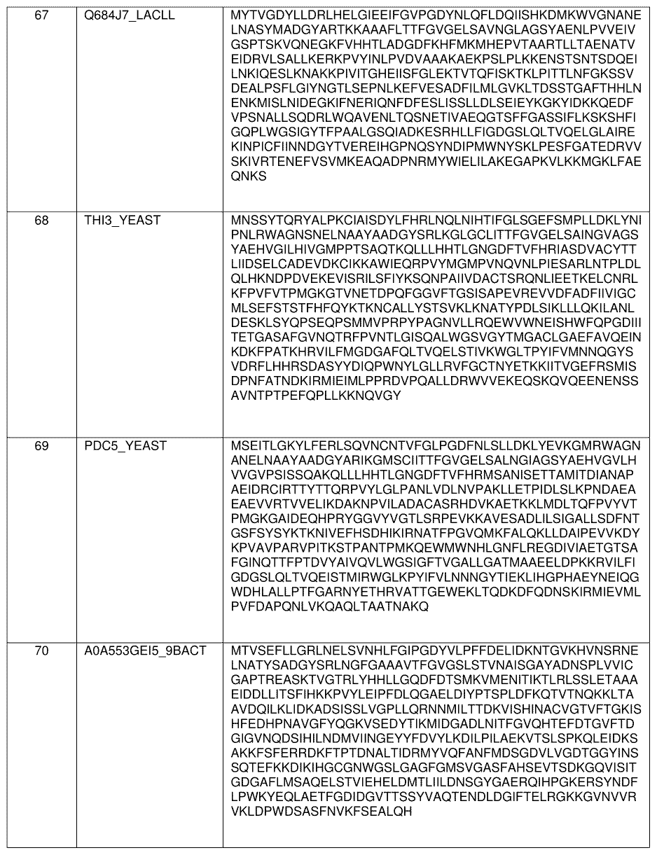

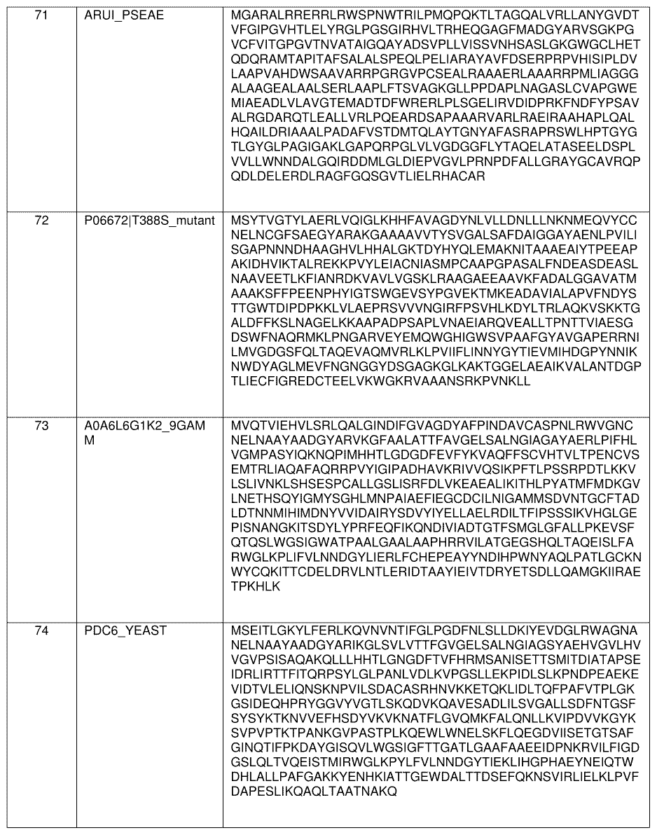

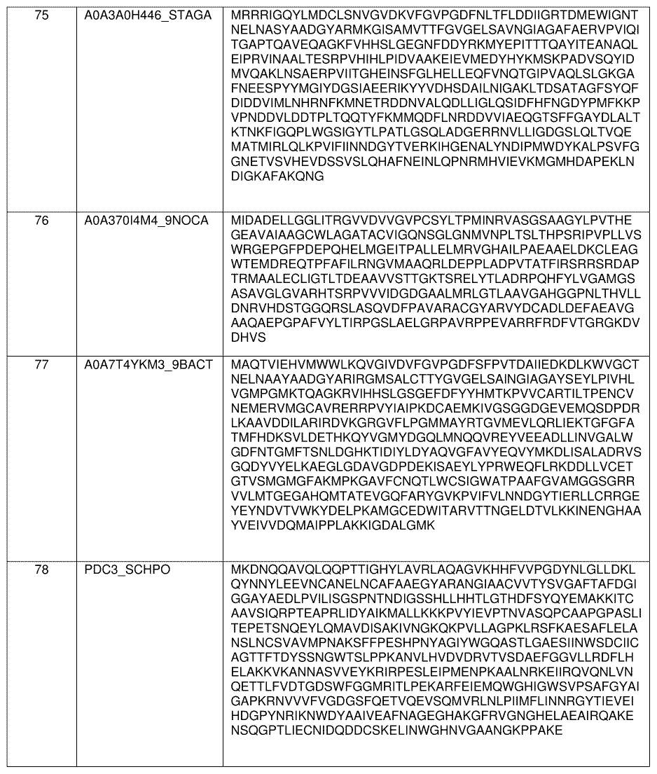

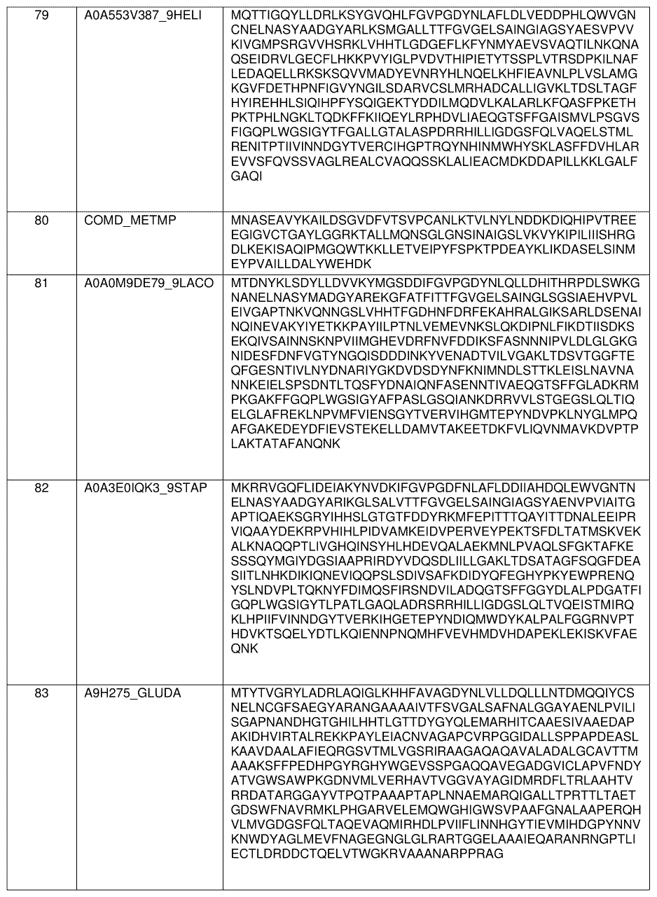

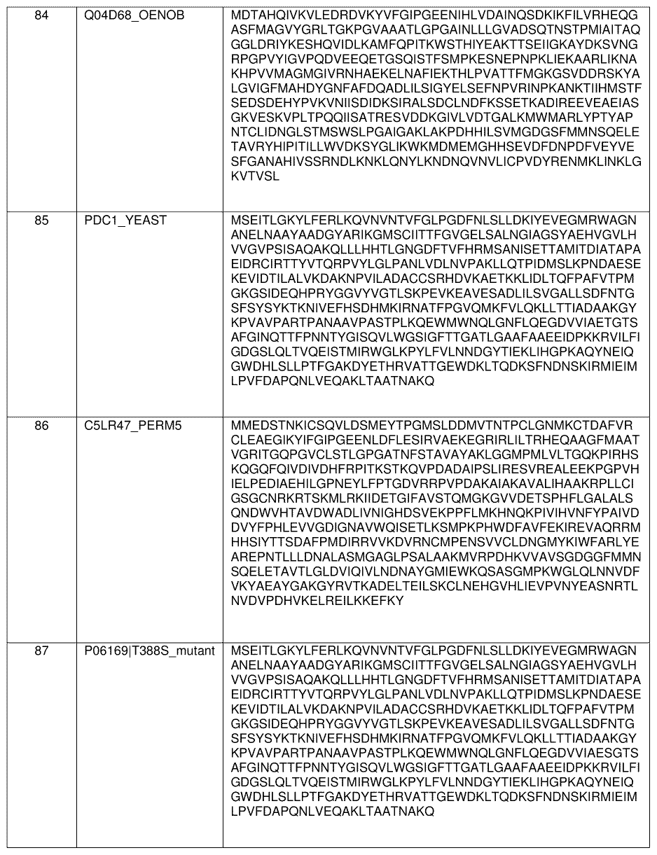

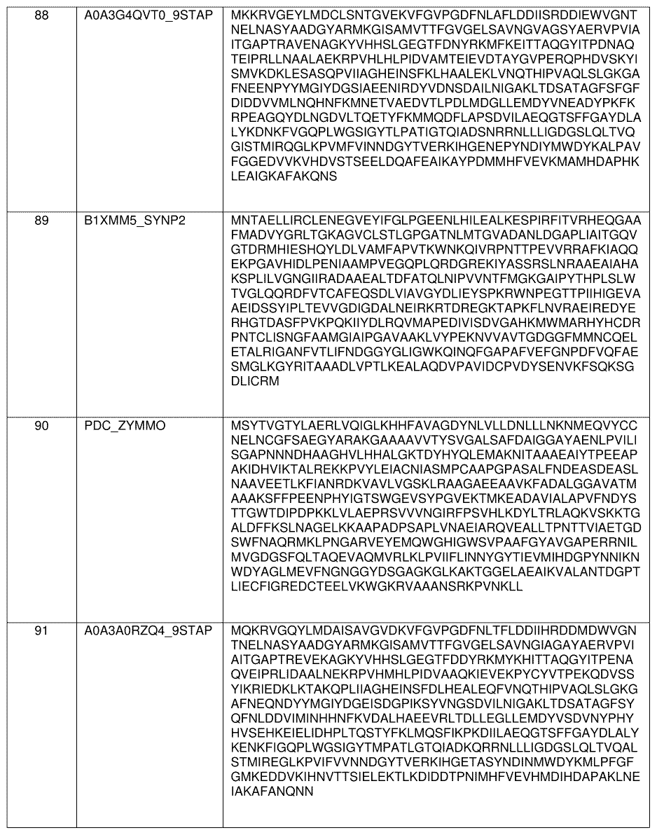

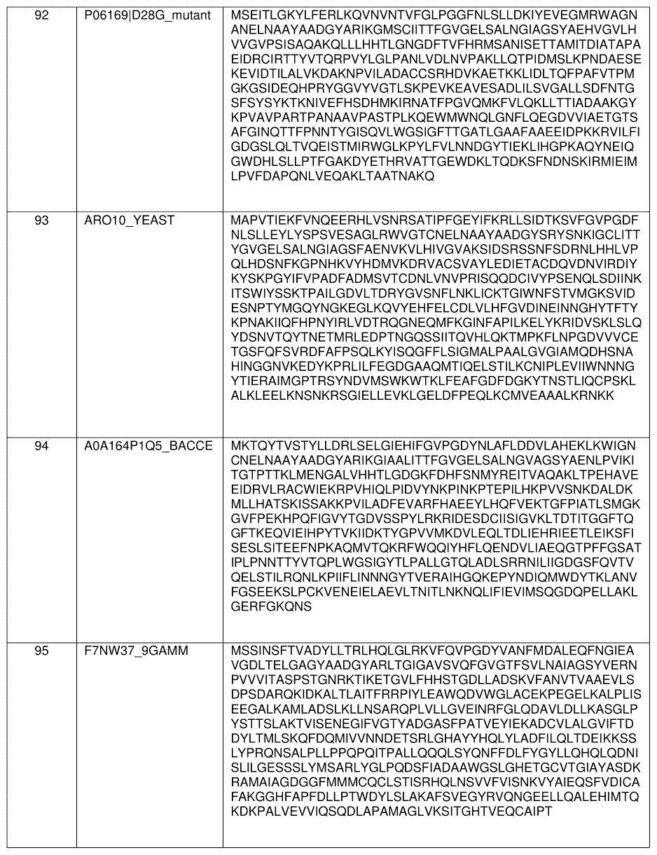

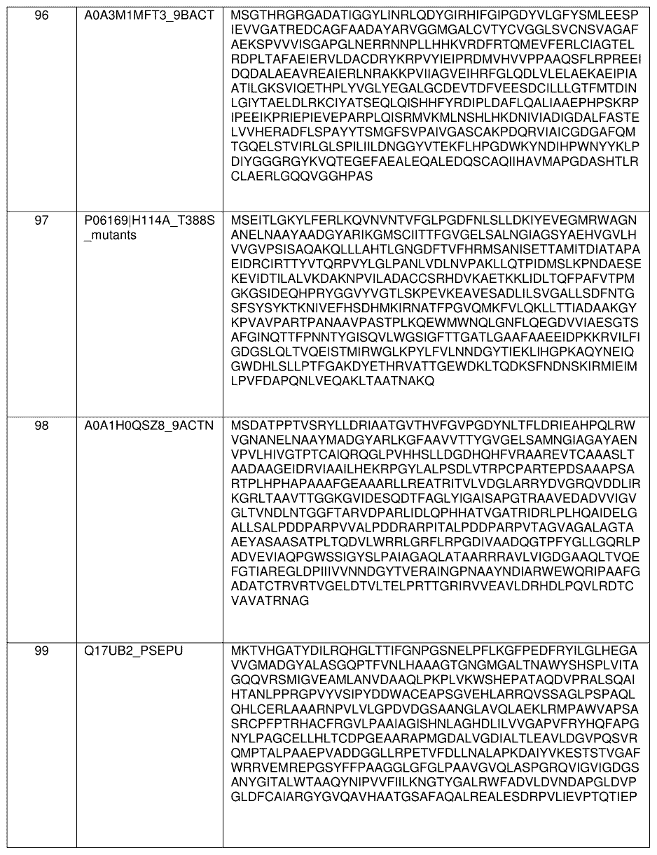

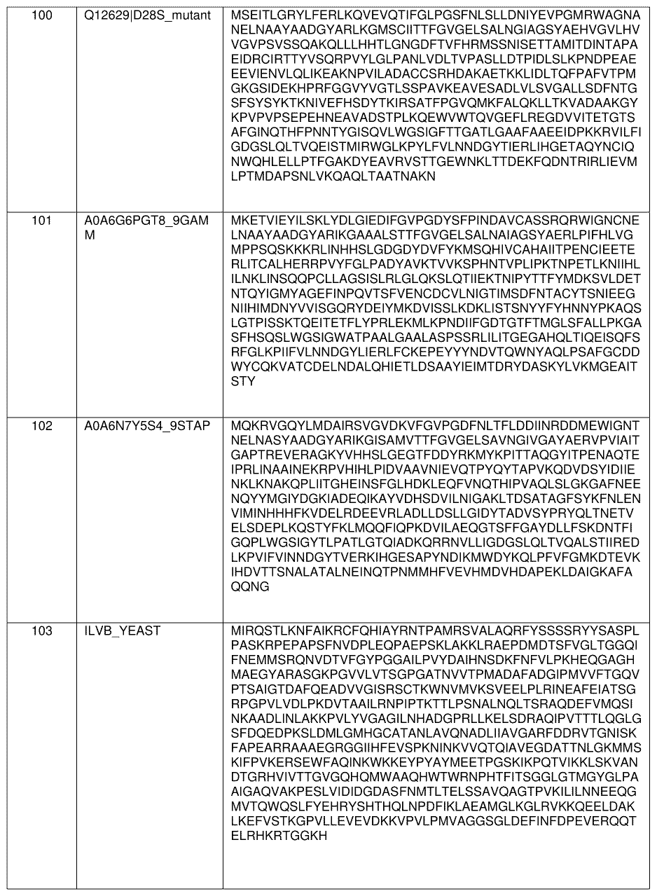

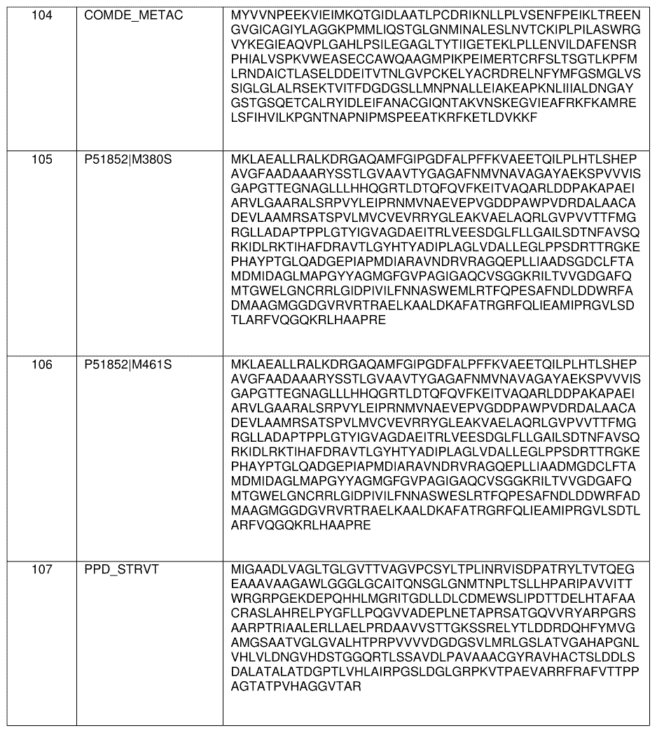

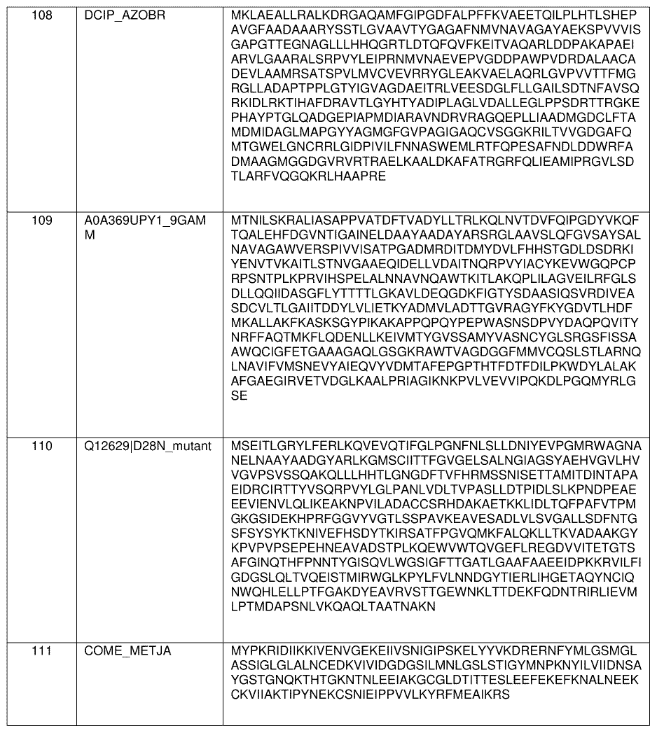

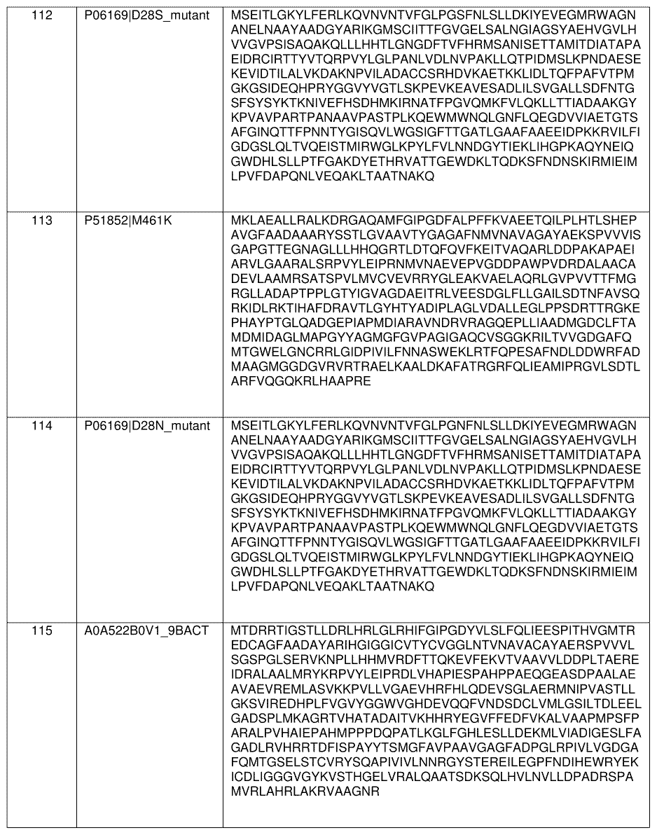

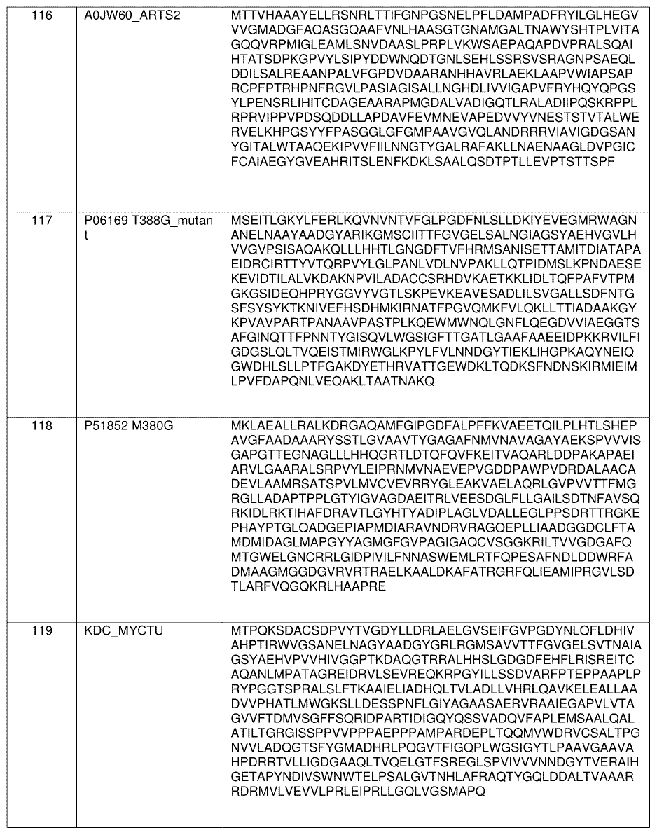

Candidate enzymes that were predicted to exhibit OPR activity were obtained by synthesizing genes encoding candidate enzymes (FIG. 13, Table 2) along with a histidine-tagged N-terminal peptide. The genes were expressed in E. coli BL21 (DE3), which was lysed and the candidate enzymes wee purified therefrom over nickel containing columns using conventional methods. Purified enzymes were desalted and buffer exchanged using Zeba desalting columns (ThermoFisher) into 100 mM KPi buffer pH 7.2. The purified enzymes were stored as aliquots at −80° C. When needed, frozen aliquots were thawed on ice and tested in a reaction mixture comprised of 100 mM KPi buffer pH 7.2, 1 mM NADH, and 2.5% of a freshly prepared crude preparation of 3-oxopropionate. To test promiscuity, 25 mM of either acetaldehyde, pyruvate, or oxaloacetate was used in place of 3-oxopropionate. 2 μL of purified OPR was used to initiate 100 μL reactions in a 96-well plate. NADH consumption was monitored at room temperature by absorbance at 340 nm as measured using a Biotek Synergy H1 plate reader (FIG. 3,4).

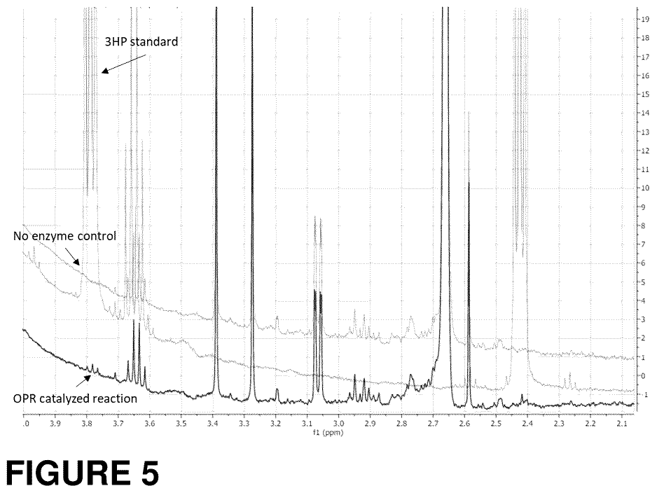

To confirm the desired reaction, a 600 μL reaction mixture comprised of 100 mM KPi buffer pH 7.2, 5 mM NADH, and 5% freshly prepared 3-oxopropionate was prepared and the reaction initiated by the addition of 5 μL of purified candidate OPR enzyme. The formation of 3-HP in the reaction was detected by 1H NMR (FIG. 5).

### Screening Candidates for Oxaloacetate Decarboxylases Activity

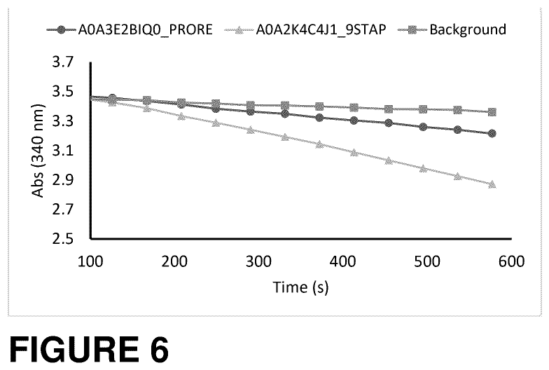

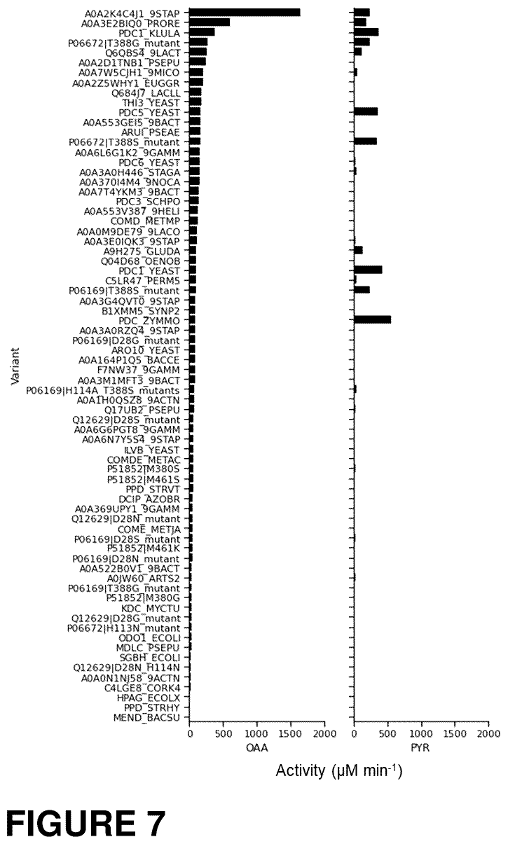

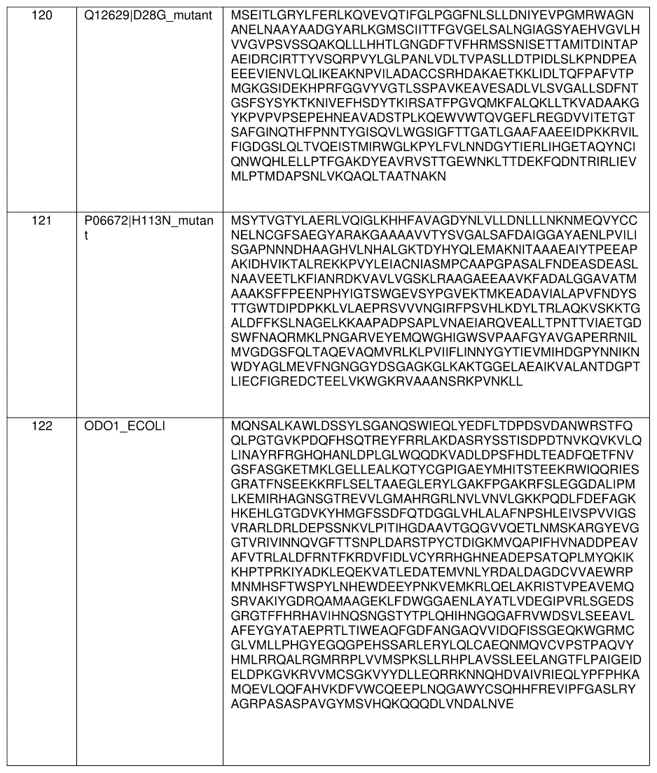

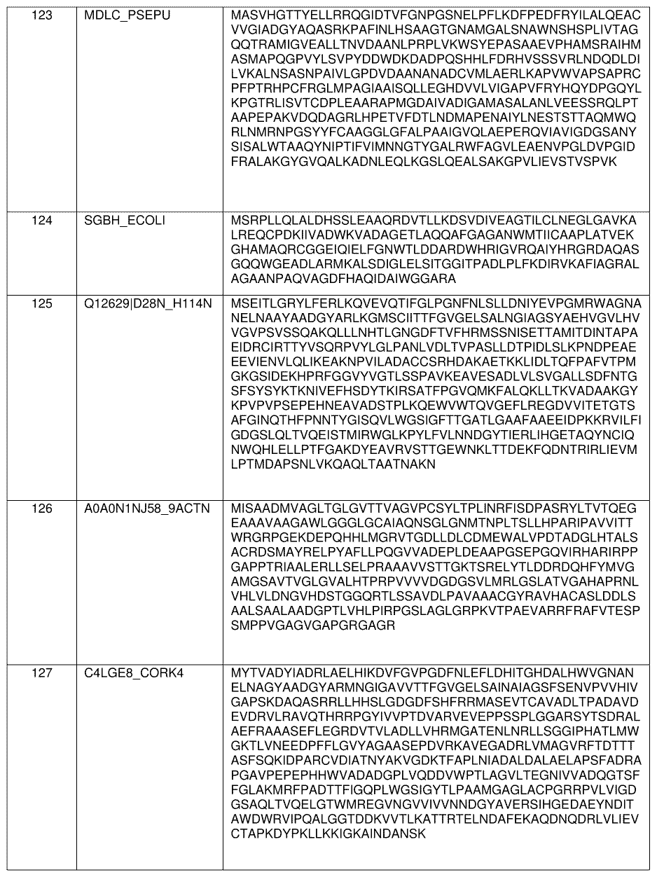

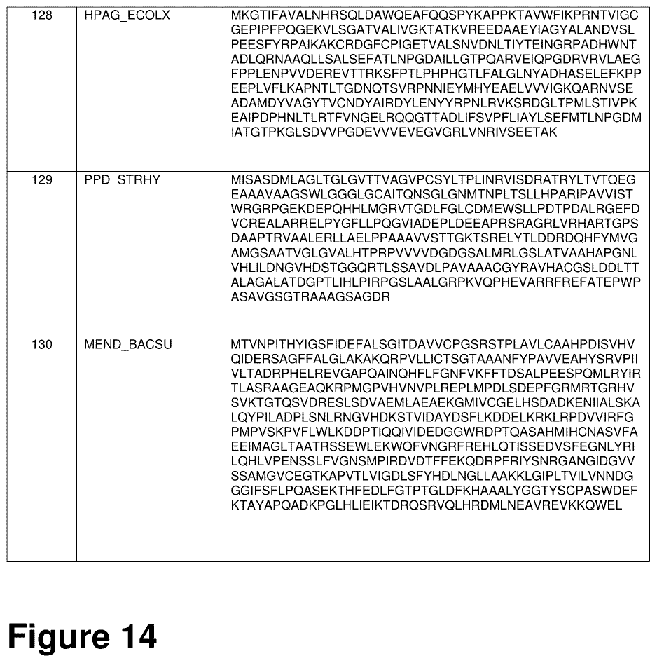

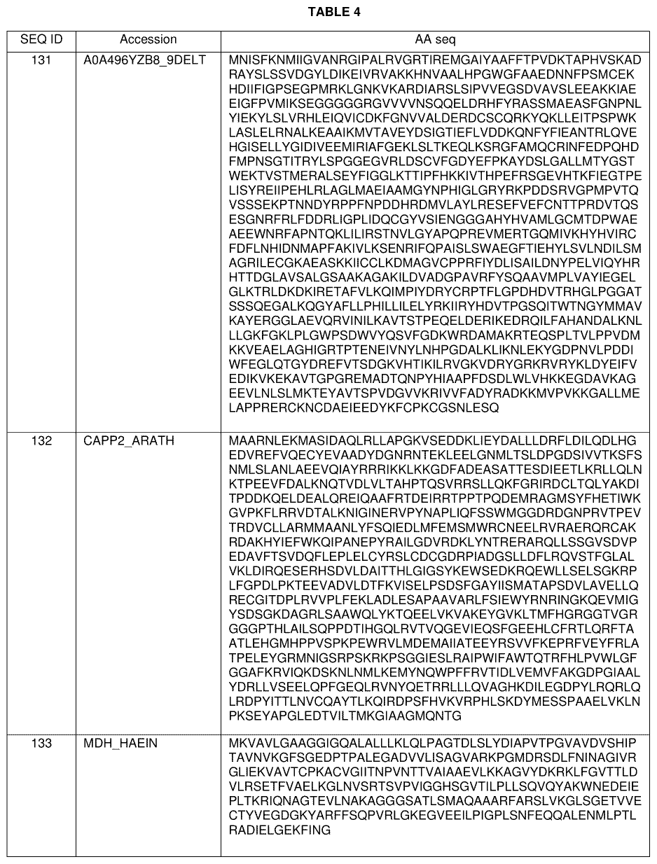

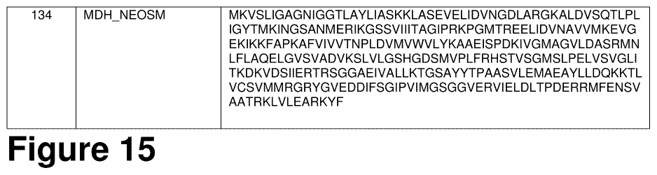

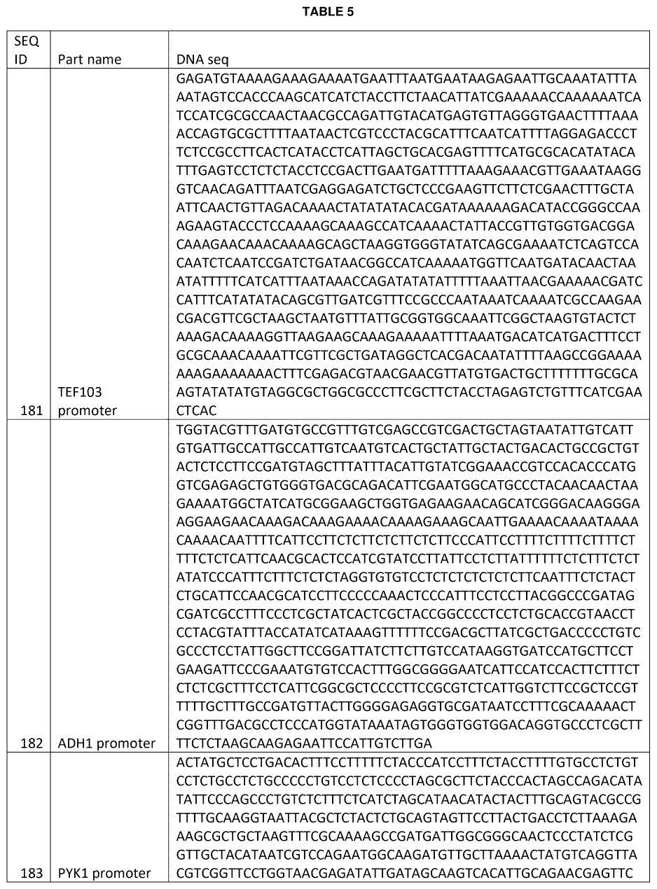

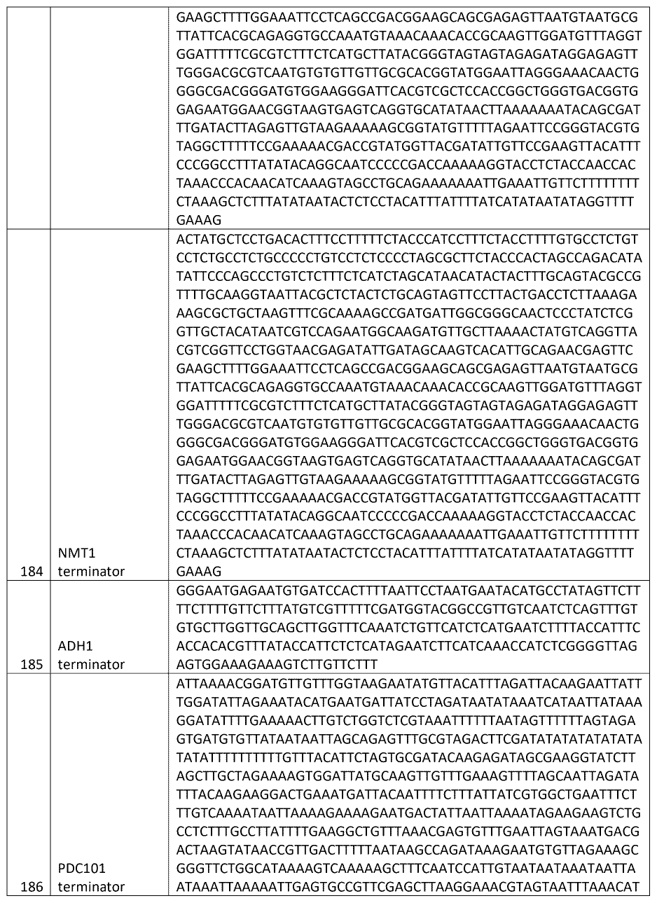

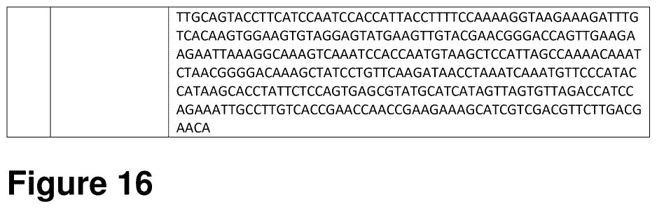

Candidate enzymes that were predicted to exhibit ODC activity were obtained by synthesizing genes encoding candidate enzymes (FIG. 14, Table 3), which were were expressed in and purified from E. coli BL21 (DE3) using the same methods for expression and purification of OPR candidates. Purified enzymes were desalted and buffer exchanged using Zeba desalting columns (ThermoFisher) into 100 mM KPi buffer pH 7.2, 0.1 mM TPP, and 1 mM MgCl2. The purified enzymes were stored as aliquots at −80° C. When needed, frozen aliquots were thawed on ice and tested in a reaction mixture comprised of 100 mM KPi buffer pH 7.2, 0.1 mM TPP, 1 mM MgCl2, 25 mM oxaloacetate, 2 mM NADH, and 0.3 g/L purified E. coli GarR (SEQ ID NO: 9). 55 μL reactions were initiated by addition of 5 μL of purified enzyme in a half-area 96-well plate. NADH consumption was monitored at room temperature by absorbance at 340 nm as measured using a Biotek Synergy H1 plate reader (FIG. 6, 7).

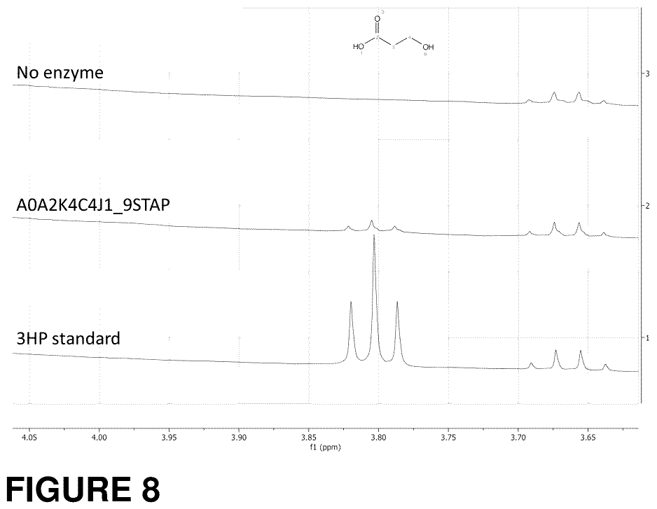

The conversion of oxaloacetate to 3-HP was demonstrated in a 500 μL reaction mixture comprised of 100 mM KPi buffer pH 7.2, 0.1 mM TPP, 1 mM MgCl2, 25 mM oxaloacetate, 5 mM NADH, and 0.15 g/L purified E. coli GarR. The reactions were initiated by addition of 15 μL of purified ODC (A0A2K4C4J1_9STAP, SEQ ID NO: 74). The formation of 3-HP in the reaction was detected by ¹H NMR (FIG. 8).

Expression of ODC and OPR in S. pombe

The remainder of the present disclosure describes the construction and use of certain genetic constructs for the expression of ODC and OPR (and other genes) in S. pombe. Table 1 below is a nomenclature reference to understand the meaning of certain symbols used herein to describe a given genetic construct.

TABLE 1

| | | | Example Genotype |
|---|---|---|---|
| Symbol | Meaning | Example | Description |
| Superscript (+) | Functional | ADH1⁺ | The ADH1 gene is |
| with capital | gene | | wild type and |
| letters | | | functional |
| Δ after gene name | Gene | adh1Δ | ADH1 is deleted and |
| | deletion | | non-functional |
| :: between two | Gene | adh1Δ::LDH | LDH is inserted at |
| genes | insertion | | ADH1 and ADH1 is |
| | | | deleted (and non- |
| | | | functional) |
| P with subscript | Promoter | P_{TDH3 -}LcLDH | The promoter of the |
| capital letters | | | TDH3 gene is located |
| before normal | | | upstream of the |
| case capital letters | | | LcLDH gene and |
| for a designated | | | drives its expression |
| gene | | | |
| T with subscript | Terminator | LcLDH-T_{ADH1} | The terminator of the |
| capital letters | | | ADH1 gene is located |
| after normal case | | | downstream of he |
| capital letters for | | | LcLDH gene to |
| a designated gene | | | terminate |
| | | | transcription |

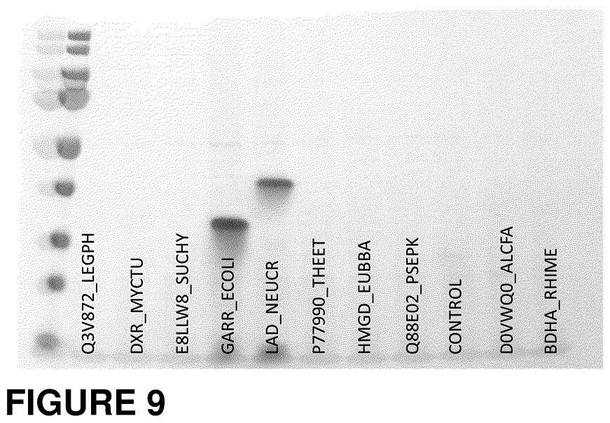

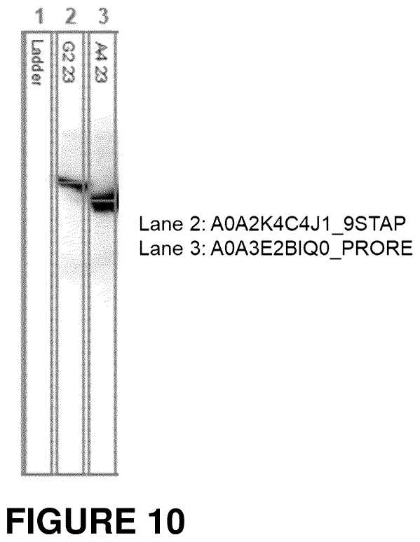

Codon optimized genes encoding prospective his-tagged ODCs and OPRs were synthesized in plasmids for expression in S. pombe under control of the TEF103 promoter (SEQ ID NO: 181) and the ADH1 terminator (SEQ ID NO: 185). The plasmids were transformed into S. pombe strain NCYC936 and transformants were selected for on 5 g/L yeast extract, 0.8 g/L complete supplement mixture (CSM), 30 g/L dextrose agar plates containing 50 mg/L G418. Transformants were further grown in liquid media containing 5 g/L yeast extract, 0.8 g/L complete supplement mixture (CSM), 30 g/L dextrose and 100 mg/L G418. S. pombe cell pellets were harvested after 24 hours of growth in liquid media by centrifugation, washed once with 100 mM KPi buffer pH 7.4, and frozen. Frozen S. pombe cell pellets were thawed and lysed in 100 mM KPi buffer pH 7.4 and 1 g/L Zymolyase 20T by incubation at 37° C. for one hour. Cell lysates were analyzed by SDS-PAGE using a NuPAGE 10% Bis-Tris precast gel following the manufacturer's instructions. Expressed protein was detected by Western blotting using the ThermoFisher iBlot2 and iBind Flex systems with a 6×-His tag mouse monoclonal primary antibody (Invitrogen MA1-21315) and goat anti-mouse alkaline phosphate conjugate secondary antibody (Invitrogen G21060) with either chemiluminescent (Invitrogen WP20002) or colorimetric (Thermo Scientific 34042) alkaline phosphate substrate for detection of the expression of his-tagged ODCs and OPRs (FIGS. 9 and 10).

### Screening Enzymes for Production of Oxaloacetate

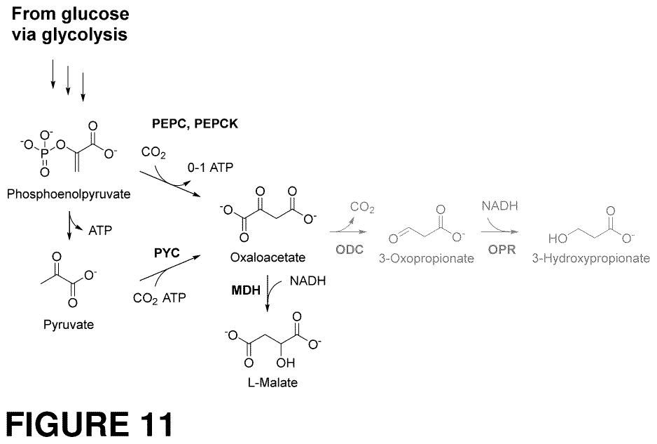

Enzymes capable of increasing the pool of the intermediate oxaloacetate were screened by their ability to support malic acid production in S. pombe by expression of a malate dehydrogenase (MDH) thereby using malate production as an in-vivo assay for increased oxaloacetate production by the enzymatic pathway shown in FIG. 11. An engineered strain of S. pombe NCYC936 from the British National Collection of Yeast Cultures that was previously developed for the production of L-lactic acid (L-2-hydroxypropionic acid) described in U.S. provisional application No. 63/224,408 having the genotype pdc201::P_{ACT1}-LcLDH ura4Δ::BC4241 adh1Δ::P_{ACT1}-LcLDH adh4Δ::BC59 gpd1Δ::URA4 and having been evolved for improved growth was used as the host strain for expression of MDH and candidate oxaloacetate enhancing enzymes. This strain has reduced ability to synthesize the byproducts glycerol and ethanol, arising from deletions of the adh1, adh4, pdc201, and gpd genes from the strain. To this strain, URA4 previously introduced at the gpd1 locus was replaced with a noncoding sequence (barcode, BC). URA4 was then reinserted at the mae2 locus, deleting mae2, which encodes malic enzyme and is responsible for the consumption of malate. The URA4 at mae2 was in turn removed and replaced with a barcode sequence, and URA4 was reinserted at the pdc201 locus, deleting the P_{ACT1}-LcLDH construct. This strain NCYC936 has the genotype pdc201::URA4 ura4Δ::BC4241 adh1Δ::P_{ACT1}-LcLDH adh4Δ::BC59 gpd1Δ::BC mae2Δ::BC3579 and served as the basis for further engineering.

To screen for malate (and therefore increased oxaloacetate) production, a combinatorial library of DNA was designed to express an oxaloacetate forming enzyme and a malate dehydrogenase. Oxaloacetate forming enzymes were selected by a search of the UniProt database for the EC numbers 6.4.1.1 and 4.1.1.31, corresponding to the reactions catalyzed by pyruvate carboxylase (PYC) and phosphoenolpyruvate carboxylase (PEPC), respectively. Similarly, MDHs were selected by a search of the UniProt database for the EC number 1.1.1.37, corresponding to the NADH-dependent reduction of oxaloacetate to malate. Fifty of each of oxaloacetate forming enzymes (25 PYC and 25 PEPC) and MDH were selected for the combinatorial library. Two examples of oxaloacetate forming enzymes are PYC according to SEQ ID NO: 131 and PEPC according to SEQ ID NO: 132. Two examples of MDH enzymes are SEQ ID NO: 133 and SEQ ID NO: 134.

DNA was then assembled to enable the expression of PYC/PEPC and MDH simultaneously in S. pombe. MDHs were expressed using the ADH1 promoter (SEQ ID NO: 182) and NMT1 terminator (SEQ ID NO: 184). PYCs and PEPCs were expressed using the PYK1 promoter (SEQ ID NO: 183) and ADH1 terminator (SEQ ID NO: 185). When assembled, these DNA constructs had the construct layout: P_{ADH1}-MDH-T_{NMT1}-P_{PYK1}-PYC/PEPC-T¬_{ADH1}, wherein the ADH1 promoter and ADH1 terminator also serve as the homology sequences enabling recombination at the adh1 locus. Transformation of the above S. pombe host strain with the DNA construct results in replacement of the P¬_{ACT1}¬-LcLDH construct at adh1 with the desired DNA sequence for expressing MDH and PYC or PEPC.

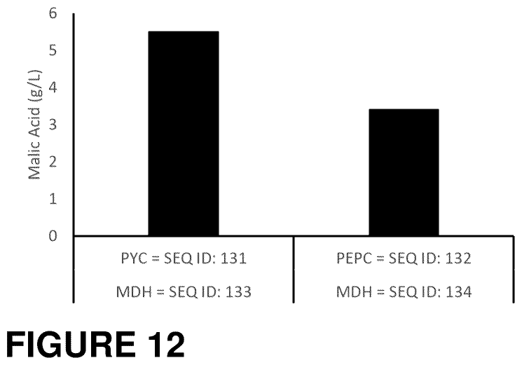

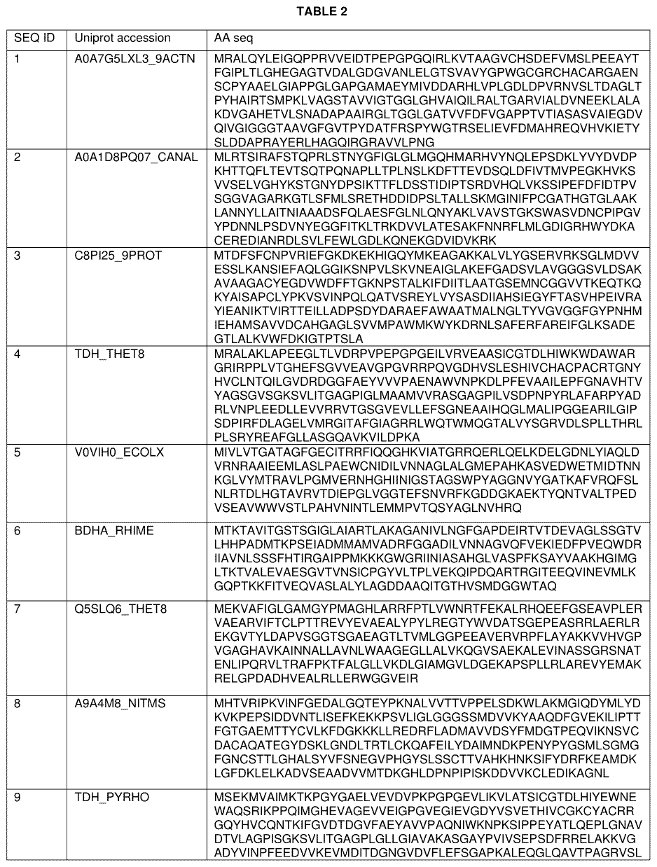

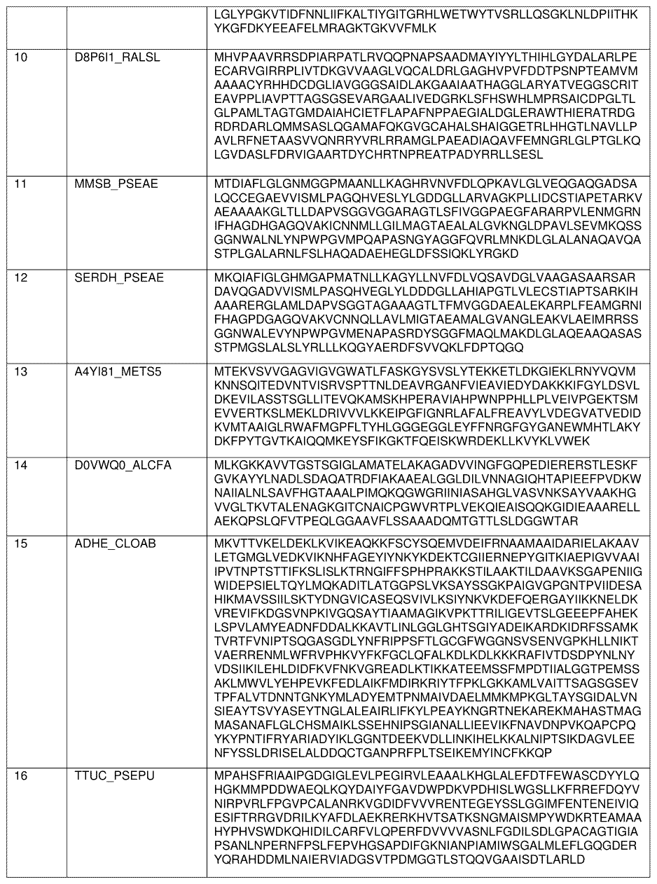

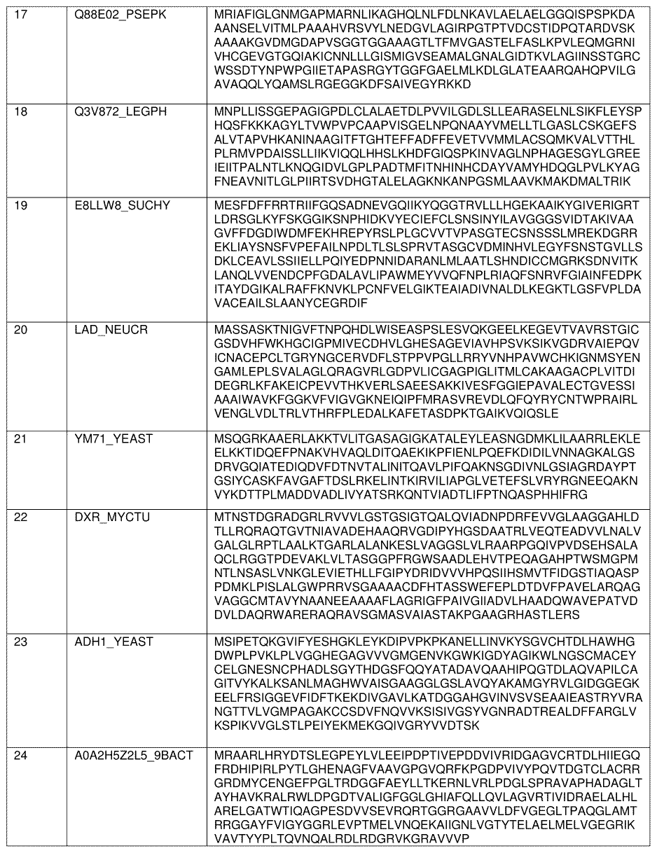

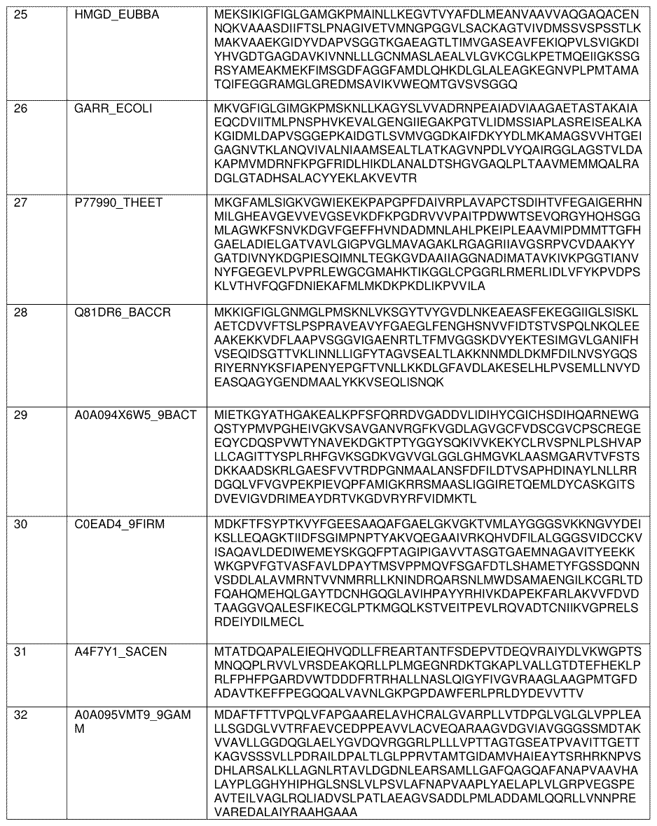

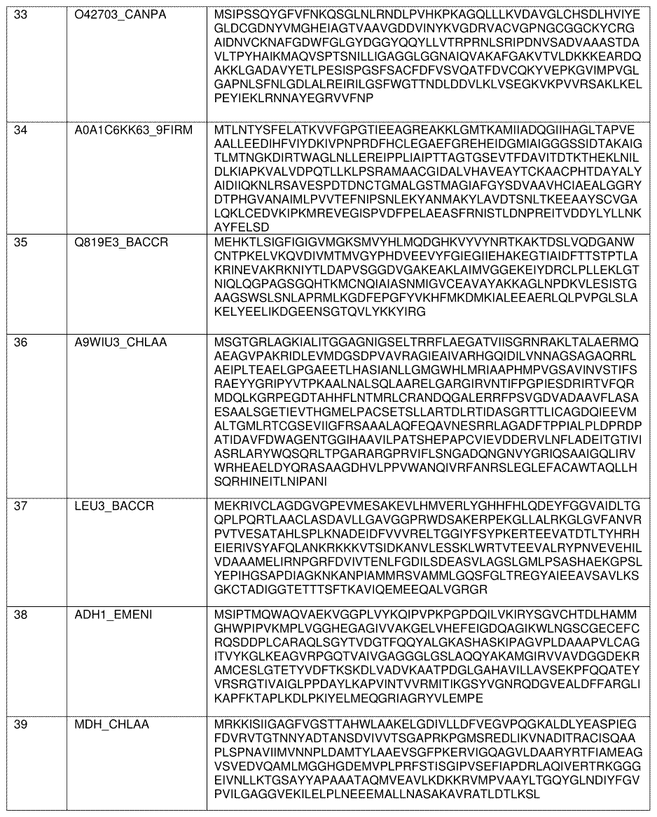

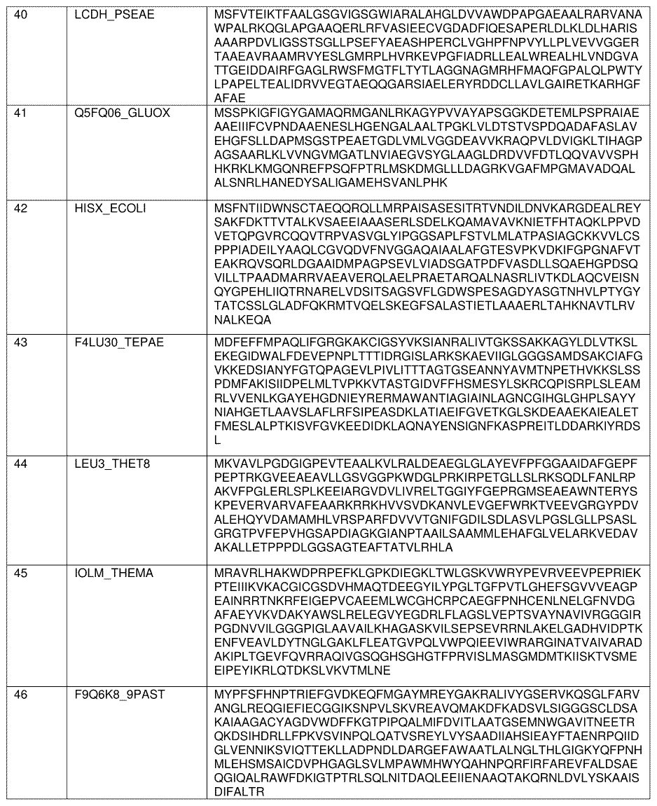

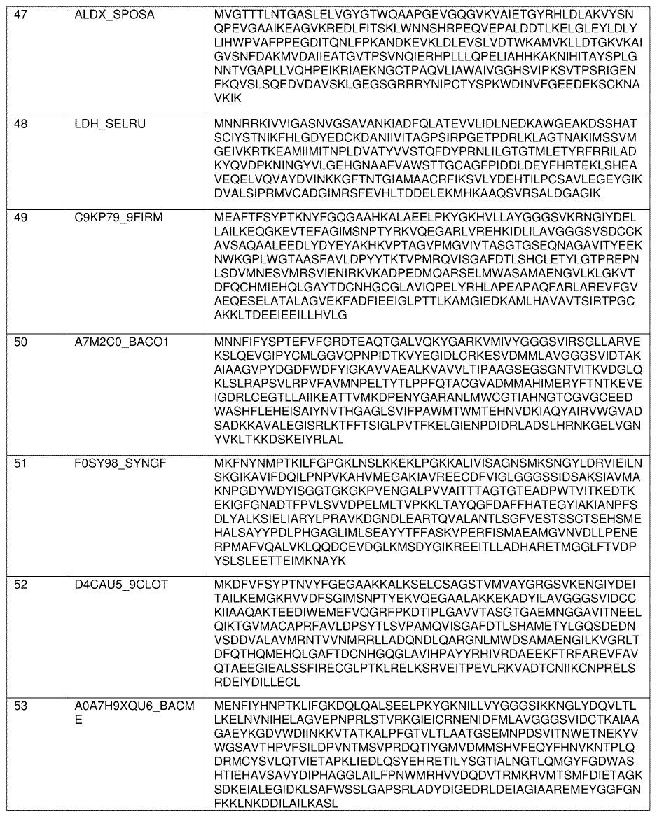

The resulting transformants of S. pombe were screened for malic acid production in a 1.1 mL 96-well plate containing 440 μL of media comprised of 15 g/L Roquette corn steep liquor (CSL), 50 g/L dextrose, pH adjusted to 4.5 and filter sterilized. Plates were incubated at 33° C. at 900 rpm and 80% humidity and analyzed after 24 or 48 hours for malic acid production by HPLC-RID. FIG. 12 shows that stains expressing either an exemplary PYC or PEPC in conjunction with two different exemplary MDHs had increased production of malic acid indicating increased production of oxaloacetate.

### Engineered S. Pombe Producing 3-HP at Low pH

A combinatorial DNA library is created, wherein OPR is expressed using the ADH1 promoter and NMT1 terminator, ODC is expressed under the TEF103 promoter and PDC101 terminator (SEQ ID NO: 186), and PYC or PEPC is expressed from the PYK1 promoter and ADH1 terminator. The fully assembled DNA has the layout: P_{ADH1}-OPR-T_{NMT1}-P_{TEF103}-ODC-TP_{DC101}-P_{PYK1}-PYC/PEPC-T_{ADH1} and is transformed into the S. pombe host strain having the genotype NCYC936 pdc201::URA4 ura4Δ::BC4241 adh1Δ::PACT1-LcLDH adh4Δ::BC59 gpd1Δ::BC mae2Δ::BC3579. The transformed DNA recombines to replace P_{ACT1}-LcLDH at the adh1 locus. The resulting transformants are grown in a suitable media with dextrose as the primary substrate, for example comprised of 15 g/L corn steep liquor (CSL) and 50 g/L dextrose. The culture is incubated with shaking at 33° C. for up to 120 hours. The fermentation media is analyzed for 3-HP by HPLC and is found to contain between 0.1 to 10 g/L 3-HP. The final pH of the media is less than 4.

1. An acid tolerant yeast strain comprising nucleotide sequences that include a promoter sequence operable in the microorganism operably linked to coding sequences that encode non-native enzymes that exhibit a) an oxaloacetate decarboxylase (ODC) activity that converts oxaloacetate to 3-oxopropionate; and b) a 3-oxopropionate reductase (OPR) activity that converts the 3-opropionate to 3-hydroxypropianate wherein the enzyme that exhibits an OPR activity has a protein sequence selected from the group consisting of SEQ ID NOS: 1-58 or functional derivative thereof.

2. The acid tolerant yeast strain of claim 1, wherein the enzyme that exhibits an OPR activity has a protein sequence selected from the group consisting of SEQ ID NOS: 1-20 or functional derivative thereof.

3. The acid tolerant yeast strain of claim 1, wherein the enzyme that exhibits an OPR activity has a protein sequence selected from the group consisting of SEQ ID NOS: 1-5 or functional derivative thereof.

4. The acid tolerant yeast strain of claim 1, wherein the enzyme that exhibits an OPR activity has a protein sequence selected from SEQ ID NOS: 1 or 3 or functional derivative thereof.

5. The acid tolerant yeast strain of claim 1, wherein the enzyme that exhibits an ODC activity has a protein sequence selected from the group consisting of SEQ ID NOS: 59-130 or functional derivative thereof.

6. The acid tolerant yeast strain of claim 1, wherein the enzyme that exhibits an ODC activity has a protein sequence selected from the group consisting of SEQ ID NOS: 59-65 or functional derivative thereof.

7. The acid tolerant yeast strain of claim 1, wherein the enzyme that exhibits an ODC activity has a protein sequence according to SEQ ID NOS: 59-60 or functional derivative thereof.

8. The acid tolerant yeast strain of claim 1, wherein the acid tolerant yeast strain further includes a nucleotide sequence that encodes an enzyme that exhibits at least one of a pyruvate carboxylase activity, a phosphoenolpyruvate carboxy kinase activity, and a phosphoenolpyruvate carboxylase activity operably linked to a non-native promoter that expresses the enzyme in the acid tolerant yeast strain.

9. The acid tolerant yeast strain of claim 8 wherein the enzyme exhibits a phosphoenolpyruvate carboxylase activity or a phosphoenolpyruvate carboxylase according to SEQ ID NO: 132, or functional derivative thereof.

10. The acid tolerant yeast strain of claim 1, wherein the acid tolerant yeast is a strain of Schizosaccharomyces pombe.

11. An acid tolerant yeast strain comprising a nucleotide sequences that include a promoter sequence operable in the microorganism operably linked to coding sequences that encode non-native enzymes that exhibit a) an oxaloacetate decarboxylase (ODC) activity that converts oxaloacetate to 3-oxopropionate, wherein the ODC activity is provided by an enzyme having a sequence selected from the group consisting of SEQ ID NOS: 59-130 or functional derivative thereof; and b) a 3-oxopropionate reductase (OPR) activity that converts the 3-opropionate to 3-hydroxypropianate.

12. The acid tolerant yeast strain of claim 11, wherein the enzyme that exhibits an OPR activity has a protein sequence selected from the group consisting of SEQ ID NOS: 1-58 or functional derivative thereof.

13. The acid tolerant yeast of claim 11, wherein the enzyme that exhibits an OPR activity has a protein sequence selected from the group consisting of SEQ ID NOS: 1-20 or functional derivative thereof.

14. The acid tolerant yeast strain of claim 11, wherein the enzyme that exhibits an OPR activity has a protein sequence selected from the group consisting of SEQ ID NOS: 1-4 or functional derivative thereof.

15. The acid tolerant yeast train of claim 11, wherein the enzyme that exhibits an ODC activity has a protein sequence selected from the group consisting of SEQ ID NOS: 59-65 or functional derivative thereof.

16. The acid tolerant yeast strain of claim 11, wherein the enzyme that exhibits an ODC activity has a protein sequence according to SEQ ID NOS: 59 or functional derivative thereof.

17. The acid tolerant yeast strain of claim 11, wherein the acid tolerant yeast strain further includes a nucleotide sequence that encodes an enzyme that exhibits at least one of a pyruvate carboxylase activity, a phosphoenolpyruvate carboxy kinase activity, and a phosphoenolpyruvate carboxylase activity operably linked to a non-native promoter that expresses the enzyme in the acid tolerant yeast strain.

18. The acid tolerant yeast strain of claim 17 wherein the enzyme that exhibits at least one of a pyruvate carboxylase activity, a phosphoenolpyruvate carboxy kinase activity and a phosphoenolpyruvate carboxylase is at least one of a pyruvate carboxylase according to SEQ ID NO: 131 or a phosphoenolpyruvate carboxylase according to SEQ ID NO: 132, or functional derivative thereof.

19. The acid tolerant yeast strain of claim 11, wherein the acid tolerant yeast is a strain of Schizosaccharomyces pombe.

20. A method of determining whether a candidate enzyme increases oxaloacetate production in a microorganism comprising, expressing the candidate enzyme in the microorganism while simultaneously expressing an exogenous malate dehydrogenase gene in the microorganism and measuring the production of malic acid in the microorganism wherein an increase of malic acid production is determinative of whether the candidate enzyme increases oxaloacetate production in the microorganism.


SEQUENCE LISTING (186 sequences)

SEQ ID NO: 1 - protein, 346 aa, synthetic construct
MRALQYLEIGQPPRVVEIDTPEPGPGQIRLKVTAAGVCHSDEFVMSLPEEAYTFGIPLTLGHEGAGTVDALGDGVANLELGTSVAVYGPWGCGRCHACARGAENSCPYAAELGIAPPGLGAPGAMAEYMIVDDARHLVPLGDLDPVRNVSLTDAGLTPYHAIRTSMPKLVAGSTAVVIGTGGLGHVAIQILRALTGARVIALDVNEEKLALAKDVGAHETVLSNADAPAAIRGLTGGLGATVVFDFVGAPPTVTIASASVAIEGDVQIVGIGGGTAAVGFGVTPYDATFRSPYWGTRSELIEVFDMAHREQVHVKIETYSLDDAPRAYERLHAGQIRGRAVVLPNG

SEQ ID NO: 2 - protein, 354 aa, synthetic construct
MLRTSIRAFSTQPRLSTNYGFIGLGLMGQHMARHVYNQLEPSDKLYVYDVDPKHTTQFLTEVTSQTPQNAPLLTPLNSLKDFTTEVDSQLDFIVTMVPEGKHVKSVVSELVGHYKSTGNYDPSIKTTFLDSSTIDIPTSRDVHQLVKSSIPEFDFIDTPVSGGVAGARKGTLSFMLSRETHDDIDPSLTALLSKMGINIFPCGATHGTGLAAKLANNYLLAITNIAAADSFQLAESFGLNLQNYAKLVAVSTGKSWASVDNCPIPGVYPDNNLPSDVNYEGGFITKLTRKDVVLATESAKFNNRFLMLGDIGRHWYDKACEREDIANRDLSVLFEWLGDLKQNEKGDVIDVKRK

SEQ ID NO: 3 - protein, 339 aa, synthetic construct
MTDFSFCNPVRIEFGKDKEKHIGQYMKEAGAKKALVLYGSERVRKSGLMDVVESSLKANSIEFAQLGGIKSNPVLSKVNEAIGLAKEFGADSVLAVGGGSVLDSAKAVAAGACYEGDVWDFFTGKNPSTALKIFDIITLAATGSEMNCGGVVTKEQTKQKYAISAPCLYPKVSVINPQLQATVSREYLVYSASDIIAHSIEGYFTASVHPEIVRAYIEANIKTVIRTTEILLADPSDYDARAEFAWAATMALNGLTYVGVGGFGYPNHMIEHAMSAVVDCAHGAGLSVVMPAWMKWYKDRNLSAFERFAREIFGLKSADEGTLALKVWFDKIGTPTSLA

SEQ ID NO: 4 - protein, 343 aa, synthetic construct
MRALAKLAPEEGLTLVDRPVPEPGPGEILVRVEAASICGTDLHIWKWDAWARGRIRPPLVTGHEFSGVVEAVGPGVRRPQVGDHVSLESHIVCHACPACRTGNYHVCLNTQILGVDRDGGFAEYVVVPAENAWVNPKDLPFEVAAILEPFGNAVHTVYAGSGVSGKSVLITGAGPIGLMAAMVVRASGAGPILVSDPNPYRLAFARPYADRLVNPLEEDLLEVVRRVTGSGVEVLLEFSGNEAAIHQGLMALIPGGEARILGIPSDPIRFDLAGELVMRGITAFGIAGRRLWQTWMQGTALVYSGRVDLSPLLTHRLPLSRYREAFGLLASGQAVKVILDPKA

SEQ ID NO: 5 - protein, 343 aa, synthetic construct; identical to SEQ ID NO: 4

SEQ ID NO: 6 - protein, 248 aa, synthetic construct
MIVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQ

SEQ ID NO: 7 - protein, 258 aa, synthetic construct
MTKTAVITGSTSGIGLAIARTLAKAGANIVLNGFGAPDEIRTVTDEVAGLSSGTVLHHPADMTKPSEIADMMAMVADRFGGADILVNNAGVQFVEKIEDFPVEQWDRIIAVNLSSSFHTIRGAIPPMKKKGWGRIINIASAHGLVASPFKSAYVAAKHGIMGLTKTVALEVAESGVTVNSICPGYVLTPLVEKQIPDQARTRGITEEQVINEVMLKGQPTKKFITVEQVASLALYLAGDDAAQITGTHVSMDGGWTAQ

SEQ ID NO: 8 - protein, 289 aa, synthetic construct
MEKVAFIGLGAMGYPMAGHLARRFPTLVWNRTFEKALRHQEEFGSEAVPLERVAEARVIFTCLPTTREVYEVAEALYPYLREGTYWVDATSGEPEASRRLAERLREKGVTYLDAPVSGGTSGAEAGTLTVMLGGPEEAVERVRPFLAYAKKVVHVGPVGAGHAVKAINNALLAVNLWAAGEGLLALVKQGVSAEKALEVINASSGRSNATENLIPQRVLTRAFPKTFALGLLVKDLGIAMGVLDGEKAPSPLLRLAREVYEMAKRELGPDADHVEALRLLERWGGVEIR

SEQ ID NO: 9 - protein, 348 aa, synthetic construct
MSEKMVAIMKTKPGYGAELVEVDVPKPGPGEVLIKVLATSICGTDLHIYEWNEWAQSRIKPPQIMGHEVAGEVVEIGPGVEGIEVGDYVSVETHIVCGKCYACRRGQYHVCQNTKIFGVDTDGVFAEYAVVPAQNIWKNPKSIPPEYATLQEPLGNAVDTVLAGPISGKSVLITGAGPLGLLGIAVAKASGAYPVIVSEPSDFRRELAKKVGADYVINPFEEDVVKEVMDITDGNGVDVFLEFSGAPKALEQGLQAVTPAGRVSLLGLYPGKVTIDFNNLIIFKALTIYGITGRHLWETWYTVSRLLQSGKLNLDPIITHKYKGFDKYEEAFELMRAGKTGKVVFMLK

SEQ ID NO: 10 - protein, 408 aa, synthetic construct
MHVPAAVRRSDPIARPATLRVQQPNAPSAADMAYIYYLTHIHLGYDALARLPEECARVGIRRPLIVTDKGVVAAGLVQCALDRLGAGHVPVFDDTPSNPTEAMVMAAAACYRHHDCDGLIAVGGGSAIDLAKGAAIAATHAGGLARYATVEGGSCRITEAVPPLIAVPTTAGSGSEVARGAALIVEDGRKLSFHSWHLMPRSAICDPGLTLGLPAMLTAGTGMDAIAHCIETFLAPAFNPPAEGIALDGLERAWTHIERATRDGRDRDARLQMMSASLQGAMAFQKGVGCAHALSHAIGGETRLHHGTLNAVLLPAVLRFNETAASVVQNRRYVRLRRAMGLPAEADIAQAVFEMNGRLGLPTGLKQLGVDASLFDRVIGAARTDYCHRTNPREATPADYRRLLSESL

SEQ ID NO: 11 - protein, 298 aa, synthetic construct
MTDIAFLGLGNMGGPMAANLLKAGHRVNVFDLQPKAVLGLVEQGAQGADSALQCCEGAEVVISMLPAGQHVESLYLGDDGLLARVAGKPLLIDCSTIAPETARKVAEAAAAKGLTLLDAPVSGGVGGARAGTLSFIVGGPAEGFARARPVLENMGRNIFHAGDHGAGQVAKICNNMLLGILMAGTAEALALGVKNGLDPAVLSEVMKQSSGGNWALNLYNPWPGVMPQAPASNGYAGGFQVRLMNKDLGLALANAQAVQASTPLGALARNLFSLHAQADAEHEGLDFSSIQKLYRGKD

SEQ ID NO: 12 - protein, 298 aa, synthetic construct
MKQIAFIGLGHMGAPMATNLLKAGYLLNVFDLVQSAVDGLVAAGASAARSARDAVQGADVVISMLPASQHVEGLYLDDDGLLAHIAPGTLVLECSTIAPTSARKIHAAARERGLAMLDAPVSGGTAGAAAGTLTFMVGGDAEALEKARPLFEAMGRNIFHAGPDGAGQVAKVCNNQLLAVLMIGTAEAMALGVANGLEAKVLAEIMRRSSGGNWALEVYNPWPGVMENAPASRDYSGGFMAQLMAKDLGLAQEAAQASASSTPMGSLALSLYRLLLKQGYAERDFSVVQKLFDPTQGQ

SEQ ID NO: 13 - protein, 314 aa, synthetic construct
MTEKVSVVGAGVIGVGWATLFASKGYSVSLYTEKKETLDKGIEKLRNYVQVMKNNSQITEDVNTVISRVSPTTNLDEAVRGANFVIEAVIEDYDAKKKIFGYLDSVLDKEVILASSTSGLLITEVQKAMSKHPERAVIAHPWNPPHLLPLVEIVPGEKTSMEVVERTKSLMEKLDRIVVVLKKEIPGFIGNRLAFALFREAVYLVDEGVATVEDIDKVMTAAIGLRWAFMGPFLTYHLGGGEGGLEYFFNRGFGYGANEWMHTLAKYDKFPYTGVTKAIQQMKEYSFIKGKTFQEISKWRDEKLLKVYKLVWEK

SEQ ID NO: 14 - protein, 260 aa, synthetic construct [sequence omitted]

SEQ ID NO: 15 - protein, 862 aa, synthetic construct
MKVTTVKELDEKLKVIKEAQKKFSCYSQEMVDEIFRNAAMAAIDARIELAKAAVLETGMGLVEDKVIKNHFAGEYIYNKYKDEKTCGIIERNEPYGITKIAEPIGVVAAIIPVTNPTSTTIFKSLISLKTRNGIFFSPHPRAKKSTILAAKTILDAAVKSGAPENIIGWIDEPSIELTQYLMQKADITLATGGPSLVKSAYSSGKPAIGVGPGNTPVIIDESAHIKMAVSSIILSKTYDNGVICASEQSVIVLKSIYNKVKDEFQERGAYIIKKNELDKVREVIFKDGSVNPKIVGQSAYTIAAMAGIKVPKTTRILIGEVTSLGEEEPFAHEKLSPVLAMYEADNFDDALKKAVTLINLGGLGHTSGIYADEIKARDKIDRFSSAMKTVRTFVNIPTSQGASGDLYNFRIPPSFTLGCGFWGGNSVSENVGPKHLLNIKTVAERRENMLWFRVPHKVYFKFGCLQFALKDLKDLKKKRAFIVTDSDPYNLNYVDSIIKILEHLDIDFKVFNKVGREADLKTIKKATEEMSSFMPDTIIALGGTPEMSSAKLMWVLYEHPEVKFEDLAIKFMDIRKRIYTFPKLGKKAMLVAITTSAGSGSEVTPFALVTDNNTGNKYMLADYEMTPNMAIVDAELMMKMPKGLTAYSGIDALVNSIEAYTSVYASEYTNGLALEAIRLIFKYLPEAYKNGRTNEKAREKMAHASTMAGMASANAFLGLCHSMAIKLSSEHNIPSGIANALLIEEVIKFNAVDNPVKQAPCPQYKYPNTIFRYARIADYIKLGGNTDEEKVDLLINKIHELKKALNIPTSIKDAGVLEENFYSSLDRISELALDDQCTGANPRFPLTSEIKEMYINCFKKQP

SEQ ID NO: 16 - protein, 365 aa, synthetic construct
MPAHSFRIAAIPGDGIGLEVLPEGIRVLEAAALKHGLALEFDTFEWASCDYYLQHGKMMPDDWAEQLKQYDAIYFGAVDWPDKVPDHISLWGSLLKFRREFDQYVNIRPVRLFPGVPCALANRKVGDIDFVVVRENTEGEYSSLGGIMFENTENEIVIQESIFTRRGVDRILKYAFDLAEKRERKHVTSATKSNGMAISMPYWDKRTEAMAAHYPHVSWDKQHIDILCARFVLQPERFDVVVVASNLFGDILSDLGPACAGTIGIAPSANLNPERNFPSLFEPVHGSAPDIFGKNIANPIAMIWSGALMLEFLGQGDERYQRAHDDMLNAIERVIADGSVTPDMGGTLSTQQVGAAISDTLARLD

SEQ ID NO: 17 - protein, 295 aa, synthetic construct
MRIAFIGLGNMGAPMARNLIKAGHQLNLFDLNKAVLAELAELGGQISPSPKDAAANSELVITMLPAAAHVRSVYLNEDGVLAGIRPGTPTVDCSTIDPQTARDVSKAAAAKGVDMGDAPVSGGTGGAAAGTLTFMVGASTELFASLKPVLEQMGRNIVHCGEVGTGQIAKICNNLLLGISMIGVSEAMALGNALGIDTKVLAGIINSSTGRCWSSDTYNPWPGIIETAPASRGYTGGFGAELMLKDLGLATEAARQAHQPVILGAVAQQLYQAMSLRGEGGKDFSAIVEGYRKKD

SEQ ID NO: 18 - protein, 324 aa, synthetic construct
MNPLLISSGEPAGIGPDLCLALAETDLPVVILGDLSLLEARASELNLSIKFLEYSPHQSFKKKAGYLTVWPVPCAAPVISGELNPQNAAYVMELLTLGASLCSKGEFSALVTAPVHKANINAAGITFTGHTEFFADFFEVETVVMMLACSQMKVALVTTHLPLRMVPDAISSLLIIKVIQQLHHSLKHDFGIQSPKINVAGLNPHAGESGYLGREEIEIITPALNTLKNQGIDVLGPLPADTMFITNHINHCDAYVAMYHDQGLPVLKYAGFNEAVNITLGLPIIRTSVDHGTALELAGKNKANPGSMLAAVKMAKDMALTRIK

SEQ ID NO: 19 - protein, 401 aa, synthetic construct
MESFDFFRRTRIIFGQSADNEVGQIIKYQGGTRVLLLHGEKAAIKYGIVERIGRTLDRSGLKYFSKGGIKSNPHIDKVYECIEFCLSNSINYILAVGGGSVIDTAKIVAAGVFFDGDIWDMFEKHREPYRSLPLGCVVTVPASGTECSNSSSLMREKDGRREKLIAYSNSFVPEFAILNPDLTLSLSPRVTASGCVDMINHVLEGYFSNSTGVLLSDKLCEAVLSSIIELLPQIYEDPNNIDARANLMLAATLSHNDICCMGRKSDNVITKLANQLVVENDCPFGDALAVLIPAWMEYVVQFNPLRIAQFSNRVFGIAINFEDPKITAYDGIKALRAFFKNVKLPCNFVELGIKTEAIADIVNALDLKEGKTLGSFVPLDAVACEAILSLAANYCEGRDIF

SEQ ID NO: 20 - protein, 363 aa, synthetic construct
MASSASKTNIGVFTNPQHDLWISEASPSLESVQKGEELKEGEVTVAVRSTGICGSDVHFWKHGCIGPMIVECDHVLGHESAGEVIAVHPSVKSIKVGDRVAIEPQVICNACEPCLTGRYNGCERVDFLSTPPVPGLLRRYVNHPAVWCHKIGNMSYENGAMLEPLSVALAGLQRAGVRLGDPVLICGAGPIGLITMLCAKAAGACPLVITDIDEGRLKFAKEICPEVVTHKVERLSAEESAKKIVESFGGIEPAVALECTGVESSIAAAIWAVKFGGKVFVIGVGKNEIQIPFMRASVREVDLQFQYRYCNTWPRAIRLVENGLVDLTRLVTHRFPLEDALKAFETASDPKTGAIKVQIQSLE

SEQ ID NO: 21 - protein, 267 aa, synthetic construct
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 22 - protein, 413 aa, synthetic construct
MTNSTDGRADGRLRVVVLGSTGSIGTQALQVIADNPDRFEVVGLAAGGAHLDTLLRQRAQTGVTNIAVADEHAAQRVGDIPYHGSDAATRLVEQTEADVVLNALVGALGLRPTLAALKTGARLALANKESLVAGGSLVLRAARPGQIVPVDSEHSALAQCLRGGTPDEVAKLVLTASGGPFRGWSAADLEHVTPEQAGAHPTWSMGPMNTLNSASLVNKGLEVIETHLLFGIPYDRIDVVVHPQSIIHSMVTFIDGSTIAQASPPDMKLPISLALGWPRRVSGAAAACDFHTASSWEFEPLDTDVFPAVELARQAGVAGGCMTAVYNAANEEAAAAFLAGRIGFPAIVGIIADVLHAADQWAVEPATVDDVLDAQRWARERAQRAVSGMASVAIASTAKPGAAGRHASTLERS

SEQ ID NO: 23 - protein, 348 aa, synthetic construct
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 24 - protein, 349 aa, synthetic construct
MRAARLHRYDTSLEGPEYLVLEEIPDPTIVEPDDVIVRIDGAGVCRTDLHIIEGQFRDHIPIRLPYTLGHENAGFVAAVGPGVQRFKPGDPVIVYPQVTDGTCLACRRGRDMYCENGEFPGLTRDGGFAEYLLTKERNLVRLPDGLSPRAVAPHADAGLTAYHAVKRALRWLDPGDTVALIGFGGLGHIAFQLLQVLAGVRTIVIDRAELALHLARELGATWTIQAGPESDVVSEVRQRTGGRGAAVVLDFVGEGLTPAQGLAMTRRGGAYFVIGYGGRLEVPTMELVNQEKAIIGNLVGTYTELAELMELVGEGRIKVAVTYYPLTQVNQALRDLRDGRVKGRAVVVP

SEQ ID NO: 25 - protein, 301 aa, synthetic construct
MEKSIKIGFIGLGAMGKPMAINLLKEGVTVYAFDLMEANVAAVVAQGAQACENNQKVAAASDIIFTSLPNAGIVETVMNGPGGVLSACKAGTVIVDMSSVSPSSTLKMAKVAAEKGIDYVDAPVSGGTKGAEAGTLTIMVGASEAVFEKIQPVLSVIGKDIYHVGDTGAGDAVKIVNNLLLGCNMASLAEALVLGVKCGLKPETMQEIIGKSSGRSYAMEAKMEKFIMSGDFAGGFAMDLQHKDLGLALEAGKEGNVPLPMTAMATQIFEGGRAMGLGREDMSAVIKVWEQMTGVSVSGGQ

SEQ ID NO: 26 - protein, 294 aa, synthetic construct
MKVGFIGLGIMGKPMSKNLLKAGYSLVVADRNPEAIADVIAAGAETASTAKAIAEQCDVIITMLPNSPHVKEVALGENGIIEGAKPGTVLIDMSSIAPLASREISEALKAKGIDMLDAPVSGGEPKAIDGTLSVMVGGDKAIFDKYYDLMKAMAGSVVHTGEIGAGNVTKLANQVIVALNIAAMSEALTLATKAGVNPDLVYQAIRGGLAGSTVLDAKAPMVMDRNFKPGFRIDLHIKDLANALDTSHGVGAQLPLTAAVMEMMQALRADGLGTADHSALACYYEKLAKVEVTR

SEQ ID NO: 27 - protein, 352 aa, synthetic construct
MKGFAMLSIGKVGWIEKEKPAPGPFDAIVRPLAVAPCTSDIHTVFEGAIGERHNMILGHEAVGEVVEVGSEVKDFKPGDRVVVPAITPDWWTSEVQRGYHQHSGGMLAGWKFSNVKDGVFGEFFHVNDADMNLAHLPKEIPLEAAVMIPDMMTTGFHGAELADIELGATVAVLGIGPVGLMAVAGAKLRGAGRIIAVGSRPVCVDAAKYYGATDIVNYKDGPIESQIMNLTEGKGVDAAIIAGGNADIMATAVKIVKPGGTIANVNYFGEGEVLPVPRLEWGCGMAHKTIKGGLCPGGRLRMERLIDLVFYKPVDPSKLVTHVFQGFDNIEKAFMLMKDKPKDLIKPVVILA

SEQ ID NO: 28 - protein, 296 aa, synthetic construct
MKKIGFIGLGNMGLPMSKNLVKSGYTVYGVDLNKEAEASFEKEGGIIGLSISKLAETCDVVFTSLPSPRAVEAVYFGAEGLFENGHSNVVFIDTSTVSPQLNKQLEEAAKEKKVDFLAAPVSGGVIGAENRTLTFMVGGSKDVYEKTESIMGVLGANIFHVSEQIDSGTTVKLINNLLIGFYTAGVSEALTLAKKNNMDLDKMFDILNVSYGQSRIYERNYKSFIAPENYEPGFTVNLLKKDLGFAVDLAKESELHLPVSEMLLNVYDEASQAGYGENDMAALYKKVSEQLISNQK

SEQ ID NO: 29 - protein, 347 aa, synthetic construct
MIETKGYATHGAKEALKPFSFQRRDVGADDVLIDIHYCGICHSDIHQARNEWGQSTYPMVPGHEIVGKVSAVGANVRGFKVGDLAGVGCFVDSCGVCPSCREGEEQYCDQSPVWTYNAVEKDGKTPTYGGYSQKIVVKEKYCLRVSPNLPLSHVAPLLCAGITTYSPLRHFGVKSGDKVGVVGLGGLGHMGVKLAASMGARVTVFSTSDKKAADSKRLGAESFVVTRDPGNMAALANSFDFILDTVSAPHDINAYLNLLRRDGQLVFVGVPEKPIEVQPFAMIGKRRSMAASLIGGIRETQEMLDYCASKGITSDVEVIGVDRIMEAYDRTVKGDVRYRFVIDMKTL

SEQ ID NO: 30 - protein, 381 aa, synthetic construct
MDKFTFSYPTKVYFGEESAAQAFGAELGKVGKTVMLAYGGGSVKKNGVYDEIKSLLEQAGKTIIDFSGIMPNPTYAKVQEGAAIVRKQHVDFILALGGGSVIDCCKVISAQAVLDEDIWEMEYSKGQFPTAGIPIGAVVTASGTGAEMNAGAVITYEEKKWKGPVFGTVASFAVLDPAYTMSVPPMQVFSGAFDTLSHAMETYFGSSDQNNVSDDLALAVMRNTVVNMRRLLKNINDRQARSNLMWDSAMAENGILKCGRLTDFQAHQMEHQLGAYTDCNHGQGLAVIHPAYYRHIVKDAPEKFARLAKVVFDVDTAAGGVQALESFIKECGLPTKMGQLKSTVEITPEVLRQVADTCNIIKVGPRELSRDEIYDILMECL

SEQ ID NO: 31 - protein, 202 aa, synthetic construct
MTATDQAPALEIEQHVQDLLFREARTANTFSDEPVTDEQVRAIYDLVKWGPTSMNQQPLRVVLVRSDEAKQRLLPLMGEGNRDKTGKAPLVALLGTDTEFHEKLPRLFPHFPGARDVWTDDDFRTRHALLNASLQIGYFIVGVRAAGLAAGPMTGFDADAVTKEFFPEGQQALVAVNLGKPGPDAWFERLPRLDYDEVVTTV

SEQ ID NO: 32 - protein, 388 aa, synthetic construct
MDAFTFTTVPQLVFAPGAARELAVHCRALGVARPLLVTDPGLVGLGLVPPLEALLSGDGLVVTRFAEVCEDPPEAVVLACVEQARAAGVDGVIAVGGGSSMDTAKVVAVLLGGDQGLAELYGVDQVRGGRLPLLLVPTTAGTGSEATPVAVITTGETTKAGVSSSVLLPDRAILDPALTLGLPPRVTAMTGIDAMVHAIEAYTSRHRKNPVSDHLARSALKLLAGNLRTAVLDGDNLEARSAMLLGAFQAGQAFANAPVAAVHALAYPLGGHYHIPHGLSNSLVLPSVLAFNAPVAAPLYAELAPLVLGRPVEGSPEAVTEILVAGLRQLIADVSLPATLAEAGVSADDLPMLADDAMLQQRLLVNNPREVAREDALAIYRAAHGAAA

SEQ ID NO: 33 - protein, 336 aa, synthetic construct
MSIPSSQYGFVFNKQSGLNLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 34 - protein, 389 aa, synthetic construct
MTLNTYSFELATKVVFGPGTIEEAGREAKKLGMTKAMIIADQGIIHAGLTAPVEAALLEEDIHFVIYDKIVPNPRDFHCLEGAEFGREHEIDGMIAIGGGSSIDTAKAIGTLMTNGKDIRTWAGLNLLEREIPPLIAIPTTAGTGSEVTFDAVITDTKTHEKLNILDLKIAPKVALVDPQTLLKLPSRAMAACGIDALVHAVEAYTCKAACPHTDAYALYAIDIIQKNLRSAVESPDTDNCTGMALGSTMAGIAFGYSDVAAVHCIAEALGGRYDTPHGVANAIMLPVVTEFNIPSNLEKYANMAKYLAVDTSNLTKEEAAYSCVGALQKLCEDVKIPKMREVEGISPVDFPELAEASFRNISTLDNPREITVDDYLYLLNKAYFELSD

SEQ ID NO: 35 - protein, 292 aa, synthetic construct
MEHKTLSIGFIGIGVMGKSMVYHLMQDGHKVYVYNRTKAKTDSLVQDGANWCNTPKELVKQVDIVMTMVGYPHDVEEVYFGIEGIIEHAKEGTIAIDFTTSTPTLAKRINEVAKRKNIYTLDAPVSGGDVGAKEAKLAIMVGGEKEIYDRCLPLLEKLGTNIQLQGPAGSGQHTKMCNQIAIASNMIGVCEAVAYAKKAGLNPDKVLESISTGAAGSWSLSNLAPRMLKGDFEPGFYVKHFMKDMKIALEEAERLQLPVPGLSLAKELYEELIKDGEENSGTQVLYKKYIRG

SEQ ID NO: 36 - protein, 549 aa, synthetic construct
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANI

SEQ ID NO: 37 - protein, 354 aa, synthetic construct
MEKRIVCLAGDGVGPEVMESAKEVLHMVERLYGHHFHLQDEYFGGVAIDLTGQPLPQRTLAACLASDAVLLGAVGGPRWDSAKERPEKGLLALRKGLGVFANVRPVTVESATAHLSPLKNADEIDFVVVRELTGGIYFSYPKERTEEVATDTLTYHRHEIERIVSYAFQLANKRKKKVTSIDKANVLESSKLWRTVTEEVALRYPNVEVEHILVDAAAMELIRNPGRFDVIVTENLFGDILSDEASVLAGSLGMLPSASHAEKGPSLYEPIHGSAPDIAGKNKANPIAMMRSVAMMLGQSFGLTREGYAIEEAVSAVLKSGKCTADIGGTETTTSFTKAVIQEMEEQALVGRGR

SEQ ID NO: 38 - protein, 350 aa, synthetic construct
MSIPTMQWAQVAEKVGGPLVYKQIPVPKPGPDQILVKIRYSGVCHTDLHAMMGHWPIPVKMPLVGGHEGAGIVVAKGELVHEFEIGDQAGIKWLNGSCGECEFCRQSDDPLCARAQLSGYTVDGTFQQYALGKASHASKIPAGVPLDAAAPVLCAGITVYKGLKEAGVRPGQTVAIVGAGGGLGSLAQQYAKAMGIRVVAVDGGDEKRAMCESLGTETYVDFTKSKDLVADVKAATPDGLGAHAVILLAVSEKPFQQATEYVRSRGTIVAIGLPPDAYLKAPVINTVVRMITIKGSYVGNRQDGVEALDFFARGLIKAPFKTAPLKDLPKIYELMEQGRIAGRYVLEMPE

SEQ ID NO: 39 - protein, 309 aa, synthetic construct
MRKKISIIGAGFVGSTTAHWLAAKELGDIVLLDFVEGVPQGKALDLYEASPIEGFDVRVTGTNNYADTANSDVIVVTSGAPRKPGMSREDLIKVNADITRACISQAAPLSPNAVIIMVNNPLDAMTYLAAEVSGFPKERVIGQAGVLDAARYRTFIAMEAGVSVEDVQAMLMGGHGDEMVPLPRFSTISGIPVSEFIAPDRLAQIVERTRKGGGEIVNLLKTGSAYYAPAAATAQMVEAVLKDKKRVMPVAAYLTGQYGLNDIYFGVPVILGAGGVEKILELPLNEEEMALLNASAKAVRATLDTLKSL

SEQ ID NO: 40 - protein, 321 aa, synthetic construct
MSFVTEIKTFAALGSGVIGSGWIARALAHGLDVVAWDPAPGAEAALRARVANAWPALRKQGLAPGAAQERLRFVASIEECVGDADFIQESAPERLDLKLDLHARISAAARPDVLIGSSTSGLLPSEFYAEASHPERCLVGHPFNPVYLLPLVEVVGGERTAAEAVRAAMRVYESLGMRPLHVRKEVPGFIADRLLEALWREALHLVNDGVATTGEIDDAIRFGAGLRWSFMGTFLTYTLAGGNAGMRHFMAQFGPALQLPWTYLPAPELTEALIDRVVEGTAEQQGARSIAELERYRDDCLLAVLGAIRETKARHGFAFAE

SEQ ID NO: 41 - protein, 290 aa, synthetic construct
MSSPKIGFIGYGAMAQRMGANLRKAGYPVVAYAPSGGKDETEMLPSPRAIAEAAEIIIFCVPNDAAENESLHGENGALAALTPGKLVLDTSTVSPDQADAFASLAVEHGFSLLDAPMSGSTPEAETGDLVMLVGGDEAVVKRAQPVLDVIGKLTIHAGPAGSAARLKLVVNGVMGATLNVIAEGVSYGLAAGLDRDVVFDTLQQVAVVSPHHKRKLKMGQNREFPSQFPTRLMSKDMGLLLDAGRKVGAFMPGMAVADQALALSNRLHANEDYSALIGAMEHSVANLPHK

SEQ ID NO: 42 - protein, 434 aa, synthetic construct
MSFNTIIDWNSCTAEQQRQLLMRPAISASESITRTVNDILDNVKARGDEALREYSAKFDKTTVTALKVSAEEIAAASERLSDELKQAMAVAVKNIETFHTAQKLPPVDVETQPGVRCQQVTRPVASVGLYIPGGSAPLFSTVLMLATPASIAGCKKVVLCSPPPIADEILYAAQLCGVQDVFNVGGAQAIAALAFGTESVPKVDKIFGPGNAFVTEAKRQVSQRLDGAAIDMPAGPSEVLVIADSGATPDFVASDLLSQAEHGPDSQVILLTPAADMARRVAEAVERQLAELPRAETARQALNASRLIVTKDLAQCVEISNQYGPEHLIIQTRNARELVDSITSAGSVFLGDWSPESAGDYASGTNHVLPTYGYTATCSSLGLADFQKRMTVQELSKEGFSALASTIETLAAAERLTAHKNAVTLRVNALKEQA

SEQ ID NO: 43 - protein, 383 aa, synthetic construct
MDFEFFMPAQLIFGRGKAKCIGSYVKSIANRALIVTGKSSAKKAGYLDLVTKSLEKEGIDWALFDEVEPNPLTTTIDRGISLARKSKAEVIIGLGGGSAMDSAKCIAFGVKKEDSIANYFGTQPAGEVLPIVLITTTAGTGSEANNYAVMTNPETHVKKSLSSPDMFAKISIIDPELMLTVPKKVTASTGIDVFFHSMESYLSKRCQPISRPLSLEAMRLVVENLKGAYEHGDNIEYRERMAWANTIAGIAINLAGNCGIHGLGHPLSAYYNIAHGETLAAVSLAFLRFSIPEASDKLATIAEIFGVETKGLSKDEAAEKAIEALETFMESLALPTKISVFGVKEEDIDKLAQNAYENSIGNFKASPREITLDDARKIYRDSL

SEQ ID NO: 44 - protein, 345 aa, synthetic construct
MKVAVLPGDGIGPEVTEAALKVLRALDEAEGLGLAYEVFPFGGAAIDAFGEPFPEPTRKGVEEAEAVLLGSVGGPKWDGLPRKIRPETGLLSLRKSQDLFANLRPAKVFPGLERLSPLKEEIARGVDVLIVRELTGGIYFGEPRGMSEAEAWNTERYSKPEVERVARVAFEAARKRRKHVVSVDKANVLEVGEFWRKTVEEVGRGYPDVALEHQYVDAMAMHLVRSPARFDVVVTGNIFGDILSDLASVLPGSLGLLPSASLGRGTPVFEPVHGSAPDIAGKGIANPTAAILSAAMMLEHAFGLVELARKVEDAVAKALLETPPPDLGGSAGTEAFTATVLRHLA

SEQ ID NO: 45 - protein, 395 aa, synthetic construct
MRAVRLHAKWDPRPEFKLGPKDIEGKLTWLGSKVWRYPEVRVEEVPEPRIEKPTEIIIKVKACGICGSDVHMAQTDEEGYILYPGLTGFPVTLGHEFSGVVVEAGPEAINRRTNKRFEIGEPVCAEEMLWCGHCRPCAEGFPNHCENLNELGFNVDGAFAEYVKVDAKYAWSLRELEGVYEGDRLFLAGSLVEPTSVAYNAVIVRGGGIRPGDNVVILGGGPIGLAAVAILKHAGASKVILSEPSEVRRNLAKELGADHVIDPTKENFVEAVLDYTNGLGAKLFLEATGVPQLVWPQIEEVIWRARGINATVAIVARADAKIPLTGEVFQVRRAQIVGSQGHSGHGTFPRVISLMASGMDMTKIISKTVSMEEIPEYIKRLQTDKSLVKVTMLNE

SEQ ID NO: 46 - protein, 381 aa, synthetic construct
MYPFSFHNPTRIEFGVDKEQFMGAYMREYGAKRALIVYGSERVKQSGLFARVANGLREQGIEFIECGGIKSNPVLSKVREAVQMAKDFKADSVLSIGGGSCLDSAKAIAAGACYAGDVWDFFKGTPIPQALMIFDVITLAATGSEMNWGAVITNEETRQKDSIHDRLLFPKVSVINPQLQATVSREYLVYSAADIIAHSIEAYFTAENRPQIIDGLVENNIKSVIQTTEKLLADPNDLDARGEFAWAATLALNGLTHLGIGKYQFPNHMLEHSMSAICDVPHGAGLSVLMPAWMHWYQAHNPQRFIRFAREVFALDSAEQGIQALRAWFDKIGTPTRLSQLNITDAQLEEIIENAAQTAKQRNLDVLYSKAAISDIFALTR

SEQ ID NO: 47 - protein, 323 aa, synthetic construct
MVGTTTLNTGASLELVGYGTWQAAPGEVGQGVKVAIETGYRHLDLAKVYSNQPEVGAAIKEAGVKREDLFITSKLWNNSHRPEQVEPALDDTLKELGLEYLDLYLIHWPVAFPPEGDITQNLFPKANDKEVKLDLEVSLVDTWKAMVKLLDTGKVKAIGVSNFDAKMVDAIIEATGVTPSVNQIERHPLLLQPELIAHHKAKNIHITAYSPLGNNTVGAPLLVQHPEIKRIAEKNGCTPAQVLIAWAIVGGHSVIPKSVTPSRIGENFKQVSLSQEDVDAVSKLGEGSGRRRYNIPCTYSPKWDINVFGEEDEKSCKNAVKIK

SEQ ID NO: 48 - protein, 318 aa, synthetic construct
MNNRRKIVVIGASNVGSAVANKIADFQLATEVVLIDLNEDKAWGEAKDSSHATSCIYSTNIKFHLGDYEDCKDANIIVITAGPSIRPGETPDRLKLAGTNAKIMSSVMGEIVKRTKEAMIIMITNPLDVATYVVSTQFDYPRNLILGTGTMLETYRFRRILADKYQVDPKNINGYVLGEHGNAAFVAWSTTGCAGFPIDDLDEYFHRTEKLSHEAVEQELVQVAYDVINKKGFTNTGIAMAACRFIKSVLYDEHTILPCSAVLEGEYGIKDVALSIPRMVCADGIMRSFEVHLTDDELEKMHKAAQSVRSALDGAGIK

SEQ ID NO: 49 - protein, 386 aa, synthetic construct
MEAFTFSYPTKNYFGQGAAHKALAEELPKYGKHVLLAYGGGSVKRNGIYDELLAILKEQGKEVTEFAGIMSNPTYRKVQEGARLVREHKIDLILAVGGGSVSDCCKAVSAQAALEEDLYDYEYAKHKVPTAGVPMGVIVTASGTGSEQNAGAVITYEEKNWKGPLWGTAASFAVLDPYYTKTVPMRQVISGAFDTLSHCLETYLGTPREPNLSDVMNESVMRSVIENIRKVKADPEDMQARSELMWASAMAENGVLKLGKVTDFQCHMIEHQLGAYTDCNHGCGLAVIQPELYRHLAPEAPAQFARLAREVFGVAEQESELATALAGVEKFADFIEEIGLPTTLKAMGIEDKAMLHAVAVTSIRTPGCAKKLTDEEIEEILLHVLG

SEQ ID NO: 50 - protein, 390 aa, synthetic construct
MNNFIFYSPTEFVFGRDTEAQTGALVQKYGARKVMIVYGGGSVIRSGLLARVEKSLQEVGIPYCMLGGVQPNPIDTKVYEGIDLCRKESVDMMLAVGGGSVIDTAKAIAAGVPYDGDFWDFYIGKAVVAEALKVAVVLTIPAAGSEGSGNTVITKVDGLQKLSLRAPSVLRPVFAVMNPELTYTLPPFQTACGVADMMAHIMERYFTNTKEVEIGDRLCEGTLLAIIKEATTVMKDPENYGARANLMWCGTIAHNGTCGVGCEEDWASHFLEHEISAIYNVTHGAGLSVIFPAWMTWMTEHNVDKIAQYAIRVWGVADSADKKAVALEGISRLKTFFTSIGLPVTFKELGIENPDIDRLADSLHRNKGELVGNYVKLTKKDSKEIYRLAL

SEQ ID NO: 51 - protein, 392 aa, synthetic construct
MKFNYNMPTKILFGPGKLNSLKKEKLPGKKALIVISAGNSMKSNGYLDRVIEILNSKGIKAVIFDQILPNPVKAHVMEGAKIAVREECDFVIGLGGGSSIDSAKSIAVMAKNPGDYWDYISGGTGKGKPVENGALPVVAITTTAGTGTEADPWTVITKEDTKEKIGFGNADTFPVLSVVDPELMLTVPKKLTAYQGFDAFFHATEGYIAKIANPFSDLYALKSIELIARYLPRAVKDGNDLEARTQVALANTLSGFVESTSSCTSEHSMEHALSAYYPDLPHGAGLIMLSEAYYTFFASKVPERFISMAEAMGVNVDLLPENERPMAFVQALVKLQQDCEVDGLKMSDYGIKREEITLLADHARETMGGLFTVDPYSLSLEETTEIMKNAYK

SEQ ID NO: 52 - protein, 381 aa, synthetic construct
MKDFVFSYPTNVYFGEGAAKKALKSELCSAGSTVMVAYGRGSVKENGIYDEITAILKEMGKRVVDFSGIMSNPTYEKVQEGAALAKKEKADYILAVGGGSVIDCCKIIAAQAKTEEDIWEMEFVQGRFPKDTIPLGAVVTASGTGAEMNGGAVITNEELQIKTGVMACAPRFAVLDPSYTLSVPAMQVISGAFDTLSHAMETYLGQSDEDNVSDDVALAVMRNTVVNMRRLLADQNDLQARGNLMWDSAMAENGILKVGRLTDFQTHQMEHQLGAFTDCNHGQGLAVIHPAYYRHIVRDAEEKFTRFAREVFAVQTAEEGIEALSSFIRECGLPTKLRELKSRVEITPEVLRKVADTCNIIKCNPRELSRDEIYDILLECL

SEQ ID NO: 53 - protein, 390 aa, synthetic construct
MENFIYHNPTKLIFGKDQLQALSEELPKYGKNILLVYGGGSIKKNGLYDQVLTLLKELNVNIHELAGVEPNPRLSTVRKGIEICRNENIDFMLAVGGGSVIDCTKAIAAGAEYKGDVWDIINKKVTATKALPFGTVLTLAATGSEMNPDSVITNWETNEKYVWGSAVTHPVFSILDPVNTMSVPRDQTIYGMVDMMSHVFEQYFHNVKNTPLQDRMCYSVLQTVIETAPKLIEDLQSYEHRETILYSGTIALNGTLQMGYFGDWASHTIEHAVSAVYDIPHAGGLAILFPNWMRHVVDQDVTRMKRVMTSMFDIETAGKSDKEIALEGIDKLSAFWSSLGAPSRLADYDIGEDRLDEIAGIAAREMEYGGFGNFKKLNKDDILAILKASL

SEQ ID NO: 54 - protein, 393 aa, synthetic construct
MKKEGLIMNDFRFYNPVRIHFGQSSLNKLPKELAAVGKTILLAYGGGSIKRTGLYNRVMAILEDCGKEVVELSGIMPNPRTEKVYEGIELCKKHNVDFILAVGGGSTIDCSKAIAVGACTDRDFWEAFYLNNEAAEAAIPLGVVLTIPATGSEMDRSAVITHWETGVKTDYNSDLTFPKFSILDPTLTYTLPKNQMINGIVDTLSHIWELYFSEPDTDSVTDNLAEALMRSVIAATRVAIKEPEDYIARSNIMWASTVALCGMIGNGKNSDWASHNIEHAISALFDVAHGAGLAVVHPTYMAYFCQNAPERYARYAVNVWGVDPTGKTALEVAREGVQCTRDFFNEIGAPATLTDLGVPESAIDDLVAHTDMGAWAYKKMTQDDVRAILRMAL

SEQ ID NO: 55 - protein, 402 aa, synthetic construct
MNDFTFLSPTRLVVGRDAEKETGKWIKEYGGTTVLVHHDSGFVKQNGFVDKVIDMLKADGLKVVELGGVVPNPHLSKVYEGIELCKKEGVDFLLAIGGGSVIDSTKAIAMGLPYEGDVWDFFIEEDGVPHDVPTKSTPLGVILTIAATGSEASNSCVITKADENLKRFCDNDINRARFAIENPELTMTLPPFQTACGIIDIMSHSFERYFTPEKENDILTDYLCEAIFHTCMDCGRILMKDPENYEARASIMVASTLSHNGLTGMGRTGDWASHFIEHELSGEYQSVTHGAGLAVITPAWMKYVYKDNMDKFIKWATRVMGVSMDYGDPERTVLEAIDRLELFFLSLGVPTKLRDVPGVEDLDEEVMERMAKRVRVVHEDGSIGWVRRLNTEDIVEIFKLAM

SEQ ID NO: 56 - protein, 311 aa, synthetic construct
MKVAVLGAAGGIGQALALLLKNRLPAGSELSLYDIAPVTPGVAVDLSHIPTDVAIKGFCGEDPTPALVGADVVLISAGVARKPGMDRSDLFNINAGIVKNLVEKCAATCPKALIGIITNPVNTTVAIAAEVLKKAGVYDKRRLFGVTTLDVIRAETFVAAAKGLNIDKVRVNVIGGHSGVTILPLLSQIEGASFTAEEVAAMTKRIQNAGTEVVEAKAGGGSATLSMGQAACRFGLSLIKGLQGEANVVECAYVEGNGEHATFFAQPVLLGKNGVETVLDYGKLSAFEQEAMEGMLATLKADIQLGVDFVK

SEQ ID NO: 57 - protein, 387 aa, synthetic construct
MKNFDYNIPTRVHFGKGQISKLRGEVKNFGHNVLLVYGGNSIKRNGIYDQVMEQLNDSDINVFELSGIAPNPRIDSARDGVKLCKECKIDVILAVGGGSVIDCSKLIAAGAFYDGDPWDLVLKSSLIKEALPIISVLTLSATGSEMDGFAVISNMETNDKLGMGGEILKPRVSILDPEYTYTVPANQTAAGTADIMSHIIETYFHDVEGAYLQDRFAESLLKTCIEFGPIAVKEPENYDARANLMWAGSHAINGMISYGTDVGWSVHPMEHELSAYYDITHGVGLAILTPRWMKHILSDKTVGKFVDYGVNVWGIDSSLDKFEIANMAIEKTYDFFASIGIPMNLREVGIDEENLEVMAEFASKRGAKYGYVSLSKEDVLEIYKMSL

SEQ ID NO: 58 - protein, 394 aa, synthetic construct
MLNFEYHTPTKVVFGKDTESKAGSLIKEQGCRKVLVHYGSGSVIRSGLLDRVYASLDAEGIPYASLGGVVPNPRLTKVREGIALCQREGVDFILAVGGGSVIDSSKAIGYGVANEGDVWDFYARKRTASACLPIGAILTIAAAGSEMSNSSVITNEDGLKKRGYKSEYCRCRFAILNPELTYTLPDYQTQSGCVDVLMHTMERYFNQSTNMEMTDGISETLLRTVMKNSLILKKDPQNYDARAEVMWASSLSHNGLTGCGTDGGDWASHQLEHELGGMFDVAHGAGLAAIWGTWARYVCKDRLDRFAKFAENVLLIPHSEDLEQMALLGIQAMEDYFRSIGMPTSLHKLGIAPSEEQILELADKCSFGNTRTIGKVRELNREHIADIYRLARGI

SEQ ID NO: 59 - protein, 546 aa, synthetic construct
MKRRVGQYLMDAVHAAGVDKIFGVPGDFNLAFLDDIVRHDGVEWIGTTNELNGSYAADGYARIHGLGVLVTTFGVGELSAVNGIAGSYAERVPVIALTGAPTRAVENAGKYVHHSLGEGKFDSYRKMFEPITTAQAYITPENATIEIPRVINAALRERRPVHIHLPIDVAMEEIEIGNPYEVALTPKQDLSRYVDMVADKLNHAKQPVIIAGHEINSFHLHDELEHFVNKTNIPVVQLSLGKGAFNEENEHYIGIFDGKIADEEIKDYVNNSDVILNIGAKLTDSATAGFAYEFDVDEVIMLNHRHFKLGNIVEEDVTLPNLLADLNNLDYHYDGTFPTFKKAEEGNYELTNDALTQETYFKMMQEFIGGDDILIAEQGTSFFGSYELALPQGMSFIGQPLWGSIGYTLPSTLGTQMADASRRNILLIGDGSLQLTVQELSTMIRHNIKPIIFVINNDGYTVERLIHGMEEPYNDIRMWDYKALPKVFGGDNVAVHDVKTSNDLKRVFDEINDAPDQMHFTEVRMNWDDAPEKLRDISKAFAEQNK

SEQ ID NO: 60 - protein, 549 aa, synthetic construct
MNKTVIEYVLDRLYQIGIHDIFGVAGDYAFPIEDAVCESENMRWIGNCNELNASYAADGYARVKGAAALSTTFGVGELSALNGIAGAYAEHLPVFHLVGMPASGVQKKHRLVHHTLGNGDFDVFYQMSQHLSCAHAILTPENCIAETERLITTALQESRPVYLGFPSDYAVMPIKADETPETITINKSHSKSLSAAVTAIVEKLTSSTKACIIPGILTARFGLTTDVEAIIQKTGLPYATMFMDKGVLSESNSQYMGIYNGKLMNPDVREFVESCDCVMGIGAVLTDFNTGSFTATIAPESCINILSDHVKVGSAIYPNVLMKDVLDKLKELAPSLNHAGLKAQGLGSPQQGENGQITASYLYPRLEKMFRKDDIIIAETGTASMGLGFALLPENAQFHNQTLWGSIGWATPAAFGAAIAEPQKRVILVTGEGSHQLTAQEISQFARFGLKPIIFVLNNDGYLIERLLCNNPEAYYNDLPQWNYAQLPAALGCNDWYCQRVTNCNELDNAIKQAELSNHAAYIEVVMDRYAASELAEKLGKSVASLYSF

SEQ ID NO: 61 - protein, 563 aa, synthetic construct
MSEITLGRYLFERLKQVEVQTIFGLPGDFNLSLLDNIYEVPGMRWAGNANELNAAYAADGYARLKGMSCIITTFGVGELSALNGIAGSYAEHVGVLHVVGVPSVSSQAKQLLLHHTLGNGDFTVFHRMSSNISETTAMITDINTAPAEIDRCIRTTYVSQRPVYLGLPANLVDLTVPASLLDTPIDLSLKPNDPEAEEEVIENVLQLIKEAKNPVILADACCSRHDAKAETKKLIDLTQFPAFVTPMGKGSIDEKHPRFGGVYVGTLSSPAVKEAVESADLVLSVGALLSDFNTGSFSYSYKTKNIVEFHSDYTKIRSATFPGVQMKFALQKLLTKVADAAKGYKPVPVPSEPEHNEAVADSTPLKQEWVWTQVGEFLREGDVVITETGTSAFGINQTHFPNNTYGISQVLWGSIGFTTGATLGAAFAAEEIDPKKRVILFIGDGSLQLTVQEISTMIRWGLKPYLFVLNNDGYTIERLIHGETAQYNCIQNWQHLELLPTFGAKDYEAVRVSTTGEWNKLTTDEKFQDNTRIRLIEVMLPTMDAPSNLVKQAQLTAATNAKN

SEQ ID NO: 62 - protein, 568 aa, synthetic construct
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAEGGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 63 - protein, 547 aa, synthetic construct
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 64 - protein, 547 aa, synthetic construct
MAKAADLVVQCLESEGVEYVFGIPGEENLDLLESLRKSRVKLVLTRHEQSAGFMAATYGRLTGKTGVSLSTLGPGATNLVTASAYAYLGGMPMMMITGQKPIKKSKQGRFQIIDVCGMMAPITKYTHQFASADNIPARMREAFRLAEEEKPGAVHLELPEDIAAEQTDALPIPQSLHRRPLAEHKAIEAAVEKLHSARNPILVIGAGANRKMTAKVLKQLIDETRIPFITTQMGKGVVDERHPCFLGNAALSSGDFVHRAIEAADLIINIGHDVIEKPPFFMVRGGTEVIHISFRAAEVDAVYFPQVEVIGDIANAVWQINEALADTSHWDFTCLMAIREANEAQISEGADDARFPVYPQRLVADIRRVLPSEGIVALDNGIYKIWFARNYKAHKPNTVLLDNALATMGAGLPSAMAAHLVYPDRPVISVCGDGGFMMNSQELETAVRLGMHITVVILRDDGYGMIRWKQANMGFIDFGLDYGNPDFVKYAESYGANGYRVESADSLIPLLDHCVSNPGVHVIDCPVDYSDNDRILNTDLRERSAAV

SEQ ID NO: 65 - protein, 554 aa, synthetic construct
MTTYTVADYLLDRLAEAGVRHLFGVPGDFTLDFLDHVERHPVVEWVGCANELGAGYAADGYGRLHGLGALSTTFGVGELSAISAVAGSYAEHVPVVHVVGAPTTAVQAAGRASHHSLGDGDFGHFARMTAEVTAAQEVLSATDAPAQIDRLLAEVRHRRLPGYLLIPADVGGAPVEPPSSPLPDPDVVTDPVVLARFRDAVAARLEASDTVAVLADILVSRLGAEDALHGLLGHGLAHASLLWGRRVVDESASGYLGSYLGAASAPAVRTGIEGADLLVLAGVQFTDLTTGFFSQRIDAARTVEIRGEHARVGDDDYCPLAMRDALDAVSAEVAVRSDRLRAHDSPSTPTPPRTPDADGPLSQRALWHEVAAFLREGDTVLADQGTSFYGMAGERLPHGVVFGGQPLWAAIGFTLPAILGAALARPERRPVLLIGDGAAQLTAAELGTLVRHDVPAVIVVVDNAGYTVERVIHGLHEEYNDIAAWDWTTLLAAMDPRGATTGVRVDTVEDLRRALTDARTSSSLTLIQAVVPQDDVPPVLADLAAAAAAANHRA

SEQ ID NO: 66 - protein, 633 aa, synthetic construct
MYRLKNLQAVPWKAPTRAIRGIALDGPDRDRMFNAYLHSSRPSFGPATDVFVQCLEAEKIDWIFGIPGEENLDLLHSLAKAHRREGGETMKLVVTRHEQAAGFMAATVGRLTGHPAACLSTLGPGATNFTTALAYSKLGGFPLICITGQKPIRHSKQGAFQILDICGHFRDVVKSTKSIEDPDLIPSTIRNAVLQAREEKPGPVHIELAEDIAALEVSKEGGWVFPAPTRTPIARGRRPLVEPKCIEHAVTLIHAAKRPLICIAAGANRKNTHHAMEVLMRKTGLMVVCTQMGKGVVYEDNLGYIGCTALSDKDLVHVAINTADLIVNVGHDISEKPPFIMNHNKPPLVIHANFTRPLVDPVYFPHLVLVGDINDCLWQLATRIRPQPHWDFNPFQMVRREIERTVWNSPYAQSDAFPLTVQRLVSDVRKAMPVDGIVSLDNGMYKIWFARQYKTMMGNTLLLDNALATMGAGLPSCIAAKLVYPERVCIAVCGDGGFMMNSQEIETALRLNLHIVVIVLNNNSYGMIAWKATAMGMDDFGLNYGNPDFAQYARAYGAIGHNVKSTAEFLPTLEKAIKEHGVHIIDLPISYETSDKALFEDLPKEVEELKKAVAKAISEEKKFDWDAVTAAQS

SEQ ID NO: 67 - protein, 548 aa, synthetic construct
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 68 - protein, 609 aa, synthetic construct
MNSSYTQRYALPKCIAISDYLFHRLNQLNIHTIFGLSGEFSMPLLDKLYNIPNLRWAGNSNELNAAYAADGYSRLKGLGCLITTFGVGELSAINGVAGSYAEHVGILHIVGMPPTSAQTKQLLLHHTLGNGDFTVFHRIASDVACYTTLIIDSELCADEVDKCIKKAWIEQRPVYMGMPVNQVNLPIESARLNTPLDLQLHKNDPDVEKEVISRILSFIYKSQNPAIIVDACTSRQNLIEETKELCNRLKFPVFVTPMGKGTVNETDPQFGGVFTGSISAPEVREVVDFADFIIVIGCMLSEFSTSTFHFQYKTKNCALLYSTSVKLKNATYPDLSIKLLLQKILANLDESKLSYQPSEQPSMMVPRPYPAGNVLLRQEWVWNEISHWFQPGDIIITETGASAFGVNQTRFPVNTLGISQALWGSVGYTMGACLGAEFAVQEINKDKFPATKHRVILFMGDGAFQLTVQELSTIVKWGLTPYIFVMNNQGYSVDRFLHHRSDASYYDIQPWNYLGLLRVFGCTNYETKKIITVGEFRSMISDPNFATNDKIRMIEIMLPPRDVPQALLDRWVVEKEQSKQVQEENENSSAVNTPTPEFQPLLKKNQVGY

SEQ ID NO: 69 - protein, 563 aa, synthetic construct
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 70 - protein, 563 aa, synthetic construct
MTVSEFLLGRLNELSVNHLFGIPGDYVLPFFDELIDKNTGVKHVNSRNELNATYSADGYSRLNGFGAAAVTFGVGSLSTVNAISGAYADNSPLVVICGAPTREASKTVGTRLYHHLLGQDFDTSMKVMENITIKTLRLSSLETAAAEIDDLLITSFIHKKPVYLEIPFDLQGAELDIYPTSPLDFKQTVTNQKKLTAAVDQILKLIDKADSISSLVGPLLQRNNMILTTDKVISHINACVGTVFTGKISHFEDHPNAVGFYQGKVSEDYTIKMIDGADLNITFGVQHTEFDTGVFTDGIGVNQDSIHILNDMVIINGEYYFDVYLKDILPILAEKVTSLSPKQLEIDKSAKKFSFERRDKFTPTDNALTIDRMYVQFANFMDSGDVLVGDTGGYINSSQTEFKKDIKIHGCGNWGSLGAGFGMSVGASFAHSEVTSDKGQVISITGDGAFLMSAQELSTVIEHELDMTLIILDNSGYGAERQIHPGKERSYNDFLPWKYEQLAETFGDIDGVTTSSYVAQTENDLDGIFTELRGKKGVNVVRVKLDPWDSASFNVKFSEALQH

SEQ ID NO: 71 - protein, 559 aa, synthetic construct
MGARALRRERRLRWSPNWTRILPMQPQKTLTAGQALVRLLANYGVDTVFGIPGVHTLELYRGLPGSGIRHVLTRHEQGAGFMADGYARVSGKPGVCFVITGPGVTNVATAIGQAYADSVPLLVISSVNHSASLGKGWGCLHETQDQRAMTAPITAFSALALSPEQLPELIARAYAVFDSERPRPVHISIPLDVLAAPVAHDWSAAVARRPGRGVPCSEALRAAAERLAAARRPMLIAGGGALAAGEALAALSERLAAPLFTSVAGKGLLPPDAPLNAGASLCVAPGWEMIAEADLVLAVGTEMADTDFWRERLPLSGELIRVDIDPRKFNDFYPSAVALRGDARQTLEALLVRLPQEARDSAPAAARVARLRAEIRAAHAPLQALHQAILDRIAAALPADAFVSTDMTQLAYTGNYAFASRAPRSWLHPTGYGTLGYGLPAGIGAKLGAPQRPGLVLVGDGGFLYTAQELATASEELDSPLVVLLWNNDALGQIRDDMLGLDIEPVGVLPRNPDFALLGRAYGCAVRQPQDLDELERDLRAGFGQSGVTLIELRHACAR

SEQ ID NO: 72 - protein, 568 aa, synthetic construct
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAESGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 73 - protein, 551 aa, synthetic construct
MVQTVIEHVLSRLQALGINDIFGVAGDYAFPINDAVCASPNLRWVGNCNELNAAYAADGYARVKGFAALATTFAVGELSALNGIAGAYAERLPIFHLVGMPASYIQKNQPIMHHTLGDGDFEVFYKVAQFFSCVHTVLTPENCVSEMTRLIAQAFAQRRPVYIGIPADHAVKRIVVQSIKPFTLPSSRPDTLKKVLSLIVNKLSHSESPCALLGSLISRFDLVKEAEALIKITHLPYATMFMDKGVLNETHSQYIGMYSGHLMNPAIAEFIEGCDCILNIGAMMSDVNTGCFTADLDTNNMIHIMDNYVVIDAIRYSDVYIYELLAELRDILTFIPSSSIKVHGLGEPISNANGKITSDYLYPRFEQFIKQNDIVIADTGTFSMGLGFALLPKEVSFQTQSLWGSIGWATPAALGAALAAPHRRVILATGEGSHQLTAQEISLFARWGLKPLIFVLNNDGYLIERLFCHEPEAYYNDIHPWNYAQLPATLGCKNWYCQKITTCDELDRVLNTLERIDTAAYIEIVTDRYETSDLLQAMGKIIRAETPKHLK

SEQ ID NO: 74 - protein, 563 aa, synthetic construct
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVIRLIELKLPVFDAPESLIKQAQLTAATNAKQ

SEQ ID NO: 75 - protein, 547 aa, synthetic construct
MRRRIGQYLMDCLSNVGVDKVFGVPGDFNLTFLDDIIGRTDMEWIGNTNELNASYAADGYARMKGISAMVTTFGVGELSAVNGIAGAFAERVPVIQITGAPTQAVEQAGKFVHHSLGEGNFDDYRKMYEPITTTQAYITEANAQLEIPRVINAALTESRPVHIHLPIDVAAKEIEVMEDYHYKMSKPADVSQYIDMVQAKLNSAERPVIITGHEINSFGLHELLEQFVNQTGIPVAQLSLGKGAFNEESPYYMGIYDGSIAEERIKYYVDHSDAILNIGAKLTDSATAGFSYQFDIDDVIMLNHRNFKMNETRDDNVALQDLLIGLQSIDFHFNGDYPMFKKPVPNDDVLDDTPLTQQTYFKMMQDFLNRDDVVIAEQGTSFFGAYDLALTKTNKFIGQPLWGSIGYTLPATLGSQLADGERRNVLLIGDGSLQLTVQEMATMIRLQLKPVIFIINNDGYTVERKIHGENALYNDIPMWDYKALPSVFGGNETVSVHEVDSSVSLQHAFNEINLQPNRMHVIEVKMGMHDAPEKLNDIGKAFAKQNG

SEQ ID NO: 76 - protein, 387 aa, synthetic construct
MIDADELLGGLITRGVVDVVGVPCSYLTPMINRVASGSAAGYLPVTHEGEAVAIAAGCWLAGATACVIGQNSGLGNMVNPLTSLTHPSRIPVPLLVSWRGEPGFPDEPQHELMGEITPALLELMRVGHAILPAEAAELDKCLEAGWTEMDREQTPFAFILRNGVMAAQRLDEPPLADPVTATFIRSRRSRDAPTRMAALECLIGTLTDEAAVVSTTGKTSRELYTLADRPQHFYLVGAMGSASAVGLGVARHTSRPVVVIDGDGAALMRLGTLAAVGAHGGPNLTHVLLDNRVHDSTGGQRSLASQVDFPAVARACGYARVYDCADLDEFAEAVGAAQAEPGPAFVYLTIRPGSLAELGRPAVRPPEVARRFRDFVTGRGKDVDHVS

SEQ ID NO: 77 - protein, 547 aa, synthetic construct
MAQTVIEHVMWWLKQVGIVDVFGVPGDFSFPVTDAIIEDKDLKWVGCTNELNAAYAADGYARIRGMSALCTTYGVGELSAINGIAGAYSEYLPIVHLVGMPGMKTQAGKRVIHHSLGSGEFDFYYHMTKPVVCARTILTPENCVNEMERVMGCAVRERRPVYIAIPKDCAEMKIVGSGGDGEVEMQSDPDRLKAAVDDILARIRDVKGRGVFLPGMMAYRTGVMEVLQRLIEKTGFGFATMFHDKSVLDETHKQYVGMYDGQLMNQQVREYVEEADLLINVGALWGDFNTGMFTSNLDGHKTIDIYLDYAQVGFAVYEQVYMKDLISALADRVSGQDYVYELKAEGLGDAVGDPDEKISAEYLYPRWEQFLRKDDLLVCETGTVSMGMGFAKMPKGAVFCNQTLWCSIGWATPAAFGVAMGGSGRRVVLMTGEGAHQMTATEVGQFARYGVKPVIFVLNNDGYTIERLLCRRGEYEYNDVTVWKYDELPKAMGCEDWITARVTTNGELDTVLKKINENGHAAYVEIVVDQMAIPPLAKKIGDALGMK

SEQ ID NO: 78 - protein, 572 aa, synthetic construct
MKDNQQAVQLQQPTTIGHYLAVRLAQAGVKHHFVVPGDYNLGLLDKLQYNNYLEEVNCANELNCAFAAEGYARANGIAACVVTYSVGAFTAFDGIGGAYAEDLPVILISGSPNTNDIGSSHLLHHTLGTHDFSYQYEMAKKITCAAVSIQRPTEAPRLIDYAIKMALLKKKPVYIEVPTNVASQPCAAPGPASLITEPETSNQEYLQMAVDISAKIVNGKQKPVLLAGPKLRSFKAESAFLELANSLNCSVAVMPNAKSFFPESHPNYAGIYWGQASTLGAESIINWSDCIICAGTTFTDYSSNGWTSLPPKANVLHVDVDRVTVSDAEFGGVLLRDFLHELAKKVKANNASVVEYKRIRPESLEIPMENPKAALNRKEIIRQVQNLVNQETTLFVDTGDSWFGGMRITLPEKARFEIEMQWGHIGWSVPSAFGYAIGAPKRNVVVFVGDGSFQETVQEVSQMVRLNLPIIMFLINNRGYTIEVEIHDGPYNRIKNWDYAAIVEAFNAGEGHAKGFRVGNGHELAEAIRQAKENSQGPTLIECNIDQDDCSKELINWGHNVGAANGKPPAKE

SEQ ID NO: 79 - protein, 543 aa, synthetic construct
MQTTIGQYLLDRLKSYGVQHLFGVPGDYNLAFLDLVEDDPHLQWVGNCNELNASYAADGYARLKSMGALLTTFGVGELSAINGIAGSYAESVPVVKIVGMPSRGVVHSRKLVHHTLGDGEFLKFYNMYAEVSVAQTILNKQNAQSEIDRVLGECFLHKKPVYIGLPVDVTHIPIETYTSSPLVTRSDPKILNAFLEDAQELLRKSKSQVVMADYEVNRYHLNQELKHFIEAVNLPLVSLAMGKGVFDETHPNFIGVYNGILSDARVCSLMRHADCALLIGVKLTDSLTAGFHYIREHHLSIQIHPFYSQIGEKTYDDILMQDVLKALARLKFQASFPKETHPKTPHLNGKLTQDKFFKIIQEYLRPHDVLIAEQGTSFFGAISMVLPSGVSFIGQPLWGSIGYTFGALLGTALASPDRRHILLIGDGSFQLVAQELSTMLRENITPTIIVINNDGYTVERCIHGPTRQYNHINMWHYSKLASFFDVHLAREVVSFQVSSVAGLREALCVAQQSSKLALIEACMDKDDAPILLKKLGALFGAQI

SEQ ID NO: 80 - protein, 167 aa, synthetic construct
MNASEAVYKAILDSGVDFVTSVPCANLKTVLNYLNDDKDIQHIPVTREEEGIGVCTGAYLGGRKTALLMQNSGLGNSINAIGSLVKVYKIPILIIISHRGDLKEKISAQIPMGQWTKKLLETVEIPYFSPKTPDEAYKLIKDASELSINMEYPVAILLDALYWEHDK

SEQ ID NO: 81 - protein, 553 aa, synthetic construct
MTDNYKLSDYLLDVVKYMGSDDIFGVPGDYNLQLLDHITHRPDLSWKGNANELNASYMADGYAREKGFATFITTFGVGELSAINGLSGSIAEHVPVLEIVGAPTNKVQNNGSLVHHTFGDHNFDRFEKAHRALGIKSARLDSENAINQINEVAKYIYETKKPAYIILPTNLVEMEVNKSLQKDIPNLFIKDTIISDKSEKQIVSAINNSKNPVIIMGHEVDRFNVFDDIKSFASNNNIPVLDLGLGKGNIDESFDNFVGTYNGQISDDDINKYVENADTVILVGAKLTDSVTGGFTEQFGESNTIVLNYDNARIYGKDVDSDYNFKNIMNDLSTTKLEISLNAVNANNKEIELSPSDNTLTQSFYDNAIQNFASENNTIVAEQGTSFFGLADKRMPKGAKFFGQPLWGSIGYAFPASLGSQIANKDRRVVLSTGEGSLQLTIQELGLAFREKLNPVMFVIENSGYTVERVIHGMTEPYNDVPKLNYGLMPQAFGAKEDEYDFIEVSTEKELLDAMVTAKEETDKFVLIQVNMAVKDVPTPLAKTATAFANQNK

SEQ ID NO: 82 - protein, 546 aa, synthetic construct
MKRRVGQFLIDEIAKYNVDKIFGVPGDFNLAFLDDIIAHDQLEWVGNTNELNASYAADGYARIKGLSALVTTFGVGELSAINGIAGSYAENVPVIAITGAPTIQAEKSGRYIHHSLGTGTFDDYRKMFEPITTTQAYITTDNALEEIPRVIQAAYDEKRPVHIHLPIDVAMKEIDVPERVEYPEKTSFDLTATMSKVEKALKNAQQPTLIVGHQINSYHLHDEVQALAEKMNLPVAQLSFGKTAFKESSSQYMGIYDGSIAAPRIRDYVDQSDLIILLGAKLTDSATAGFSQGFDEASIITLNHKDIKIQNEVIQQPSLSDIVSAFKDIDYQFEGHYPKYEWPRENQYSLNDVPLTQKNYFDIMQSFIRSNDVILADQGTSFFGGYDLALPDGATFIGQPLWGSIGYTLPATLGAQLADRSRRHILLIGDGSLQLTVQEISTMIRQKLHPIIFVINNDGYTVERKIHGETEPYNDIQMWDYKALPALFGGRNVPTHDVKTSQELYDTLKQIENNPNQMHFVEVHMDVHDAPEKLEKISKVFAEQNK

SEQ ID NO: 83 - protein, 558 aa, synthetic construct
MTYTVGRYLADRLAQIGLKHHFAVAGDYNLVLLDQLLLNTDMQQIYCSNELNCGFSAEGYARANGAAAAIVTFSVGALSAFNALGGAYAENLPVILISGAPNANDHGTGHILHHTLGTTDYGYQLEMARHITCAAESIVAAEDAPAKIDHVIRTALREKKPAYLEIACNVAGAPCVRPGGIDALLSPPAPDEASLKAAVDAALAFIEQRGSVTMLVGSRIRAAGAQAQAVALADALGCAVTTMAAAKSFFPEDHPGYRGHYWGEVSSPGAQQAVEGADGVICLAPVFNDYATVGWSAWPKGDNVMLVERHAVTVGGVAYAGIDMRDFLTRLAAHTVRRDATARGGAYVTPQTPAAAPTAPLNNAEMARQIGALLTPRTTLTAETGDSWFNAVRMKLPHGARVELEMQWGHIGWSVPAAFGNALAAPERQHVLMVGDGSFQLTAQEVAQMIRHDLPVIIFLINNHGYTIEVMIHDGPYNNVKNWDYAGLMEVFNAGEGNGLGLRARTGGELAAAIEQARANRNGPTLIECTLDRDDCTQELVTWGKRVAAANARPPRAG

SEQ ID NO: 84 - protein, 547 aa, synthetic construct
MDTAHQIVKVLEDRDVKYVFGIPGEENIHLVDAINQSDKIKFILVRHEQGASFMAGVYGRLTGKPGVAAATLGPGAINLLLGVADSQTNSTPMIAITAQGGLDRIYKESHQVIDLKAMFQPITKWSTHIYEAKTTSEIIGKAYDKSVNGRPGPVYIGVPQDVEEQETGSQISTFSMPKESNEPNPKLIEKAARLIKNAKHPVVMAGMGIVRNHAEKELNAFIEKTHLPVATTFMGKGSVDDRSKYALGVIGFMAHDYGNFAFDQADLILSIGYELSEFNPVRINPKANKTIIHMSTFSEDSDEHYPVKVNIISDIDKSIRALSDCLNDFKSSETKADIREEVEAEIASGKVESKVPLTPQQIISATRESVDDKGIVLVDTGALKMWMARLYPTYAPNTCLIDNGLSTMSWSLPGAIGAKLAKPDHHILSVMGDGSFMMNSQELETAVRYHIPITILLWVDKSYGLIKWKMDMEMGHHSEVDFDNPDFVEYVESFGANAHIVSSRNDLKNKLQNYLKNDNQVNVLICPVDYRENMKLINKLGKVTVSL

SEQ ID NO: 85 - protein, 563 aa, synthetic construct
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 86 - protein, 604 aa, synthetic construct
MMEDSTNKICSQVLDSMEYTPGMSLDDMVTNTPCLGNMKCTDAFVRCLEAEGIKYIFGIPGEENLDFLESIRVAEKEGRIRLILTRHEQAAGFMAATVGRITGQPGVCLSTLGPGATNFSTAVAYAKLGGMPMLVLTGQKPIRHSKQGQFQIVDIVDHFRPITKSTKQVPDADAIPSLIRESVREALEEKPGPVHIELPEDIAEHILGPNEYLFPTGDVRRPVPDAKAIAKAVALIHAAKRPLLCIGSGCNRKRTSKMLRKIIDETGIFAVSTQMGKGVVDETSPHFLGALALSQNDWVHTAVDWADLIVNIGHDSVEKPPFLMKHNQKPIVIHVNFYPAIVDDVYFPHLEVVGDIGNAVWQISETLKSMPKPHWDFAVFEKIREVAQRRMHHSIYTTSDAFPMDIRRVVKDVRNCMPENSVVCLDNGMYKIWFARLYEAREPNTLLLDNALASMGAGLPSALAAKMVRPDHKVVAVSGDGGFMMNSQELETAVTLGLDVIQIVLNDNAYGMIEWKQSASGMPKWGLQLNNVDFVKYAEAYGAKGYRVTKADELTEILSKCLNEHGVHLIEVPVNYEASNRTLNVDVPDHVKELREILKKEFKY

SEQ ID NO: 87 - protein, 563 aa, synthetic construct
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAESGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 88 - protein, 547 aa, synthetic construct
MKKRVGEYLMDCLSNTGVEKVFGVPGDFNLAFLDDIISRDDIEWVGNTNELNASYAADGYARMKGISAMVTTFGVGELSAVNGVAGSYAERVPVIAITGAPTRAVENAGKYVHHSLGEGTFDNYRKMFKEITTAQGYITPDNAQTEIPRLLNAALAEKRPVHLHLPIDVAMTEIEVDTAYGVPERQPHDVSKYISMVKDKLESASQPVIIAGHEINSFKLHAALEKLVNQTHIPVAQLSLGKGAFNEENPYYMGIYDGSIAEENIRDYVDNSDAILNIGAKLTDSATAGFSFGFDIDDVVMLNQHNFKMNETVAEDVTLPDLMDGLLEMDYVNEADYPKFKRPEAGQYDLNGDVLTQETYFKMMQDFLAPSDVILAEQGTSFFGAYDLALYKDNKFVGQPLWGSIGYTLPATIGTQIADSNRRNLLLIGDGSLQLTVQGISTMIRQGLKPVMFVINNDGYTVERKIHGENEPYNDIYMWDYKALPAVFGGEDVVKVHDVSTSEELDQAFEAIKAYPDMMHFVEVKMAMHDAPHKLEAIGKAFAKQNS

SEQ ID NO: 89 - protein, 545 aa, synthetic construct
MNTAELLIRCLENEGVEYIFGLPGEENLHILEALKESPIRFITVRHEQGAAFMADVYGRLTGKAGVCLSTLGPGATNLMTGVADANLDGAPLIAITGQVGTDRMHIESHQYLDLVAMFAPVTKWNKQIVRPNTTPEVVRRAFKIAQQEKPGAVHIDLPENIAAMPVEGQPLQRDGREKIYASSRSLNRAAEAIAHAKSPLILVGNGIIRADAAEALTDFATQLNIPVVNTFMGKGAIPYTHPLSLWTVGLQQRDFVTCAFEQSDLVIAVGYDLIEYSPKRWNPEGTTPIIHIGEVAAEIDSSYIPLTEVVGDIGDALNEIRKRTDREGKTAPKFLNVRAEIREDYERHGTDASFPVKPQKIIYDLRQVMAPEDIVISDVGAHKMWMARHYHCDRPNTCLISNGFAAMGIAIPGAVAAKLVYPEKNVVAVTGDGGFMMNCQELETALRIGANFVTLIFNDGGYGLIGWKQINQFGAPAFVEFGNPDFVQFAESMGLKGYRITAAADLVPTLKEALAQDVPAVIDCPVDYSENVKFSQKSGDLICRM

SEQ ID NO: 90 - protein, 568 aa, synthetic construct
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 91 - protein, 548 aa, synthetic construct
MQKRVGQYLMDAISAVGVDKVFGVPGDFNLTFLDDIIHRDDMDWVGNTNELNASYAADGYARMKGISAMVTTFGVGELSAVNGIAGAYAERVPVIAITGAPTREVEKAGKYVHHSLGEGTFDDYRKMYKHITTAQGYITPENAQVEIPRLIDAALNEKRPVHMHLPIDVAAQKIEVEKPYCYVTPEKQDVSSYIKRIEDKLKTAKQPLIIAGHEINSFDLHEALEQFVNQTHIPVAQLSLGKGAFNEQNDYYMGIYDGEISDGPIKSYVNGSDVILNIGAKLTDSATAGFSYQFNLDDVIMINHHNFKVDALHAEEVRLTDLLEGLLEMDYVSDVNYPHYHVSEHKEIELIDHPLTQSTYFKLMQSFIKPKDIILAEQGTSFFGAYDLALYKENKFIGQPLWGSIGYTMPATLGTQIADKQRRNLLLIGDGSLQLTVQALSTMIREGLKPVIFVVNNDGYTVERKIHGETASYNDINMWDYKMLPFGFGMKEDDVKIHNVTTSIELEKTLKDIDDTPNIMHFVEVHMDIHDAPAKLNEIAKAFANQNN

SEQ ID NO: 92 - protein, 563 aa, synthetic construct
MSEITLGKYLFERLKQVNVNTVFGLPGGFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 93 - protein, 635 aa, synthetic construct
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 94 - protein, 558 aa, synthetic construct
MKTQYTVSTYLLDRLSELGIEHIFGVPGDYNLAFLDDVLAHEKLKWIGNCNELNAAYAADGYARIKGIAALITTFGVGELSALNGVAGSYAENLPVIKITGTPTTKLMENGALVHHTLGDGKFDHFSNMYREITVAQAKLTPEHAVEEIDRVLRACWIEKRPVHIQLPIDVYNKPINKPTEPILHKPVVSNKDALDKMLLHATSKISSAKKPVILADFEVARFHAEEYLHQFVEKTGFPIATLSMGKGVFPEKHPQFIGVYTGDVSSPYLRKRIDESDCIISIGVKLTDTITGGFTQGFTKEQVIEIHPYTVKIIDKTYGPVVMKDVLEQLTDLIEHRIEETLEIKSFISESLSITEEFNPKAQMVTQKRFWQQIYHFLQENDVLIAEQGTPFFGSATIPLPNNTTYVTQPLWGSIGYTLPALLGTQLADLSRRNILIIGDGSFQVTVQELSTILRQNLKPIIFLINNNGYTVERAIHGQKEPYNDIQMWDYTKLANVFGSEEKSLPCKVENEIELAEVLTNITLNKNQLIFIEVIMSQGDQPELLAKLGERFGKQNS

SEQ ID NO: 95 - protein, 577 aa, synthetic construct
MSSINSFTVADYLLTRLHQLGLRKVFQVPGDYVANFMDALEQFNGIEAVGDLTELGAGYAADGYARLTGIGAVSVQFGVGTFSVLNAIAGSYVERNPVVVITASPSTGNRKTIKETGVLFHHSTGDLLADSKVFANVTVAAEVLSDPSDARQKIDKALTLAITFRRPIYLEAWQDVWGLACEKPEGELKALPLISEEGALKAMLADSLKLLNSARQPLVLLGVEINRFGLQDAVLDLLKASGLPYSTTSLAKTVISENEGIFVGTYADGASFPATVEYIEKADCVLALGVIFTDDYLTMLSKQFDQMIVVNNDETSRLGHAYYHQLYLADFILQLTDEIKKSSLYPRQNSALPLLPPQPQITPALLQQQLSYQNFFDLFYGYLLQHQLQDNISLILGESSSLYMSARLYGLPQDSFIADAAWGSLGHETGCVTGIAYASDKRAMAIAGDGGFMMMCQCLSTISRHQLNSVVFVISNKVYAIEQSFVDICAFAKGGHFAPFDLLPTWDYLSLAKAFSVEGYRVQNGEELLQALEHIMTQKDKPALVEVVIQSQDLAPAMAGLVKSITGHTVEQCAIPT

SEQ ID NO: 96 - protein, 560 aa, synthetic construct
MSGTHRGRGADATIGGYLINRLQDYGIRHIFGIPGDYVLGFYSMLEESPIEVVGATREDCAGFAADAYARVGGMGALCVTYCVGGLSVCNSVAGAFAEKSPVVVISGAPGLNERRNNPLLHHKVRDFRTQMEVFERLCIAGTELRDPLTAFAEIERVLDACDRYKRPVYIEIPRDMVHVVPPAAQSFLRPREEIDQDALAEAVREAIERLNRAKKPVIIAGVEIHRFGLQDLVLELAEKAEIPIAATILGKSVIQETHPLYVGLYEGALGCDEVTDFVEESDCILLLGTFMTDINLGIYTAELDLRKCIYATSEQLQISHHFYRDIPLDAFLQALIAAEPHPSKRPIPEEIKPRIEPIEVEPARPLQISRMVKMLNSHLHKDNIVIADIGDALFASTELVVHERADFLSPAYYTSMGFSVPAIVGASCAKPDQRVIAICGDGAFQMTGQELSTVIRLGLSPILIILDNGGYVTEKFLHPGDWKYNDIHPWNYYKLPDIYGGGRGYKVQTEGEFAEALEQALEDQSCAQIIHAVMAPGDASHTLRCLAERLGQQVGGHPAS

SEQ ID NO: 97 - protein, 563 aa, synthetic construct
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLAHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAESGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 98 - protein, 591 aa, synthetic construct
MSDATPPTVSRYLLDRIAATGVTHVFGVPGDYNLTFLDRIEAHPQLRWVGNANELNAAYMADGYARLKGFAAVVTTYGVGELSAMNGIAGAYAENVPVLHIVGTPTCAIQRQGLPVHHSLLDGDHQHFVRAAREVTCAAASLTAADAAGEIDRVIAAILHEKRPGYLALPSDLVTRPCPARTEPDSAAAPSARTPLHPHAPAAAFGEAAARLLREATRITVLVDGLARRYDVGRQVDDLIRKGRLTAAVTTGGKGVIDESQDTFAGLYIGAISAPGTRAAVEDADVVIGVGLTVNDLNTGGFTARVDPARLIDLQPHHATVGATRIDRLPLHQAIDELGALLSALPDDPARPVVALPDDRARPITALPDDPARPVTAGVAGALAGTAAEYASAASATPLTQDVLWRRLGRFLRPGDIVAADQGTPFYGLLGQRLPADVEVIAQPGWSSIGYSLPAIAGAQLATAARRRAVLVIGDGAAQLTVQEFGTIAREGLDPIIIVVNNDGYTVERAINGPNAAYNDIARWEWQRIPAAFGADATCTRVRTVGELDTVLTELPRTTGRIRVVEAVLDRHDLPQVLRDTCVAVATRNAG

SEQ ID NO: 99 - protein, 528 aa, synthetic construct
MKTVHGATYDILRQHGLTTIFGNPGSNELPFLKGFPEDFRYILGLHEGAVVGMADGYALASGQPTFVNLHAAAGTGNGMGALTNAWYSHSPLVITAGQQVRSMIGVEAMLANVDAAQLPKPLVKWSHEPATAQDVPRALSQAIHTANLPPRGPVYVSIPYDDWACEAPSGVEHLARRQVSSAGLPSPAQLQHLCERLAAARNPVLVLGPDVDGSAANGLAVQLAEKLRMPAWVAPSASRCPFPTRHACFRGVLPAAIAGISHNLAGHDLILVVGAPVFRYHQFAPGNYLPAGCELLHLTCDPGEAARAPMGDALVGDIALTLEAVLDGVPQSVRQMPTALPAAEPVADDGGLLRPETVFDLLNALAPKDAIYVKESTSTVGAFWRRVEMREPGSYFFPAAGGLGFGLPAAVGVQLASPGRQVIGVIGDGSANYGITALWTAAQYNIPVVFIILKNGTYGALRWFADVLDVNDAPGLDVPGLDFCAIARGYGVQAVHAATGSAFAQALREALESDRPVLIEVPTQTIEP

SEQ ID NO: 100 - protein, 563 aa, synthetic construct
MSEITLGRYLFERLKQVEVQTIFGLPGSFNLSLLDNIYEVPGMRWAGNANELNAAYAADGYARLKGMSCIITTFGVGELSALNGIAGSYAEHVGVLHVVGVPSVSSQAKQLLLHHTLGNGDFTVFHRMSSNISETTAMITDINTAPAEIDRCIRTTYVSQRPVYLGLPANLVDLTVPASLLDTPIDLSLKPNDPEAEEEVIENVLQLIKEAKNPVILADACCSRHDAKAETKKLIDLTQFPAFVTPMGKGSIDEKHPRFGGVYVGTLSSPAVKEAVESADLVLSVGALLSDFNTGSFSYSYKTKNIVEFHSDYTKIRSATFPGVQMKFALQKLLTKVADAAKGYKPVPVPSEPEHNEAVADSTPLKQEWVWTQVGEFLREGDVVITETGTSAFGINQTHFPNNTYGISQVLWGSIGFTTGATLGAAFAAEEIDPKKRVILFIGDGSLQLTVQEISTMIRWGLKPYLFVLNNDGYTIERLIHGETAQYNCIQNWQHLELLPTFGAKDYEAVRVSTTGEWNKLTTDEKFQDNTRIRLIEVMLPTMDAPSNLVKQAQLTAATNAKN

SEQ ID NO: 101 - protein, 553 aa, synthetic construct
MKETVIEYILSKLYDLGIEDIFGVPGDYSFPINDAVCASSRQRWIGNCNELNAAYAADGYARIKGAAALSTTFGVGELSALNAIAGSYAERLPIFHLVGMPPSQSKKKRLINHHSLGDGDYDVFYKMSQHIVCAHAIITPENCIEETERLITCALHERRPVYFGLPADYAVKTVVKSPHNTVPLIPKTNPETLKNIIHLILNKLINSQQPCLLAGSISLRLGLQKSLQTIIEKTNIPYTTFYMDKSVLDETNTQYIGMYAGEFINPQVTSFVENCDCVLNIGTIMSDFNTACYTSNIEEGNIIHIMDNYVVISGQRYDEIYMKDVISSLKDKLISTSNYYFYHNNYPKAQSLGTPISSKTQEITETFLYPRLEKMLKPNDIIFGDTGTFTMGLSFALLPKGASFHSQSLWGSIGWATPAALGAALASPSSRLILITGEGAHQLTIQEISQFSRFGLKPIIFVLNNDGYLIERLFCKEPEYYYNDVTQWNYAQLPSAFGCDDWYCQKVATCDELNDALQHIETLDSAAYIEIMTDRYDASKYLVKMGEAITSTY

SEQ ID NO: 102 - protein, 547 aa, synthetic construct
MQKRVGQYLMDAIRSVGVDKVFGVPGDFNLTFLDDIINRDDMEWIGNTNELNASYAADGYARIKGISAMVTTFGVGELSAVNGIVGAYAERVPVIAITGAPTREVERAGKYVHHSLGEGTFDDYRKMYKPITTAQGYITPENAQTEIPRLINAAINEKRPVHIHLPIDVAAVNIEVQTPYQYTAPVKQDVDSYIDIIENKLKNAKQPLIITGHEINSFGLHDKLEQFVNQTHIPVAQLSLGKGAFNEENQYYMGIYDGKIADEQIKAYVDHSDVILNIGAKLTDSATAGFSYKFNLENVIMINHHHFKVDELRDEEVRLADLLDSLLGIDYTADVSYPRYQLTNETVELSDEPLKQSTYFKLMQQFIQPKDVILAEQGTSFFGAYDLLFSKDNTFIGQPLWGSIGYTLPATLGTQIADKQRRNVLLIGDGSLQLTVQALSTIIREDLKPVIFVINNDGYTVERKIHGESAPYNDIKMWDYKQLPFVFGMKDTEVKIHDVTTSNALATALNEINQTPNMMHFVEVHMDVHDAPEKLDAIGKAFAQQNG

SEQ ID NO: 103 - protein, 687 aa, synthetic construct [sequence omitted]

SEQ ID NO: 104 - protein, 387 aa, synthetic construct
MYVVNPEEKVIEIMKQTGIDLAATLPCDRIKNLLPLVSENFPEIKLTREENGVGICAGIYLAGGKPMMLIQSTGLGNMINALESLNVTCKIPLPILASWRGVYKEGIEAQVPLGAHLPSILEGAGLTYTIIGETEKLPLLENVILDAFENSRPHIALVSPKVWEASECCAWQAAGMPIKPEIMERTCRFSLTSGTLKPFMLRNDAICTLASELDDEITVTNLGVPCKELYACRDRELNFYMFGSMGLVSSIGLGLALRSEKTVITFDGDGSLLMNPNALLEIAKEAPKNLIIIALDNGAYGSTGSQETCALRYIDLEIFANACGIQNTAKVNSKEGVIEAFRKFKAMRELSFIHVILKPGNTNAPNIPMSPEEATKRFKETLDVKKF

SEQ ID NO: 105 - protein, 545 aa, synthetic construct
MKLAEALLRALKDRGAQAMFGIPGDFALPFFKVAEETQILPLHTLSHEPAVGFAADAAARYSSTLGVAAVTYGAGAFNMVNAVAGAYAEKSPVVVISGAPGTTEGNAGLLLHHQGRTLDTQFQVFKEITVAQARLDDPAKAPAEIARVLGAARALSRPVYLEIPRNMVNAEVEPVGDDPAWPVDRDALAACADEVLAAMRSATSPVLMVCVEVRRYGLEAKVAELAQRLGVPVVTTFMGRGLLADAPTPPLGTYIGVAGDAEITRLVEESDGLFLLGAILSDTNFAVSQRKIDLRKTIHAFDRAVTLGYHTYADIPLAGLVDALLEGLPPSDRTTRGKEPHAYPTGLQADGEPIAPMDIARAVNDRVRAGQEPLLIAADSGDCLFTAMDMIDAGLMAPGYYAGMGFGVPAGIGAQCVSGGKRILTVVGDGAFQMTGWELGNCRRLGIDPIVILFNNASWEMLRTFQPESAFNDLDDWRFADMAAGMGGDGVRVRTRAELKAALDKAFATRGRFQLIEAMIPRGVLSDTLARFVQGQKRLHAAPRE

SEQ ID NO: 106 - protein, 545 aa, synthetic construct
MKLAEALLRALKDRGAQAMFGIPGDFALPFFKVAEETQILPLHTLSHEPAVGFAADAAARYSSTLGVAAVTYGAGAFNMVNAVAGAYAEKSPVVVISGAPGTTEGNAGLLLHHQGRTLDTQFQVFKEITVAQARLDDPAKAPAEIARVLGAARALSRPVYLEIPRNMVNAEVEPVGDDPAWPVDRDALAACADEVLAAMRSATSPVLMVCVEVRRYGLEAKVAELAQRLGVPVVTTFMGRGLLADAPTPPLGTYIGVAGDAEITRLVEESDGLFLLGAILSDTNFAVSQRKIDLRKTIHAFDRAVTLGYHTYADIPLAGLVDALLEGLPPSDRTTRGKEPHAYPTGLQADGEPIAPMDIARAVNDRVRAGQEPLLIAADMGDCLFTAMDMIDAGLMAPGYYAGMGFGVPAGIGAQCVSGGKRILTVVGDGAFQMTGWELGNCRRLGIDPIVILFNNASWESLRTFQPESAFNDLDDWRFADMAAGMGGDGVRVRTRAELKAALDKAFATRGRFQLIEAMIPRGVLSDTLARFVQGQKRLHAAPRE

SEQ ID NO: 107 - protein, 397 aa, synthetic construct
MIGAADLVAGLTGLGVTTVAGVPCSYLTPLINRVISDPATRYLTVTQEGEAAAVAAGAWLGGGLGCAITQNSGLGNMTNPLTSLLHPARIPAVVITTWRGRPGEKDEPQHHLMGRITGDLLDLCDMEWSLIPDTTDELHTAFAACRASLAHRELPYGFLLPQGVVADEPLNETAPRSATGQVVRYARPGRSAARPTRIAALERLLAELPRDAAVVSTTGKSSRELYTLDDRDQHFYMVGAMGSAATVGLGVALHTPRPVVVVDGDGSVLMRLGSLATVGAHAPGNLVHLVLDNGVHDSTGGQRTLSSAVDLPAVAAACGYRAVHACTSLDDLSDALATALATDGPTLVHLAIRPGSLDGLGRPKVTPAEVARRFRAFVTTPPAGTATPVHAGGVTAR

SEQ ID NO: 108 - protein, 545 aa, synthetic construct
MKLAEALLRALKDRGAQAMFGIPGDFALPFFKVAEETQILPLHTLSHEPAVGFAADAAARYSSTLGVAAVTYGAGAFNMVNAVAGAYAEKSPVVVISGAPGTTEGNAGLLLHHQGRTLDTQFQVFKEITVAQARLDDPAKAPAEIARVLGAARALSRPVYLEIPRNMVNAEVEPVGDDPAWPVDRDALAACADEVLAAMRSATSPVLMVCVEVRRYGLEAKVAELAQRLGVPVVTTFMGRGLLADAPTPPLGTYIGVAGDAEITRLVEESDGLFLLGAILSDTNFAVSQRKIDLRKTIHAFDRAVTLGYHTYADIPLAGLVDALLEGLPPSDRTTRGKEPHAYPTGLQADGEPIAPMDIARAVNDRVRAGQEPLLIAADMGDCLFTAMDMIDAGLMAPGYYAGMGFGVPAGIGAQCVSGGKRILTVVGDGAFQMTGWELGNCRRLGIDPIVILFNNASWEMLRTFQPESAFNDLDDWRFADMAAGMGGDGVRVRTRAELKAALDKAFATRGRFQLIEAMIPRGVLSDTLARFVQGQKRLHAAPRE

SEQ ID NO: 109 - protein, 587 aa, synthetic construct
MTNILSKRALIASAPPVATDFTVADYLLTRLKQLNVTDVFQIPGDYVKQFTQALEHFDGVNTIGAINELDAAYAADAYARSRGLAAVSLQFGVSAYSALNAVAGAWVERSPIVVISATPGADMRDITDMYDVLFHHSTGDLDSDRKIYENVTVKAITLSTNVGAAEQIDELLVDAITNQRPVYIACYKEVWGQPCPRPSNTPLKPRVIHSPELALNNAVNQAWTKITLAKQPLILAGVEILRFGLSDLLQQIIDASGFLYTTTTLGKAVLDEQGDKFIGTYSDAASIQSVRDIVEASDCVLTLGAIITDDYLVLIETKYADMVLADTTGVRAGYFKYGDVTLHDFMKALLAKFKASKSGYPIKAKAPPQPQYPEPWASNSDPVYDAQPQVITYNRFFAQTMKFLQDENLLKEIVMTYGVSSAMYVASNCYGLSRGSFISSAAWQCIGFETGAAAGAQLGSGKRAWTVAGDGGFMMVCQSLSTLARNQLNAVIFVMSNEVYAIEQVYVDMTAFEPGPTHTFDTFDILPKWDYLALAKAFGAEGIRVETVDGLKAALPRIAGIKNKPVLVEVVIPQKDLPGQMYRLGSE

SEQ ID NO: 110 - protein, 563 aa, synthetic construct
MSEITLGRYLFERLKQVEVQTIFGLPGNFNLSLLDNIYEVPGMRWAGNANELNAAYAADGYARLKGMSCIITTFGVGELSALNGIAGSYAEHVGVLHVVGVPSVSSQAKQLLLHHTLGNGDFTVFHRMSSNISETTAMITDINTAPAEIDRCIRTTYVSQRPVYLGLPANLVDLTVPASLLDTPIDLSLKPNDPEAEEEVIENVLQLIKEAKNPVILADACCSRHDAKAETKKLIDLTQFPAFVTPMGKGSIDEKHPRFGGVYVGTLSSPAVKEAVESADLVLSVGALLSDFNTGSFSYSYKTKNIVEFHSDYTKIRSATFPGVQMKFALQKLLTKVADAAKGYKPVPVPSEPEHNEAVADSTPLKQEWVWTQVGEFLREGDVVITETGTSAFGINQTHFPNNTYGISQVLWGSIGFTTGATLGAAFAAEEIDPKKRVILFIGDGSLQLTVQEISTMIRWGLKPYLFVLNNDGYTIERLIHGETAQYNCIQNWQHLELLPTFGAKDYEAVRVSTTGEWNKLTTDEKFQDNTRIRLIEVMLPTMDAPSNLVKQAQLTAATNAKN

SEQ ID NO: 111 - protein, 188 aa, synthetic construct
MYPKRIDIIKKIVENVGEKEIIVSNIGIPSKELYYVKDRERNFYMLGSMGLASSIGLGLALNCEDKVIVIDGDGSILMNLGSLSTIGYMNPKNYILVIIDNSAYGSTGNQKTHTGKNTNLEEIAKGCGLDTITTESLEEFEKEFKNALNEEKCKVIIAKTIPYNEKCSNIEIPPVVLKYRFMEAIKRS

SEQ ID NO: 112 - protein, 563 aa, synthetic construct
MSEITLGKYLFERLKQVNVNTVFGLPGSFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 113 - protein, 545 aa, synthetic construct
MKLAEALLRALKDRGAQAMFGIPGDFALPFFKVAEETQILPLHTLSHEPAVGFAADAAARYSSTLGVAAVTYGAGAFNMVNAVAGAYAEKSPVVVISGAPGTTEGNAGLLLHHQGRTLDTQFQVFKEITVAQARLDDPAKAPAEIARVLGAARALSRPVYLEIPRNMVNAEVEPVGDDPAWPVDRDALAACADEVLAAMRSATSPVLMVCVEVRRYGLEAKVAELAQRLGVPVVTTFMGRGLLADAPTPPLGTYIGVAGDAEITRLVEESDGLFLLGAILSDTNFAVSQRKIDLRKTIHAFDRAVTLGYHTYADIPLAGLVDALLEGLPPSDRTTRGKEPHAYPTGLQADGEPIAPMDIARAVNDRVRAGQEPLLIAADMGDCLFTAMDMIDAGLMAPGYYAGMGFGVPAGIGAQCVSGGKRILTVVGDGAFQMTGWELGNCRRLGIDPIVILFNNASWEKLRTFQPESAFNDLDDWRFADMAAGMGGDGVRVRTRAELKAALDKAFATRGRFQLIEAMIPRGVLSDTLARFVQGQKRLHAAPRE

SEQ ID NO: 114 - protein, 563 aa, synthetic construct
MSEITLGKYLFERLKQVNVNTVFGLPGNFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 115 - protein, 551 aa, synthetic construct
MTDRRTIGSTLLDRLHRLGLRHIFGIPGDYVLSLFQLIEESPITHVGMTREDCAGFAADAYARIHGIGGICVTYCVGGLNTVNAVACAYAERSPVVVLSGSPGLSERVKNPLLHHMVRDFTTQKEVFEKVTVAAVVLDDPLTAEREIDRALAALMRYKRPVYLEIPRDLVHAPIESPAHPPAEQGEASDPAALAEAVAEVREMLASVKKPVLLVGAEVHRFHLQDEVSGLAERMNIPVASTLLGKSVIREDHPLFVGVYGGWVGHDEVQQFVNDSDCLVMLGSILTDLEELGADSPLMKAGRTVHATADAITVKHHRYEGVFFEDFVKALVAAPMPSFPARALPVHAIEPAHMPPPDQPATLKGLFGHLESLLDEKMLVIADIGESLFAGADLRVHRRTDFISPAYYTSMGFAVPAAVGAGFADPGLRPIVLVGDGAFQMTGSELSTCVRYSQAPIVIVLNNRGYSTEREILEGPFNDIHEWRYEKICDLIGGGVGYKVSTHGELVRALQAATSDKSQLHVLNVLLDPADRSPAMVRLAHRLAKRVAAGNR

SEQ ID NO: 116 - protein, 529 aa, synthetic construct
MTTVHAAAYELLRSNRLTTIFGNPGSNELPFLDAMPADFRYILGLHEGVVVGMADGFAQASGQAAFVNLHAASGTGNAMGALTNAWYSHTPLVITAGQQVRPMIGLEAMLSNVDAASLPRPLVKWSAEPAQAPDVPRALSQAIHTATSDPKGPVYLSIPYDDWNQDTGNLSEHLSSRSVSRAGNPSAEQLDDILSALREAANPALVFGPDVDAARANHHAVRLAEKLAAPVWIAPSAPRCPFPTRHPNFRGVLPASIAGISALLNGHDLIVVIGAPVFRYHQYQPGSYLPENSRLIHITCDAGEAARAPMGDALVADIGQTLRALADIIPQSKRPPLRPRVIPPVPDSQDDLLAPDAVFEVMNEVAPEDVVYVNESTSTVTALWERVELKHPGSYYFPASGGLGFGMPAAVGVQLANDRRRVIAVIGDGSANYGITALWTAAQEKIPVVFIILNNGTYGALRAFAKLLNAENAAGLDVPGICFCAIAEGYGVEAHRITSLENFKDKLSAALQSDTPTLLEVPTSTTSPF

SEQ ID NO: 117 - protein, 563 aa, synthetic construct
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAEGGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 118 - protein, 545 aa, synthetic construct
MKLAEALLRALKDRGAQAMFGIPGDFALPFFKVAEETQILPLHTLSHEPAVGFAADAAARYSSTLGVAAVTYGAGAFNMVNAVAGAYAEKSPVVVISGAPGTTEGNAGLLLHHQGRTLDTQFQVFKEITVAQARLDDPAKAPAEIARVLGAARALSRPVYLEIPRNMVNAEVEPVGDDPAWPVDRDALAACADEVLAAMRSATSPVLMVCVEVRRYGLEAKVAELAQRLGVPVVTTFMGRGLLADAPTPPLGTYIGVAGDAEITRLVEESDGLFLLGAILSDTNFAVSQRKIDLRKTIHAFDRAVTLGYHTYADIPLAGLVDALLEGLPPSDRTTRGKEPHAYPTGLQADGEPIAPMDIARAVNDRVRAGQEPLLIAADGGDCLFTAMDMIDAGLMAPGYYAGMGFGVPAGIGAQCVSGGKRILTVVGDGAFQMTGWELGNCRRLGIDPIVILFNNASWEMLRTFQPESAFNDLDDWRFADMAAGMGGDGVRVRTRAELKAALDKAFATRGRFQLIEAMIPRGVLSDTLARFVQGQKRLHAAPRE

SEQ ID NO: 119 - protein, 560 aa, synthetic construct
MTPQKSDACSDPVYTVGDYLLDRLAELGVSEIFGVPGDYNLQFLDHIVAHPTIRWVGSANELNAGYAADGYGRLRGMSAVVTTFGVGELSVTNAIAGSYAEHVPVVHIVGGPTKDAQGTRRALHHSLGDGDFEHFLRISREITCAQANLMPATAGREIDRVLSEVREQKRPGYILLSSDVARFPTEPPAAPLPRYPGGTSPRALSLFTKAAIELIADHQLTVLADLLVHRLQAVKELEALLAADVVPHATLMWGKSLLDESSPNFLGIYAGAASAERVRAAIEGAPVLVTAGVVFTDMVSGFFSQRIDPARTIDIGQYQSSVADQVFAPLEMSAALQALATILTGRGISSPPVVPPPAEPPPAMPARDEPLTQQMVWDRVCSALTPGNVVLADQGTSFYGMADHRLPQGVTFIGQPLWGSIGYTLPAAVGAAVAHPDRRTVLLIGDGAAQLTVQELGTFSREGLSPVIVVVNNDGYTVERAIHGETAPYNDIVSWNWTELPSALGVTNHLAFRAQTYGQLDDALTVAAARRDRMVLVEVVLPRLEIPRLLGQLVGSMAPQ

SEQ ID NO: 120 - protein, 563 aa, synthetic construct
MSEITLGRYLFERLKQVEVQTIFGLPGGFNLSLLDNIYEVPGMRWAGNANELNAAYAADGYARLKGMSCIITTFGVGELSALNGIAGSYAEHVGVLHVVGVPSVSSQAKQLLLHHTLGNGDFTVFHRMSSNISETTAMITDINTAPAEIDRCIRTTYVSQRPVYLGLPANLVDLTVPASLLDTPIDLSLKPNDPEAEEEVIENVLQLIKEAKNPVILADACCSRHDAKAETKKLIDLTQFPAFVTPMGKGSIDEKHPRFGGVYVGTLSSPAVKEAVESADLVLSVGALLSDFNTGSFSYSYKTKNIVEFHSDYTKIRSATFPGVQMKFALQKLLTKVADAAKGYKPVPVPSEPEHNEAVADSTPLKQEWVWTQVGEFLREGDVVITETGTSAFGINQTHFPNNTYGISQVLWGSIGFTTGATLGAAFAAEEIDPKKRVILFIGDGSLQLTVQEISTMIRWGLKPYLFVLNNDGYTIERLIHGETAQYNCIQNWQHLELLPTFGAKDYEAVRVSTTGEWNKLTTDEKFQDNTRIRLIEVMLPTMDAPSNLVKQAQLTAATNAKN

SEQ ID NO: 121 - protein, 568 aa, synthetic construct
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLNHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 122 - protein, 933 aa, synthetic construct
MQNSALKAWLDSSYLSGANQSWIEQLYEDFLTDPDSVDANWRSTFQQLPGTGVKPDQFHSQTREYFRRLAKDASRYSSTISDPDTNVKQVKVLQLINAYRFRGHQHANLDPLGLWQQDKVADLDPSFHDLTEADFQETFNVGSFASGKETMKLGELLEALKQTYCGPIGAEYMHITSTEEKRWIQQRIESGRATFNSEEKKRFLSELTAAEGLERYLGAKFPGAKRFSLEGGDALIPMLKEMIRHAGNSGTREVVLGMAHRGRLNVLVNVLGKKPQDLFDEFAGKHKEHLGTGDVKYHMGFSSDFQTDGGLVHLALAFNPSHLEIVSPVVIGSVRARLDRLDEPSSNKVLPITIHGDAAVTGQGVVQETLNMSKARGYEVGGTVRIVINNQVGFTTSNPLDARSTPYCTDIGKMVQAPIFHVNADDPEAVAFVTRLALDFRNTFKRDVFIDLVCYRRHGHNEADEPSATQPLMYQKIKKHPTPRKIYADKLEQEKVATLEDATEMVNLYRDALDAGDCVVAEWRPMNMHSFTWSPYLNHEWDEEYPNKVEMKRLQELAKRISTVPEAVEMQSRVAKIYGDRQAMAAGEKLFDWGGAENLAYATLVDEGIPVRLSGEDSGRGTFFHRHAVIHNQSNGSTYTPLQHIHNGQGAFRVWDSVLSEEAVLAFEYGYATAEPRTLTIWEAQFGDFANGAQVVIDQFISSGEQKWGRMCGLVMLLPHGYEGQGPEHSSARLERYLQLCAEQNMQVCVPSTPAQVYHMLRRQALRGMRRPLVVMSPKSLLRHPLAVSSLEELANGTFLPAIGEIDELDPKGVKRVVMCSGKVYYDLLEQRRKNNQHDVAIVRIEQLYPFPHKAMQEVLQQFAHVKDFVWCQEEPLNQGAWYCSQHHFREVIPFGASLRYAGRPASASPAVGYMSVHQKQQQDLVNDALNVE

SEQ ID NO: 123 - protein, 528 aa, synthetic construct [sequence omitted]

SEQ ID NO: 124 - protein, 220 aa, synthetic construct
MSRPLLQLALDHSSLEAAQRDVTLLKDSVDIVEAGTILCLNEGLGAVKALREQCPDKIIVADWKVADAGETLAQQAFGAGANWMTIICAAPLATVEKGHAMAQRCGGEIQIELFGNWTLDDARDWHRIGVRQAIYHRGRDAQASGQQWGEADLARMKALSDIGLELSITGGITPADLPLFKDIRVKAFIAGRALAGAANPAQVAGDFHAQIDAIWGGARA

SEQ ID NO: 125 - protein, 563 aa, synthetic construct
MSEITLGRYLFERLKQVEVQTIFGLPGNFNLSLLDNIYEVPGMRWAGNANELNAAYAADGYARLKGMSCIITTFGVGELSALNGIAGSYAEHVGVLHVVGVPSVSSQAKQLLLNHTLGNGDFTVFHRMSSNISETTAMITDINTAPAEIDRCIRTTYVSQRPVYLGLPANLVDLTVPASLLDTPIDLSLKPNDPEAEEEVIENVLQLIKEAKNPVILADACCSRHDAKAETKKLIDLTQFPAFVTPMGKGSIDEKHPRFGGVYVGTLSSPAVKEAVESADLVLSVGALLSDFNTGSFSYSYKTKNIVEFHSDYTKIRSATFPGVQMKFALQKLLTKVADAAKGYKPVPVPSEPEHNEAVADSTPLKQEWVWTQVGEFLREGDVVITETGTSAFGINQTHFPNNTYGISQVLWGSIGFTTGATLGAAFAAEEIDPKKRVILFIGDGSLQLTVQEISTMIRWGLKPYLFVLNNDGYTIERLIHGETAQYNCIQNWQHLELLPTFGAKDYEAVRVSTTGEWNKLTTDEKFQDNTRIRLIEVMLPTMDAPSNLVKQAQLTAATNAKN

SEQ ID NO: 126 - protein, 400 aa, synthetic construct
MISAADMVAGLTGLGVTTVAGVPCSYLTPLINRFISDPASRYLTVTQEGEAAAVAAGAWLGGGLGCAIAQNSGLGNMTNPLTSLLHPARIPAVVITTWRGRPGEKDEPQHHLMGRVTGDLLDLCDMEWALVPDTADGLHTALSACRDSMAYRELPYAFLLPQGVVADEPLDEAAPGSEPGQVIRHARIRPPGAPPTRIAALERLLSELPRAAAVVSTTGKTSRELYTLDDRDQHFYMVGAMGSAVTVGLGVALHTPRPVVVVDGDGSVLMRLGSLATVGAHAPRNLVHLVLDNGVHDSTGGQRTLSSAVDLPAVAAACGYRAVHACASLDDLSAALSAALAADGPTLVHLPIRPGSLAGLGRPKVTPAEVARRFRAFVTESPSMPPVGAGVGAPGRGAGR

SEQ ID NO: 127 - protein, 551 aa, synthetic construct
MYTVADYIADRLAELHIKDVFGVPGDFNLEFLDHITGHDALHWVGNANELNAGYAADGYARMNGIGAVVTTFGVGELSAINAIAGSFSENVPVVHIVGAPSKDAQASRRLLHHSLGDGDFSHFRRMASEVTCAVADLTPADAVDEVDRVLRAVQTHRRPGYIVVPTDVARVEVEPPSSPLGGARSYTSDRALAEFRAAASEFLEGRDVTVLADLLVHRMGATENLNRLLSGGIPHATLMWGKTLVNEEDPFFLGVYAGAASEPDVRKAVEGADRLVMAGVRFTDTTTASFSQKIDPARCVDIATNYAKVGDKTFAPLNIADALDALAELAPSFADRAPGAVPEPEPHHWVADADGPLVQDDVWPTLAGVLTEGNIVVADQGTSFFGLAKMRFPADTTFIGQPLWGSIGYTLPAAMGAGLACPGRRPVLVIGDGSAQLTVQELGTWMREGVNGVVIVVNNDGYAVERSIHGEDAEYNDITAWDWRVIPQALGGTDDKVVTLKATTRTELNDAFEKAQDNQDRLVLIEVCTAPKDYPKLLKKIGKAINDANSK

SEQ ID NO: 128 - protein, 429 aa, synthetic construct
MKGTIFAVALNHRSQLDAWQEAFQQSPYKAPPKTAVWFIKPRNTVIGCGEPIPFPQGEKVLSGATVALIVGKTATKVREEDAAEYIAGYALANDVSLPEESFYRPAIKAKCRDGFCPIGETVALSNVDNLTIYTEINGRPADHWNTADLQRNAAQLLSALSEFATLNPGDAILLGTPQARVEIQPGDRVRVLAEGFPPLENPVVDEREVTTRKSFPTLPHPHGTLFALGLNYADHASELEFKPPEEPLVFLKAPNTLTGDNQTSVRPNNIEYMHYEAELVVVIGKQARNVSEADAMDYVAGYTVCNDYAIRDYLENYYRPNLRVKSRDGLTPMLSTIVPKEAIPDPHNLTLRTFVNGELRQQGTTADLIFSVPFLIAYLSEFMTLNPGDMIATGTPKGLSDVVPGDEVVVEVEGVGRLVNRIVSEETAK

SEQ ID NO: 129 - protein, 401 aa, synthetic construct
MISASDMLAGLTGLGVTTVAGVPCSYLTPLINRVISDRATRYLTVTQEGEAAAVAAGSWLGGGLGCAITQNSGLGNMTNPLTSLLHPARIPAVVISTWRGRPGEKDEPQHHLMGRVTGDLFGLCDMEWSLLPDTPDALRGEFDVCREALARRELPYGFLLPQGVIADEPLDEEAPRSRAGRLVRHARTGPSDAAPTRVAALERLLAELPPAAAVVSTTGKTSRELYTLDDRDQHFYMVGAMGSAATVGLGVALHTPRPVVVVDGDGSALMRLGSLATVAAHAPGNLVHLILDNGVHDSTGGQRTLSSAVDLPAVAAACGYRAVHACGSLDDLTTALAGALATDGPTLIHLPIRPGSLAALGRPKVQPHEVARRFREFATEPWPASAVGSGTRAAAGSAGDR

SEQ ID NO: 130 - protein, 580 aa, synthetic construct
MTVNPITHYIGSFIDEFALSGITDAVVCPGSRSTPLAVLCAAHPDISVHVQIDERSAGFFALGLAKAKQRPVLLICTSGTAAANFYPAVVEAHYSRVPIIVLTADRPHELREVGAPQAINQHFLFGNFVKFFTDSALPEESPQMLRYIRTLASRAAGEAQKRPMGPVHVNVPLREPLMPDLSDEPFGRMRTGRHVSVKTGTQSVDRESLSDVAEMLAEAEKGMIVCGELHSDADKENIIALSKALQYPILADPLSNLRNGVHDKSTVIDAYDSFLKDDELKRKLRPDVVIRFGPMPVSKPVFLWLKDDPTIQQIVIDEDGGWRDPTQASAHMIHCNASVFAEEIMAGLTAATRSSEWLEKWQFVNGRFREHLQTISSEDVSFEGNLYRILQHLVPENSSLFVGNSMPIRDVDTFFEKQDRPFRIYSNRGANGIDGVVSSAMGVCEGTKAPVTLVIGDLSFYHDLNGLLAAKKLGIPLTVILVNNDGGGIFSFLPQASEKTHFEDLFGTPTGLDFKHAAALYGGTYSCPASWDEFKTAYAPQADKPGLHLIEIKTDRQSRVQLHRDMLNEAVREVKKQWEL

SEQ ID NO: 131 - protein, 1224 aa, synthetic construct
MNISFKNMIIGVANRGIPALRVGRTIREMGAIYAAFFTPVDKTAPHVSKADRAYSLSSVDGYLDIKEIVRVAKKHNVAALHPGWGFAAEDNNFPSMCEKHDIIFIGPSEGPMRKLGNKVKARDIARSLSIPVVEGSDVAVSLEEAKKIAEEIGFPVMIKSEGGGGGRGVVVVNSQQELDRHFYRASSMAEASFGNPNLYIEKYLSLVRHLEIQVICDKFGNVVALDERDCSCQRKYQKLLEITPSPWKLASLELRNALKEAAIKMVTAVEYDSIGTIEFLVDDKQNFYFIEANTRLQVEHGISELLYGIDIVEEMIRIAFGEKLSLTKEQLKSRGFAMQCRINFEDPQHDFMPNSGTITRYLSPGGEGVRLDSCVFGDYEFPKAYDSLGALLMTYGSTWEKTVSTMERALSEYFIGGLKTTIPFHKKIVTHPEFRSGEVHTKFIEGTPELISYREIIPEHLRLAGLMAEIAAMGYNPHIGLGRYRKPDDSRVGPMPVTQVSSSEKPTNNDYRPPFNPDDHRDMVLAYLRESEFVEFCNTTPRDVTQSESGNRFRLFDDRLIGPLIDQCGYVSIENGGGAHYHVAMLGCMTDPWAEAEEWNRFAPNTQKLILIRSTNVLGYAPQPREVMERTGQMIVKHYHVIRCFDFLNHIDNMAPFAKIVLKSENRIFQPAISLSWAEGFTIEHYLSVLNDILSMAGRILECGKAEASKKIICCLKDMAGVCPPRFIYDLISAILDNYPELVIQYHRHTTDGLAVSALGSAAKAGAKILDVADGPAVRFYSQAAVMPLVAYIEGELGLKTRLDKDKIRETAFVLKQIMPIYDRYCRPTFLGPDHDVTRHGLPGGATSSSQEGALKQGYAFLLPHILLILELYRKIIRYHDVTPGSQITWTNGYMMAVKAYERGGLAEVQRVINILKAVTSTPEQELDERIKEDRQILFAHANDALKNLLLGKFGKLPLGWPSDWVYQSVFGDKWRDAMAKRTEQSPLTVLPPVDMKKVEAELAGHIGRTPTENEIVNYLNHPGDALKLIKNLEKYGDPNVLPDDIWFEGLQTGYDREFVTSDGKVHTIKILRVGKVDRYGRKRVRYKLDYEIFVEDIKVKEKAVTGPGREMADTQNPYHIAAPFDSDLWLVHKKEGDAVKAGEEVLNLSLMKTEYAVTSPVDGVVKRIVVFADYRADKKMVPVKKGALLMELAPPRERCKNCDAEIEEDYKFCPKCGSNLESQ

SEQ ID NO: 132 - protein, 963 aa, synthetic construct
MAARNLEKMASIDAQLRLLAPGKVSEDDKLIEYDALLLDRFLDILQDLHGEDVREFVQECYEVAADYDGNRNTEKLEELGNMLTSLDPGDSIVVTKSFSNMLSLANLAEEVQIAYRRRIKKLKKGDFADEASATTESDIEETLKRLLQLNKTPEEVFDALKNQTVDLVLTAHPTQSVRRSLLQKFGRIRDCLTQLYAKDITPDDKQELDEALQREIQAAFRTDEIRRTPPTPQDEMRAGMSYFHETIWKGVPKFLRRVDTALKNIGINERVPYNAPLIQFSSWMGGDRDGNPRVTPEVTRDVCLLARMMAANLYFSQIEDLMFEMSMWRCNEELRVRAERQRCAKRDAKHYIEFWKQIPANEPYRAILGDVRDKLYNTRERARQLLSSGVSDVPEDAVFTSVDQFLEPLELCYRSLCDCGDRPIADGSLLDFLRQVSTFGLALVKLDIRQESERHSDVLDAITTHLGIGSYKEWSEDKRQEWLLSELSGKRPLFGPDLPKTEEVADVLDTFKVISELPSDSFGAYIISMATAPSDVLAVELLQRECGITDPLRVVPLFEKLADLESAPAAVARLFSIEWYRNRINGKQEVMIGYSDSGKDAGRLSAAWQLYKTQEELVKVAKEYGVKLTMFHGRGGTVGRGGGPTHLAILSQPPDTIHGQLRVTVQGEVIEQSFGEEHLCFRTLQRFTAATLEHGMHPPVSPKPEWRVLMDEMAIIATEEYRSVVFKEPRFVEYFRLATPELEYGRMNIGSRPSKRKPSGGIESLRAIPWIFAWTQTRFHLPVWLGFGGAFKRVIQKDSKNLNMLKEMYNQWPFFRVTIDLVEMVFAKGDPGIAALYDRLLVSEELQPFGEQLRVNYQETRRLLLQVAGHKDILEGDPYLRQRLQLRDPYITTLNVCQAYTLKQIRDPSFHVKVRPHLSKDYMESSPAAELVKLNPKSEYAPGLEDTVILTMKGIAAGMQNTG

SEQ ID NO: 133 - protein, 311 aa, synthetic construct
MKVAVLGAAGGIGQALALLLKLQLPAGTDLSLYDIAPVTPGVAVDVSHIPTAVNVKGFSGEDPTPALEGADVVLISAGVARKPGMDRSDLFNINAGIVRGLIEKVAVTCPKACVGIITNPVNTTVAIAAEVLKKAGVYDKRKLFGVTTLDVLRSETFVAELKGLNVSRTSVPVIGGHSGVTILPLLSQVQYAKWNEDEIEPLTKRIQNAGTEVLNAKAGGGSATLSMAQAAARFARSLVKGLSGETVVECTYVEGDGKYARFFSQPVRLGKEGVEEILPIGPLSNFEQQALENMLPTLRADIELGEKFING

SEQ ID NO: 134 - protein, 315 aa, synthetic construct
MKVSLIGAGNIGGTLAYLIASKKLASEVELIDVNGDLARGKALDVSQTLPLIGYTMKINGSANMERIKGSSVIIITAGIPRKPGMTREELIDVNAVVMKEVGEKIKKFAPKAFVIVVTNPLDVMVWVLYKAAEISPDKIVGMAGVLDASRMNLFLAQELGVSVADVKSLVLGSHGDSMVPLFRHSTVSGMSLPELVSVGLITKDKVDSIIERTRSGGAEIVALLKTGSAYYTPAASVLEMAEAYLLDQKKTLVCSVMMRGRYGVEDDIFSGIPVIMGSGGVERVIELDLTPDERRMFENSVAATRKLVLEARKYF

SEQ ID NO: 135-180 - skipped (46 entries)

SEQ ID NO: 181 - DNA, 1000 bp, synthetic construct
  source 1..1000: mol_type = other DNA
gagatgtaaaagaaagaaaatgaatttaatgaataagagaattgcaaatatttaaatagtccacccaagcatcatctaccttctaacattatcgaaaaaccaaaaaatcatccatcgcgccaactaacgccagattgtacatgagtgttagggtgaacttttaaaaccagtgcgcttttaataactcgtccctacgcatttcaatcattttaggagacccttctccgccttcactcatacctcattagctgcacgagttttcatgcgcacatatacatttgagtcctctctacctccgacttgaatgatttttaaagaaacgttgaaataagggtcaacagatttaatcgaggagatctgctcccgaagttcttctcgaactttgctaattcaactgttagacaaaactatatatacacgataaaaaagacataccgggccaaagaagtaccctccaaaagcaaagccatcaaaactattaccgttgtggtgacggacaaagaacaaacaaaagcagctaaggtgggtatatcagcgaaaatctcagtccacaatctcaatccgatctgataacggccatcaaaaatggttcaatgatacaactaaatatttttcatcatttaataaaccagatatatatttttaaattaacgaaaaacgatccatttcatatatacagcgttgatcgtttccgcccaataaatcaaaatcgccaagaacgacgttcgctaagctaatgtttattgcggtggcaaattcggctaagtgtactctaaagacaaaaggttaagaagcaaagaaaaattttaaatgacatcatgactttcctgcgcaaacaaaattcgttcgctgataggctcacgacaatattttaagccggaaaaaaagaaaaaaactttcgagacgtaacgaacgttatgtgactgctttttttgcgcaagtatatatgtaggcgctggcgcccttcgcttctacctagagtctgtttcatcgaactcac

SEQ ID NO: 182 - DNA, 982 bp, synthetic construct
  source 1..982: mol_type = other DNA
tggtacgtttgatgtgccgtttgtcgagccgtcgactgctagtaatattgtcattgtgattgccattgccattgtcaatgtcactgctattgctactgacactgccgctgtactctccttccgatgtagctttatttacattgtatcggaaaccgtccacacccatggtcgagagctgtgggtgacgcagacattcgaatggcatgccctacaacaactaagaaaatggctatcatgcggaagctggtgagaagaacagcatcgggacaagggaaggaagaacaaagacaaagaaaacaaaagaaagcaattgaaaacaaaataaaacaaaacaattttcattccttctcttctcttctcttcccattccttttcttttcttttcttttctctcattcaacgcactccatcgtatccttattcctcttattttttctctttctctatatcccatttctttctctctaggtgtgtcctctctctctctcttcaatttctctactctgcattccaacgcatccttcccccaaactcccatttcctccttacggcccgatagcgatcgcctttccctcgctatcactcgctaccggcccctcctctgcaccgtaacctcctacgtatttaccatatcataaagttttttccgacgcttatcgctgaccccctgtcgccctcctattggcttccggattatcttcttgtccataaggtgatccatgcttcctgaagattcccgaaatgtgtccactttggcggggaatcattccatccacttctttctctctcgctttcctcattcggcgctccccttccgcgtctcattggtcttccgctccgtttttgctttgccgatgttacttggggagaggtgcgataatcctttcgcaaaaactcggtttgacgcctcccatggtataaatagtgggtggtggacaggtgccctcgcttttctctaagcaagagaattccattgtcttga

SEQ ID NO: 183 - DNA, 1000 bp, synthetic construct
  source 1..1000: mol_type = other DNA
actatgctcctgacactttcctttttctacccatcctttctaccttttgtgcctctgtcctctgcctctgccccctgtcctctcccctagcgcttctacccactagccagacatatattcccagccctgtctctttctcatctagcataacatactactttgcagtacgccgttttgcaaggtaattacgctctactctgcagtagttccttactgacctcttaaagaaagcgctgctaagtttcgcaaaagccgatgattggcgggcaactccctatctcggttgctacataatcgtccagaatggcaagatgttgcttaaaactatgtcaggttacgtcggttcctggtaacgagatattgatagcaagtcacattgcagaacgagttcgaagcttttggaaattcctcagccgacggaagcagcgagagttaatgtaatgcgttattcacgcagaggtgccaaatgtaaacaaacaccgcaagttggatgtttaggtggatttttcgcgtctttctcatgcttatacgggtagtagtagagataggagagtttgggacgcgtcaatgtgtgttgttgcgcacggtatggaattagggaaacaactggggcgacgggatgtggaagggattcacgtcgctccaccggctgggtgacggtggagaatggaacggtaagtgagtcaggtgcatataacttaaaaaaatacagcgatttgatacttagagttgtaagaaaaagcggtatgtttttagaattccgggtacgtgtaggctttttccgaaaaacgaccgtatggttacgatattgttccgaagttacatttcccggcctttatatacaggcaatcccccgaccaaaaaggtacctctaccaaccactaaacccacaacatcaaagtagcctgcagaaaaaaattgaaattgttcttttttttctaaagctctttatataatactctcctacatttattttatcatataatataggttttgaaag

SEQ ID NO: 184 - DNA, 1000 bp, synthetic construct; identical to SEQ ID NO: 183
  source 1..1000: mol_type = other DNA

SEQ ID NO: 185 - DNA, 250 bp, synthetic construct
  source 1..250: mol_type = other DNA
gggaatgagaatgtgatccacttttaattcctaatgaatacatgcctatagttcttttcttttgttctttatgtcgtttttcgatggtacggccgttgtcaatctcagtttgtgtgcttggttgcagcttggtttcaaatctgttcatctcatgaatcttttaccatttcaccacacgtttataccattctctcatagaatcttcatcaaaccatctcggggttagagtggaaagaaagtcttgttcttt

SEQ ID NO: 186 - DNA, 1000 bp, synthetic construct
  source 1..1000: mol_type = other DNA
attaaaacggatgttgtttggtaagaatatgttacatttagattacaagaattatttggatattagaaatacatgaatgattatcctagataatataaatcataattataaaggatattttgaaaaacttgtctggtctcgtaaattttttaatagttttttagtagagtgatgtgttataataattagcagagtttgcgtagacttcgatatatatatatatatatattttttttttgtttacattctagtgcgatacaagagatagcgaaggtatcttagcttgctagaaaagtggattatgcaagttgtttgaaagttttagcaattagatatttacaagaaggactgaaatgattacaattttctttattatcgtggctgaatttctttgtcaaaataattaaaagaaaagaatgactattaattaaaatagaagaagtctgcctctttgccttattttgaaggctgtttaaacgagtgtttgaattagtaaatgacgactaagtataaccgttgactttttaataagccagataaagaatgtgttagaaagcgggttctggcataaaagtcaaaaagctttcaatccattgtaataataaataattaataaattaaaaattgagtgccgttcgagcttaaggaaacgtagtaatttaaacatttgcagtaccttcatccaatccaccattaccttttccaaaaggtaagaaagatttgtcacaagtggaagtgtaggagtatgaagttgtacgaacgggaccagttgaagaagaattaaaggcaaagtcaaatccaccaatgtaagctccattagccaaaacaaatctaacggggacaaagctatcctgttcaagataacctaaatcaaatgttcccataccataagcacctattctccagtgagcgtatgcatcatagttagtgttagaccatccagaaattgccttgtcaccgaaccaaccgaagaaagcatcgtcgacgttcttgacgaaca